(19) United States
(12) Patent Application Publication
(10) Pub. No.: US 2026/0176339 A1
(43) Pub. Date: Jun. 25, 2026

(54) FC-Containing Compositions And Methods For Increasing Transport Into The Brain

(71) Applicant: CEPHALON LLC, Frazer, PA (US)

(72) Inventors: Julia ROZENFELD, Frazer, PA (US); llze Anna Simpson, Frazer, PA (US)

(21) Appl. No.: 19/197,477

(22) Filed: May 2, 2025

Related U.S. Application Data
(60) Provisional application No. 63/642,308, filed on May 3, 2024.

(51) Int. Cl. C07K 16/18; A61K 39/00; C12N 9/24

(52) U.S. Cl. CPC C07K 16/18; C12N 9/2402; C12Y 302/01031; A61K 2039/505; C07K 2317/52; C07K 2317/77; C07K 2317/92; C07K 2319/00

(57) ABSTRACT

The present disclosure generally relates to compositions and methods for enhancing transport of molecules into the brain using modified IgG Fc regions, and methods of producing and using molecules comprising such modified Fc regions.

### CROSS-REFERENCE

This application claims the priority benefit of U.S. Provisional Patent Application No. 63/642,308, filed May 3, 2024, all of which are hereby incorporated herein by reference in their entirety.

### SEQUENCE LISTING

The instant application contains a Sequence Listing which has been submitted electronically in ASCII format and is hereby incorporated by reference in its entirety. The contents of the electronic sequence listing 035680-505001US 2026-03-06_SL_ST26.xml; Size: 439,396 bytes; and Date of Creation: Mar. 6, 2026.

### FIELD

The present disclosure generally relates to compositions and methods for enhancing transport of molecules into the brain using modified IgG Fc regions, and methods of producing and using molecules comprising such modified Fc regions.

### BACKGROUND

Treatment modalities for brain and neurological diseases are extremely limited due to the impermeability of the brain's blood vessels to most substances carried in the blood stream. The blood vessels of the brain, referred to collectively as the blood-brain barrier (BBB), are unique when compared to the blood vessels found in the periphery of the body. Tight apposition of BBB endothelial cells (EC) to neural cells like astrocytes, pericytes and neurons induces phenotypic features that contribute to the observed impermeability. Tight junctions between ECs comprising the BBB limit paracellular transport, while the lack of pinocytotic vesicles and fenestrae limit non-specific transcellular transport. These factors combine to restrict molecular flux from the blood to the brain to those molecules that are less than 500 Daltons and also lipophilic. Thus, using the large mass transfer surface area (over 21 m² from 400 miles of capillaries in human brain) of the bloodstream as a delivery vehicle is largely infeasible except in those circumstances where a drug with the desired pharmacological properties fortuitously possesses the size and lipophilicity attributes allowing it to pass freely through the blood vessel. Because of such restrictions, it has been estimated that greater than 98% of all small molecule pharmaceuticals and nearly 100% of the emerging class of protein and gene therapeutics do not cross the BBB in substantial amounts.

There is therefore a need for molecules with enhanced transport into the brain to deliver a therapeutic agent to the brain. The present disclosure provides molecules comprising modified Fc regions that solve the problems and meet the needs in the field.

### SUMMARY

In one aspect, the present disclosure provides a molecule which exhibits enhanced transport into the central nervous system. The molecule includes a modified IgG Fc region which comprises the transport-enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to EU numbering. The transport-enhancing amino acid substitutions enhance the transport of the molecule into the brain relative to a molecule comprising a non-modified IgG Fc region.

In some embodiments, the transport-enhancing amino acid substitutions consist of M252Y/V308P.

In some embodiments, the modified IgG Fc region further comprises one additional transport-enhancing amino acid substitution selected from the group consisting of S254T, T256D, T256E, T256H, T256L, T256N, T256P, T256Q, T256W, N434A, N434F, N434G, N434H, N434M, N434P, N434Q, N434R, N434S, and N434W, with amino acid residue numbering according to EU numbering. In one embodiment, the transport-enhancing amino acid substitutions consist of M252Y/V308P and one additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W, with amino acid residue numbering according to EU numbering.

In some embodiments, the modified IgG Fc region further comprises an additional two transport-enhancing amino substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y, with amino acid residue numbering according to EU numbering. In one embodiment, the transport-enhancing amino acid substitutions consist of M252Y/V308P and two transport-enhancing amino substitutions selected from the group consisting of S254A/N434Y, S254F/N434Y, S254G/N434Y, S254H/N434Y, S254T/T256E, S254T/N434W, T256A/N434F, T256A/N434S, T256A/N434W, S254T/N434Y, S254T/N434F, S254T/N434H, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y, with amino acid residue numbering according to EU numbering. In one embodiments, the additional two transport-enhancing amino acid substitutions are selected from the group selected from T256V/N434F; T256E/N434H; T256S/N434W; T256W/N434Y; T256E/N434F; T256R/N434Y; T256P/N434W; T256E/N434Y; T256F/N434F; T256W/N434W; T256F/N434Y; T256I/N434W; T256Q/N434W; T256E/N434W; T256A/N434W; T256E/N434P; T256V/N434W; T256I/N434Y; T256R/N434W; T256G/N434Y; T256I/N434Y; T256V/N434Y; T256Y/N434Y; T256N/N434Y; T256Q/N434Y; T256A/N434Y; T256P/N434Y; T256S/N434Y; T256D/N434W; and T256H/N434F, with amino acid residue numbering according to EU numbering.

In some embodiments, the modified IgG Fc region further comprises an additional three transport-enhancing amino substitutions selected from the group consisting of S254T/T256E/N434Y; S245T/T256E/N434F; and S254T/T256E/N434H, with amino acid residue numbering according to EU numbering.

In some embodiments, the transport-enhancing amino acid substitutions consist of M252Y, S254T, T256E, V308P, N434W.

In some embodiments, the modified IgG Fc is an IgG1 Fc. In one embodiment, the molecule further comprises one or more Fc modifications selected from the group consisting of L235A/G237A, L235E, L235E/P329G, a substitution to remove the glycosylation site at N297 such as N297A, A330S/P331S, L234A/L235A/P329G and K447Δ, with amino acid residue numbering according to EU numbering.

In some embodiments, the modified IgG Fc is an IgG2 Fc. In one embodiment, the molecule further comprises one or more Fc modifications selected from the group consisting of V235E, V235A/G237A, V235A/G237A, an aglycosylating substitution at N297 such as N297A, A330S/P331S, and K447Δ, with amino acid residue numbering according to EU numbering.

In some embodiments, the modified IgG Fc is an IgG4 Fc. In one embodiment, the molecule further comprises an additional alteration selected from S228P and/or one or more Fc modifications selected from the group consisting of L235E, L235A/G237A, L235E/P329G, an aglycosyating substitution at N297 such as N297A, P331S, and K447Δ, with amino acid residue numbering according to EU numbering.

In some embodiments, the amino acid sequence of the modified Fc region sequence comprises a sequence selected from SEQ ID NOs: 731-881.

In some embodiments, the molecule comprising the modified IgG Fc region comprises or consists of an antibody. In one embodiment, the molecule consists of an IgG1, an IgG2 or an IgG4 antibody.

In some embodiments, the molecule binds to an extracellular antigen and/or a cell surface antigen in the CNS. In one embodiment, the extracellular antigen and/or cell surface antigen is selected from the group consisting of beta-secretase 1 (BACE1), amyloid beta (Abeta), epidermal growth factor receptor (EGFR), human epidermal growth factor receptor 2 (HER2), tau, apolipoprotein E (ApoE), CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL 1 b), caspase 6, triggering receptor expressed on myeloid cells 2 (TREM2), Clq, paired immunoglobin like type 2 receptor alpha (PILRA), CD33, interleukin 6 (IL6), tumor necrosis factor alpha (TNFa), tumor necrosis factor receptor superfamily member 1A (TNFR1), tumor necrosis factor receptor superfamily member IB (TNFR2), apolipoprotein J (ApoJ), Tau protein (e.g., a human Tau protein) or a fragment thereof, phosphorylated Tau protein, an unphosphorylated Tau protein, a splice isoform of Tau protein, an N-terminal truncated Tau protein, a C-terminal truncated Tau protein, and/or a fragment thereof, and alpha-synuclein protein (e.g., a human alpha-synuclein protein) or a fragment thereof.

In some embodiments, the molecule demonstrates at least a 10-fold enhanced internalization in a JEG3-hFcRn cell internalization assay relative to a molecule comprising an Fc region that does not comprise blood-brain barrier enhancing substitutions.

Another aspect of the present disclosure provides a fusion protein which exhibits enhanced blood-brain barrier transport. The fusion protein comprises a modified IgG Fc region, wherein the modified IgG Fc region comprises the blood-brain barrier transport enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to EU numbering. The amino acid substitutions enhance the blood brain barrier transport of the molecule into the brain.

In some embodiments, the fusion protein further comprises an additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W.

In some embodiments, the fusion protein further comprises an additional transport-enhancing amino acid substitution selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256I/N434I, T256L/N434K, T256I/N434W, T256I/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y. In one embodiment, the additional amino acid substitutions are selected from the group selected from T256V/N434F; T256E/N434H; T256S/N434W; T256W/N434Y; T256E/N434F; T256R/N434Y; T256P/N434W; T256E/N434Y; T256F/N434F; T256W/N434W; T256F/N434Y; T256I/N434W; T256Q/N434W; T256E/N434W; T256A/N434W; T256E/N434P; T256V/N434W; T256I/N434Y; T256R/N434W; T256G/N434Y; T256I/N434Y; T256V/N434Y; T256Y/N434Y; T256N/N434Y; T256Q/N434Y; T256A/N434Y; T256P/N434Y; T256S/N434Y; T256D/N434W; and T256H/N434F.

In some embodiments, the fusion protein further comprises additional transport-enhancing amino acid substitution(s) selected from the group consisting of S254T/T256E/V308P/N434W; S254T/T256E/N434W; N286E/M428I/N434Y; N434W; T256E/N434W; T256E; S254T/T256E; and S254T/N434W.

In some embodiments, the modified IgG Fc region is fused to a therapeutic protein. In one embodiment, the therapeutic protein is an enzyme. In one embodiment, the enzyme is beta-glucuronidase or N-acetylglucosaminidase.

In some embodiments, the IgG Fc region is an IgG1 Fc region.

In some embodiments, the IgG Fc region is an IgG2 Fc region.

In some embodiments, the IgG Fc region is an IgG4 Fc region.

A further aspect of the present disclosure provides a method of enhancing delivery of a molecule comprising an IgG Fc region to the brain. The method includes modifying the amino acid sequence of the IgG Fc region to comprise the transport-enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering, wherein the amino acid substitutions enhance blood brain barrier transport and administering the molecule to the brain.

In some embodiments, the method further comprises modifying the amino acid sequence of the IgG Fc region to comprise an additional transport enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W. In one embodiment, the method further comprises modifying the amino acid sequence of the IgG Fc region to comprise additional two transport-enhancing amino acid substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y. In one embodiment, the additional two transport-enhancing amino acid substitutions are selected from the group selected from T256V/N434F; T256E/N434H T256S/N434W; T256W/N434Y; T256E/N434F; T256R/N434Y; T256P/N434W; T256E/N434Y; T256F/N434F; T256W/N434W; T256F/N434Y; T256I/N434W; T256Q/N434W; T256E/N434W; T256A/N434W; T256E/N434P; T256V/N434W; T256I/N434Y; T256R/N434W; T256G/N434Y; T256I/N434Y; T256V/N434Y; T256Y/N434Y; T256N/N434Y; T256Q/N434Y; T256A/N434Y; T256P/N434Y; T256S/N434Y; T256D/N434W; and T256H/N434F.

In some embodiments, the method further comprises modifying the amino acid sequence of the IgG Fc region to comprise one or more amino acid substitutions selected from the group consisting of S282P, an aglycosylating substitution at N297 such as N297A, A330S, P331S, and P329G.

In some embodiments, the modified IgG Fc is an IgG1 Fc.

In some embodiments, the modified IgG Fc is an IgG2 Fc.

In some embodiments, the modified IgG Fc is an IgG4 Fc.

In some embodiments, the molecule comprising the modified IgG Fc region comprises an antibody. In some embodiments, the molecule comprising the modified IgG Fc region consists of an antibody. In one embodiment, the antibody binds to a cell surface antigen in the CNS. In one embodiment, the cell surface antigen is selected from the group consisting of beta-secretase 1 (BACE1), amyloid beta (Abeta), epidermal growth factor receptor (EGFR), human epidermal growth factor receptor 2 (HER2), tau, apolipoprotein E (ApoE), CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL 1 b), caspase 6, triggering receptor expressed on myeloid cells 2 (TREM2), Clq, paired immunoglobin like type 2 receptor alpha (PILRA), CD33, interleukin 6 (IL6), tumor necrosis factor alpha (TNFa), tumor necrosis factor receptor superfamily member 1 A (TNFR1), tumor necrosis factor receptor superfamily member IB (TNFR2), apolipoprotein J (ApoJ), Tau protein (e.g., a human Tau protein) or a fragment thereof, phosphorylated Tau protein, an unphosphorylated Tau protein, a splice isoform of Tau protein, an N-terminal truncated Tau protein, a C-terminal truncated Tau protein, and/or a fragment thereof, and alpha-synuclein protein (e.g., a human alpha-synuclein protein) or a fragment thereof.

Another aspect of the disclosure provides a method of treating a disorder which is associated with the central nervous system. The method includes administering a molecule comprising a modified IgG Fc region wherein said modified IgG Fc region comprises the transport-enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering.

In one embodiment, the modified IgG Fc region further comprises an additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W. In one embodiment, the modified IgG Fc region further comprises an additional two transport-enhancing amino acid substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256L/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y. In one embodiment, the additional two transport-enhancing amino acid substitutions are selected from the group selected from T256V/N434F; T256E/N434H; T256S/N434W; T256W/N434Y; T256E/N434F; T256R/N434Y; T256P/N434W; T256E/N434Y; T256F/N434F; T256W/N434W; T256F/N434Y; T256I/N434W; T256Q/N434W; T256E/N434W; T256A/N434W; T256E/N434P; T256V/N434W; T256I/N434Y; T256R/N434W; T256G/N434Y; T256I/N434Y; T256V/N434Y; T256Y/N434Y; T256N/N434Y; T256Q/N434Y; T256A/N434Y; T256P/N434Y; T256S/N434Y; T256D/N434W; and T256H/N434F.

In some embodiments, the modified IgG Fc region further comprises one or more amino acid substitutions selected from the group consisting of S282P, G237A, an aglycosylating substitution at N297 such as N297A, A330S, P331S, and P329G.

In some embodiments, the modified IgG Fc is an IgG1 Fc.

In some embodiments, the modified IgG Fc is an IgG2 Fc.

In some embodiments, the modified IgG Fc is an IgG4 Fc.

In some embodiments, the molecule comprising the modified IgG Fc region comprises an antibody. In some embodiments, the molecule comprising the modified IgG Fc region consists an antibody. In some embodiments, the antibody binds to a cell surface antigen in the CNS. In some embodiments, the cell surface antigen is selected from the group consisting of beta-secretase 1 (BACE1), amyloid beta (Abeta), epidermal growth factor receptor (EGFR), human epidermal growth factor receptor 2 (HER2), tau, apolipoprotein E (ApoE), CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL 1 b), caspase 6, triggering receptor expressed on myeloid cells 2 (TREM2), Clq, paired immunoglobin like type 2 receptor alpha (PILRA), CD33, interleukin 6 (IL6), tumor necrosis factor alpha (TNFa), tumor necrosis factor receptor superfamily member 1 A (TNFR1), tumor necrosis factor receptor superfamily member IB (TNFR2), apolipoprotein J (ApoJ), Tau protein (e.g., a human Tau protein) or a fragment thereof, phosphorylated Tau protein, an unphosphorylated Tau protein, a splice isoform of Tau protein, an N-terminal truncated Tau protein, a C-terminal truncated Tau protein, and/or a fragment thereof, and alpha-synuclein protein (e.g., a human alpha-synuclein protein) or a fragment thereof.

In some embodiments, the disorder is selected from Sly syndrome and Sanfilippo syndrome.

The disclosure also provides use of a molecule comprising a modified IgG Fc region wherein said modified IgG Fc region comprises the transport-enhancing amino acid substitutions M252Y/V308P and one additional amino acid substitution selected from the group consisting of Y300Q, Y300T, Y300S, Y300K, Y300N, Y300P, Y300D, Y300G, Y300H R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering, for the manufacture of a medicament for treating a disorder which is associated with the central nervous system.

Also provided herein is a molecule comprising a modified IgG Fc region wherein said modified IgG Fc region comprises the transport-enhancing amino acid substitutions M252Y/V308P and one additional amino acid substitution selected from the group consisting of Y300Q, Y300T, Y300S, Y300K, Y300N, Y300P, Y300D, Y300G, Y300H R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering, for use in treating a disorder which is associated with the central nervous system.

Each of the aspects and embodiments described herein are capable of being used together, unless excluded either explicitly or clearly from the context of the embodiment or aspect.

### BRIEF DESCRIPTION OF THE FIGURES

FIGS. 1A-1C show an example of HALO quantification of human IgG-kappa immunohistochemistry staining in a brain from a mouse which had been administered with antibody variant targeting O4 and bearing the M252Y/T256E/V308P substitutions. First, an annotation contour is drawn around the section (FIG. 1A). Then, the HALO tissue classifier module was applied within the contour (FIG. 1B). Finally, areas of weak, moderate, or strong staining were identified by the algorithm (FIG. 1C).

FIG. 2A shows a sequence alignment of the heavy chain constant regions of wild type human IgG1, IgG2 and IgG4 numbered according to the EU numbering scheme. The highlights identify amino acid residues in IgG2 and IgG4 which differ from human IgG1. A dash (-) identifies a residue which is absent from a sequence relative to the corresponding IgG1 sequence. Dashed, solid, and dotted lines above the residue numbers indicate the CH1, CH2, and CH3 regions respectively. The Fc region is the region spanning the CH2 and CH3 regions. FIG. 2B provides a diagrammatic representation of the structure of an IgG antibody protein with the variable, constant, and Fc regions highlighted.

FIG. 3 shows a graph which plots the degree of correlation between the results of brain immunohistochemistry studies (as described in Examples 2 and 3, in optical density units on the Y-axis) and the antibody internalization assay (as described in Example 4, as percent internalization relative to M252Y/S254T/T256E/V308P/N434W on the X-axis) for a variety of different variant human antibody sequences. Each dot represents one of the 80 variants which were assayed both by brain immunohistochemistry in Tables 5 and 6 as well as by the antibody internalization assay in Table 7. Some variants whose values on the Y or X axes were not clustered near the origin are labeled. The line of best fit has a positive slope. The correlation coefficient of the data was 0.43 and was significantly different from zero (p<0.0001).

FIGS. 4A-4C shows exemplary micrographs of pERK staining in Tg276 mice after the administration of the following hIgG1 Fc-modified variants as described in Example 7: FIG. 4A anti-TrkB-hIgG1; FIG. 4B anti-KLH-hIgG1 (isotype control); and FIG. 4C anti-TrkB-hIgG1-M252Y/S254T/T256E/V308P/N434W.

FIG. 5A is a graph quantifying human IgG-kappa immunostaining in whole brain of adult male Tg276 mice as described in Example 7. FIG. 5B is a graph quantifying human IgG-kappa immunostaining in the hippocampus region of the same adult male Tg276 mice shown in FIG. 5A and as described in Example 7. FIG. 5C shows graphs quantifying pERK1/2 immunostaining intensity (excluding CA3) on the left and cell number in the hippocampus region on the right in adult male Tg276 mice as shown in FIG. 4 and as described in Example 7. For each graph column A represents immunostaining of mice dosed with an anti-TrkB antibody formatted onto a human IgG1 isotype, column B represents immunostaining of mice dosed with an anti-Keyhole Limpet Haemocyanin antibody (KLH, not expressed in the brain) formatted onto a human IgG1 isotype, and column C represents immunostaining of mice dosed with the anti-TrkB antibody formatted onto a human IgG1 with the substitutions M252Y/S254T/T256E/V308P/N434W.

FIG. 6A is a graph comparing pharmacokinetics in the brains of adult male Tg276 mice following the administration of anti-O4 antibody with either wild-type or with M252Y/S254T/T256E/V308P/N434W substitutions. FIG. 6B is a graph comparing pharmacokinetics in the serum of the adult male Tg276 mice as shown in FIG. 6A. FIG. 6C is a graph comparing pharmacokinetics in the CSF of the adult male Tg276 mice as shown in FIGS. 6A and 6B.

FIG. 7 shows a graph which plots the degree of correlation between the results of the transcytosis assay (as described in Example 9) as concentration relative to the IAHA antibody (on the X-axis) against the amount of antibody detected in the brain by immunohistochemistry (as described in Examples 2 and 3), in optical density units (on the Y-axis) for a range of different variant human antibody sequences. The dots represent each of the variants which were assayed both by the transcytosis assay of Table 10 as well as by brain immunohistochemistry of Tables 5, 6 and 8. The correlation coefficient of the data was 0.02 and was not significantly different from zero (p=0.92).

FIGS. 8A-8B plots the degree of correlation between the K_{D} of antibody-FcRn interactions at pH 6.0 and pH 7.4. Each graph plots the K_{D} of the antibody variants tested in Example 10 and the brain immunohistochemistry results on Tables 5, 6, and 8. FIG. 8A shows the K_{D} (in M) at pH 6.0 of each variant plotted on the horizontal axis on a logarithmic scale and the integrated optical density of the brain immunohistochemistry assay for each variant plotted on the vertical axis. The correlation coefficient of the data was 0.02 and was not significantly different from zero (p=0.94). (FIG. 8B) shows the K_{D} (in M) at pH 7.4 of each variant plotted on the horizontal axis on a logarithmic scale and the integrated optical density of the brain immunohistochemistry assay for each variant plotted on the vertical axis. The correlation coefficient of the data was 0.30 and was not significantly different from zero (p=0.20).

### DETAILED DESCRIPTION

The present disclosure relates to molecules comprising one or more modified IgG Fc regions that enhance transport of the molecule into the brain parenchyma. As used herein, when describing the enhanced transport of a molecule comprising a modified Fc region into the brain it is contemplated that there is an increase in transport across one or more of the barriers making up the blood-brain barrier into the brain, such that the total amount of molecule entering the brain increases relative to a corresponding molecule comprising an unmodified Fc region. Accordingly, in certain embodiments the present disclosure provides an antibody comprising at least one modified IgG Fc region or a molecule comprising at least one modified IgG Fc region that, for example, may be used to transport a therapeutic moiety across the blood-brain barrier, to be taken up by the brain. Alternatively, the present disclosure provides molecules comprising modified IgG Fc regions for use in transporting one or more compounds across the BBB.

The present disclosure also provides compositions and methods useful for producing molecules comprising a modified IgG Fc region, methods of enhancing the transport of a molecule into the brain, fusion proteins comprising a modified Fc region, nucleic acids encoding the same, as well as methods for the treatment or prevention of various health conditions associated with disorders within the central nervous system such as various neurological disorders.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

“Blood-brain barrier” or “BBB” refers to the physiological barriers between the peripheral circulation and the brain, the retina and the spinal cord (i.e., the central nervous system (CNS)) which is formed in part by tight junctions within the brain capillary endothelial plasma membranes, creating a tight barrier that restricts the transport of molecules into the brain, even very small molecules such as urea (60 Daltons). The blood-brain barrier within the brain, the blood-spinal cord barrier within the spinal cord, and the blood-retinal barrier within the retina are contiguous capillary barriers within the CNS, and are herein collectively referred to the blood-brain barrier or BBB. The BBB also encompasses the blood-CSF barrier (choroid plexus) where the barrier is comprised of ependymal cells rather than capillary endothelial cells.

The term “Fc region” or “Fc” herein is used to define a C-terminal region of an IgG heavy chain that comprises at least both CH2 and CH3 heavy chain constant domains. The term includes native sequence Fcs and modified Fcs. The Fc region may be either an IgG1 Fc region, an IgG2 Fc region or an IgG4 Fc region. The Fc region may be part of an antibody, or it may consist of only the CH2/CH3 domains of an antibody.

The term “modified Fc region” as used herein relates to an IgG Fc region which comprises the transport-enhancing amino acid substitutions M252Y/V308P at least. The modified Fc region may comprise one, two, three or additional transport-enhancing amino acid substitutions in combination with the M252Y/V308P transport-enhancing substitutions, as described herein. The modified Fc region may also comprise other amino acid substitutions or deletions which are not transport-enhancing, provided the Fc region comprises at least one set of transport-enhancing amino acid substitutions described herein. Similarly, a “non-modified” Fc region is one that lacks transport-enhancing amino acid substitutions, but which may comprise other non-transport-enhancing amino acid substitutions or deletions when compared to a native Fc sequence. Typically, such non-modified Fc regions comprises a native IgG Fc amino acid sequence at residues where transport-enhancing substitutions could be made.

As used herein, the term “antigen binding region” shall be taken to mean a region of an antibody that is capable of specifically binding to an antigen, i.e., a V_{H} or a V_{L} or an F_{V} comprising both a V_{H} and a V_{L}.

The term “enhances”, “enhanced”, and like terms, in the context of “enhances transport” refers to an increase in transport of a molecule comprising a modified IgG Fc region into the brain that is at least 10%, 15%, 20%, 25%, 30%, 35%, 40%, 45%, 50%, 55%, 60%, 65%, 70%, 75%, 80%, 90%, 100%, 150%, 200%, 300%, 400%, 500%, 1000% or greater when compared to the transport of the molecule comprising a non-modified IgG Fc region. Methods to quantify transport of a molecule into the brain are described herein and include for instance the method of Immunostaining for Human IgG Kappa Light Chain in Mouse Brains described herein, the Cell Internalization assay described herein, or immunostaining for a downstream signal of molecular transport, such as the Immunostaining for pERK in Mouse Brains described herein. In one embodiment the enhancement of transport of a molecule comprising a modified IgG Fc region is measured by comparing the transport of the molecule comprising a modified Fc region with the transport of a molecule which is the same apart from having a “non-modified” Fc region, that is the Fc region of the comparator molecule lacks the transport-enhancing amino acid substitutions. In another embodiment the enhancement of transport of a molecule comprising a modified IgG Fc region is measured by comparing the transport of the molecule comprising a modified Fc region with the transport of a molecule which is the same apart from having an Fc region comprising the transport-enhancing substitutions M252Y/V308P only.

“Binding” generally refers to the strength of the sum total of noncovalent interactions between a single binding site of a molecule (e.g., an antibody) and its binding partner (e.g., an antigen/target, or between an Fc region and an Fc receptor such as an FcRn). Unless indicated otherwise, as used herein, “binding” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure, an example of which is an affinity ELISA assay. In addition, affinity can be determined by a surface plasmon resonance assay (SPR, e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). Binding affinity can be also determined by another kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹)¹) can be measured. The dissociation rate constant (k_{d} in s⁻¹) can be calculated from these values K_{D}×kₐ). Binding affinity can be also determined by an equilibrium/solution method.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

The terms “administration” and “administering”, as used herein, refer to the delivery of a composition or formulation as disclosed herein by an administration route including, but not limited to, intravenous, intra-arterial, intracranial, intramuscular, intraperitoneal, subcutaneous, intramuscular, or combinations thereof. The term includes, but is not limited to, administration by a medical professional and self-administration.

As used herein, a “subject” or an “individual” includes mammals, such as human (e.g., human individuals) and non-human mammals. In some embodiments, a “subject” or “individual” is a patient under the care of a physician. Thus, the subject can be a human patient who has, is at risk of having, or is suspected of having a disease of interest and/or one or more symptoms of the disease. The subject can also be an individual who is diagnosed with a risk of the condition of interest at the time of diagnosis or later. For example, the subject can be further characterized as being at risk of developing a condition described herein.

The terms “cell”, “cell culture” and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The term “comprising” is synonymous with “including”, “containing”, or “characterized by”, and is inclusive or open-ended and does not exclude additional, unrecited elements or method steps. Any recitation herein of the term “comprising”, particularly in a description of components of a composition or in a description of steps of a method, is understood to encompass those compositions and methods consisting essentially of and consisting of the recited components or steps. As used herein, “consisting of” excludes any elements, steps, or ingredients not specified in the claimed composition or method.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions

In certain embodiments the present disclosure provides molecules which comprise a modified IgG Fc region wherein the modified IgG Fc region comprises the transport-enhancing amino acid substitutions M252Y/V308P, with amino acid residue numbering according to the EU numbering.

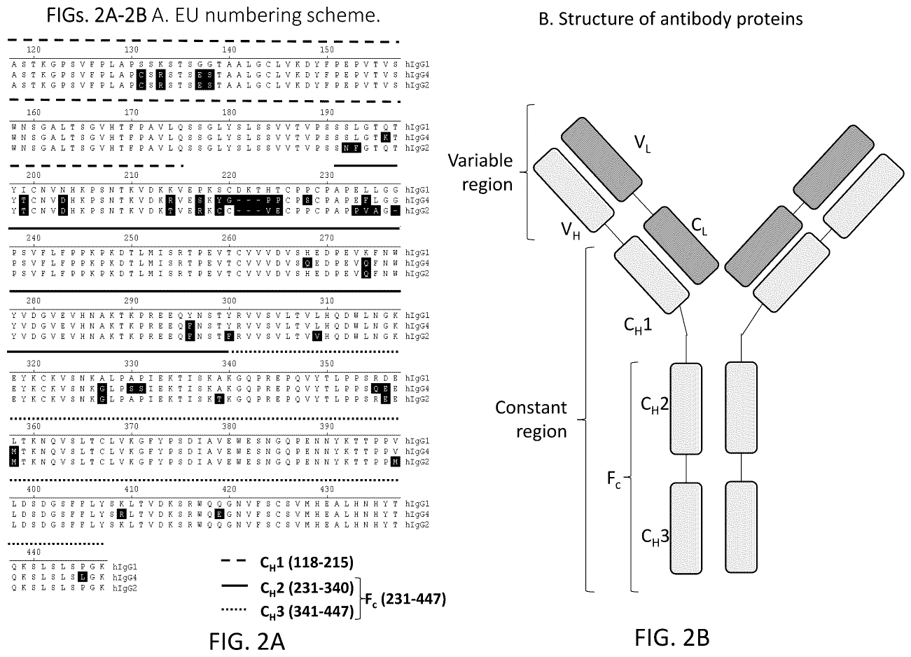

As used herein, “modified” refers to the introduction of amino acid substitutions in the Fc region that modulate transport activity into tissues of the central nervous system protected by the blood-brain barrier, such as the brain, the spinal cord and the retina. Unless otherwise specified herein, numbering of amino acid residues in the heavy chain constant regions is according to the EU numbering system, also called the EU index, as described in Kabat et al., Sequences of Proteins of Immunological Interest, 5th Ed. Public Health Service, National Institutes of Health, Bethesda, MD, 1991 and as set out in FIG. 2.

As used herein, and as described supra, an Fc could be a single heavy chain Fc, a paired heavy chain Fc, or multiple Fc regions on a single molecule, provided that the Fc region retains the ability to bind to an FcRn. The molecules provided herein comprise at least one modified Fc region which has been engineered to enhance their ability to be transported into the central nervous system, such as the brain.

In some embodiments, the modified Fc region further comprises, in addition to the transport-enhancing amino acid substitutions M252Y/V308P, an additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W. Thus, in addition to comprising the M252Y/V308P amino acid substitutions, the modified Fc region comprises one additional transport-enhancing amino acid substitutions selected from the above list. In some embodiments the modified Fc region comprises transport-enhancing amino acid substitutions which consist of M252Y/V308P and one additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W.

In some embodiments, the modified IgG Fc region further comprises, in addition to the transport-enhancing amino acid substitutions M252Y/V308P, an additional two transport-enhancing amino substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y. In some embodiments the modified Fc region comprises transport-enhancing amino acid substitutions which consist of M252Y/V308P and two additional transport-enhancing amino acid substitution selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y.

In some embodiments, the modified IgG Fc region further comprises, in addition to the transport-enhancing amino acid substitutions M252Y/V308P, an additional three transport-enhancing amino substitutions selected from the group consisting of S254T/T256E/N434Y; S245T/T256E/N434F; and S254T/T256E/N434H, with amino acid residue numbering according to EU numbering. In some embodiments the modified Fc region comprises transport-enhancing amino acid substitutions which consist of M252Y/V308P and three additional transport-enhancing amino acid substitution selected from the group consisting of S254T/T256E/N434Y; S245T/T256E/N434F; and S254T/T256E/N434H.

In one embodiment the molecule comprising the modified Fc region described herein is an antibody that further comprises one or more antigen binding domains which target at least one antigen present in the central nervous system (CNS), with the modified Fc region targeting the antibody to the central nervous system by exhibiting an enhanced ability to be transported into the brain when compared to a molecule comprising an unmodified Fc region.

As demonstrated in the Examples, the introduction of multiple substitutions into the same Fc region can in some cases result in an unexpected synergistic increase in transport into the brain parenchyma relative to a corresponding unsubstituted Fc region, whilst other substitutions do not change or even reduce transport relative to a corresponding unsubstituted Fc region. Thus, the molecules provided herein achieve multi-fold increases in transport into the central nervous system, such as the brain, for example as measured in an antibody internalization assay as described herein.

Also provided, in other related aspects of the disclosure, are fusion proteins comprising a modified IgG Fc region comprising transport-enhancing amino acid substitutions that enhances transport of the molecule into the brain relative to a fusion protein comprising an IgG Fc of a corresponding isotype which does not comprise the described transport-enhancing substitutions.

### Modified IgG Fc Regions

The present disclosure is based, inter alia, on the recognition that molecules which comprise a modified IgG Fc region with specific transport-enhancing amino acid substitutions exhibit increased transport into the brain when compared to a molecule comprising an unmodified Fc region. The molecules described herein comprise a modified IgG Fc region, wherein said modified IgG Fc region comprises the transport enhancing amino acid substitutions M252Y/V308P, with amino acid residue numbering according to the EU numbering. The combination of the two substitutions M252Y/V308P enhance transport of the Fc region into the central nervous system, such as the brain, relative to an unmodified Fc region. The relative level of transport of a modified Fc region or a molecule comprising the modified Fc region into the brain compared to an unmodified Fc region may be quantified in a model of antibody internalization as described herein or in a Immunostaining for Human IgG Kappa Light Chain in the humanized Mouse Brain model as described herein.

The Fc regions described herein are human origin Fc regions unless described to the contrary. For all molecules provided herein, the constant domain amino acid residue numbering is according to the “EU numbering system” (Edelman G M et al., Proc Natl Acad Sci USA, 63(1):78-85 (1969)). As described herein, when multiple amino acid substitutions are described in the Fc region, each individual substitution is separated by a “/”, with the original residue (in single letter amino acid format)-residue number according to EU numbering-new residue (in single letter amino acid format).

The Fc regions described herein define a C-terminal region of an immunoglobulin heavy chain that comprises at least heavy chain constant domains CH2 and CH3, or a portion of heavy chain constant domains CH2 and CH3 which is able to bind the FcRn. The term Fc includes native sequence (wild-type) Fcs and modified Fcs. In some embodiments, a human IgG heavy chain Fc extends from C226, or from P230 (numbering according to EU numbering), to the carboxyl-terminus of the antibody heavy chain constant region. The C-terminal lysine (K447) of the Fc may be present or absent. Commonly the K447 residue is deleted from the encoding polynucleotide (ΔK447) in order to reduce the heterogeneity of the antibody heavy chain when expressed in an antibody manufacturing context. In some embodiments, the molecule comprising an Fc also comprises an antibody hinge region. In some embodiments, the molecule comprising an Fc also comprises a heavy chain constant domain CH1 and an antibody hinge region.

In some embodiments, the molecule of the present disclosure which comprises a modified IgG Fc comprises or consists of an antibody. An antibody as used herein has its common meaning in the field and refers to an immunoglobulin molecule that recognizes and specifically binds to an epitope of a target through at least one antigen binding domain within the variable region of the immunoglobulin molecule. The target can be a peptide or polypeptide or glycopolypeptide, e.g., an extracellular or cell surface peptide in the CNS. An antibody of this disclosure encompasses full length antibodies (including full length polyclonal antibodies and full length monoclonal antibodies), multispecific antibodies such as bispecific antibodies generated from at least two full length antibodies, chimeric antibodies, humanized antibodies, human antibodies, fusion proteins comprising an antigen-binding portion of an antibody and an Fc region, and any other modified immunoglobulin molecule comprising an antigen recognition site and modified Fc region so long as the antibodies exhibit the desired biological activity. An antibody can be of the IgG subclasses (isotypes) of IgG1, IgG2, or IgG4. In some embodiments, the modified IgG Fc of the present disclosure is comprised within an IgG antibody. In certain embodiments, the modified IgG Fc of the present disclosure is an IgG1 Fc. In other embodiments, the modified IgG Fc of the present disclosure is a modified IgG2 Fc. In some embodiments, the modified IgG Fc of the present disclosure is a modified IgG4 Fc. The different classes of immunoglobulins have different and well-known amino acid sequences (as set out in FIG. 2A), subunit structures and three-dimensional configurations.

When the molecule of the present disclosure is an antibody, the antibody may comprise one or more variable regions. A variable region of an antibody refers to the variable region of the antibody light chain (VL) or the variable region of the antibody heavy chain (VH), either alone or in combination. The variable regions of the heavy and light chain each consist of four framework regions (FR) connected by and alternating with three complementarity determining regions (CDRs). The CDRs in each chain are held together in close proximity by the FRs and, with the CDRs from the other chain, contribute to the formation of the antigen-binding site of antibodies.

In some embodiments, the molecules provided herein are full length antibodies. A full-length antibody can include a four polypeptide unit consisting of two heavy chains and two light chains, as described in greater detail below, held together by disulfide bonds. The light chains are generally shorter, with lower molecular weights than the heavy chains. Each polypeptide chain has a constant region and a variable region. The variable region is specific to each particular antibody. The light chain variable region is referred to as VL and the light chain constant region as CL. Similarly, the heavy chain variable region is referred to as VH and the heavy chain constant regions as CH, with CH1, CH2, and CH3 each denoting a different domain of the constant region of the heavy chain. In some embodiments, carbohydrates can be normally attached to the CH2 domains of the heavy chains. Further, a full-length antibody contains an Fc region. The Fc region contains only constant regions from the heavy chains (CH).

As described above, the antibody of the present disclosure may include one or more constant regions. A “constant region” of an antibody is a well-known term in the art and refers to the part of the antibody that is relatively constant in amino acid sequence between different molecules. Typically, the heavy chain constant region is composed of three distinct domains, termed CH1, CH2, and CH3, numbered in the direction from the amino terminal (N-terminal) end to the carboxy terminal (C-terminal) end. A typical light chain only has one constant region domain, termed CL. The heavy chain constant region of an antibody determines its particular effector function. One of skill in the art will readily understand the terminology and structural features of constant regions of antibodies.

The term “epitope” refers to that portion of an antigen capable of being recognized and specifically bound by a particular antibody. When the antigen is a polypeptide, epitopes can be formed both from contiguous amino acids and non-contiguous amino acids juxtaposed by tertiary folding of a protein. Epitopes formed from contiguous amino acids are typically retained upon protein denaturing, whereas epitopes formed by tertiary folding are typically lost upon protein denaturing.

An antibody provided herein can be a monoclonal antibody. As used herein, “monoclonal antibody” refers to an antibody obtained from a substantially homogeneous population of antibodies, i.e., the individual antibodies that make up that population are naturally occurring variants that may be present in trace amounts. Each monoclonal antibody typically targets a single determinant on the antigen, in contrast to polyclonal antibody preparations, which typically include a range of different antibodies that target different determinants (epitope). The modifier “monoclonal” indicates the nature of the antibody as being obtained from a substantially homogeneous population of antibodies and should not be construed as requiring the production of the antibody by any particular method. The term “monoclonal antibody” encompasses full length monoclonal antibodies, fusion proteins comprising an antibody portion, and any other modified immunoglobulin molecule comprising an antigen recognition site and an Fc region. Furthermore, “monoclonal antibody” refers to such antibodies made in a variety of manners including but not limited to hybridoma, phage selection, recombinant expression, and transgenic animals.

The antibodies encompassed by the present disclosure can be human, non-human, humanized, chimeric, or resurfaced. In certain embodiments the antibodies are human or humanized.

In certain embodiments, an antibody provided herein is a multispecific antibody, e.g., a bispecific antibody. Multispecific antibodies are monoclonal antibodies that have binding specificities for at least two different epitopes. In certain embodiments, bispecific antibodies may bind to two different epitopes of the same antigen. In certain embodiments, bispecific antibodies may bind to two different antigens. Bispecific antibodies can be prepared as full-length antibodies or antibody fragments, as long as the antibody fragment comprises a modified Fc region as described herein.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al, EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et ah, Science, 229: 81 (1985)); or using leucine zippers to produce bi-specific antibodies (see, e.g., Kostelny et al, J. Immunol., 148(5): 1547-1553 (1992));

Engineered antibodies with three or more functional antigen binding sites, including “Octopus antibodies” or “dual-variable domain immunoglobulins” (DVDs) are also included herein (see, e.g., US 2006/0025576A1, and Wu et al. Nature Biotechnology (2007)).

### Exemplary Fc Regions

As discussed supra, the modified Fc region can be of any the following human IgG isotypes: IgG1, IgG2, and IgG4. The Fc region of the molecules of the present disclosure comprise a CH2 and a CH3 constant region. Exemplary sequences of some Fc regions of that can be modified to create a molecule of the present disclosure are provided below in Table 1. The constant region comprises a modified Fc region, for example Fc regions that can be modified in accordance with the present disclosure are described in Table 1 (see, e.g., SEQ ID Nos: 529, 565, 657-660). For example, Fc region sequences or CH1-CH3 domain sequences that are listed herein in Table 1 can be modified with transport-enhancing amino acid substitutions as described herein. In one embodiment, the Fc region is based on a human IgG1 sequence (i.e., SEQ ID NO:529). In one embodiment the Fc region is based on a human IgG2 sequence (i.e., SEQ ID NO:657). In one embodiment the Fc region is based on a human IgG4 sequence (i.e., SEQ ID NO:659).

In some embodiments, the Fc region of the molecules of the present disclosure can be an Fc region or CH1-CH3 domains as described in Table 1 that comprises non-transport enhancing amino acid substitutions, additions, or deletions (see, e.g., SEQ ID Nos 661-695). For example, constant regions of IgG can be any of the following: human IgG1 LAGA (L235A/G237A), human IgG1 YTE (M252Y/S254T/T256E), and variants and combinations thereof as shown in Table 1. In one embodiment, the Fc region or CH1-CH3 domains are based on a human IgG1 sequence (i.e., SEQ ID NO:529). In one embodiment the Fc region or CH1-CH3 domains are based on a human IgG2 sequence (i.e., SEQ ID NO:657). In one embodiment the Fc region or CH2-CH3 domains are based on a human IgG4 sequence (i.e., SEQ ID NO:659). In some embodiments, the modified Fc region of the molecules of the present disclosure can be an Fc fragment of the IgG4 constant region as described in Table 1. For example, modified constant region of IgG4 can be, e.g., human IgG4 S228P (SEQ ID NO: 20) and variants thereof.

In still other embodiments, the molecule of the present disclosure comprises any of the above modified Fc regions, such as an Fc region that is described in Table 1, wherein the Fc region sequence may further comprise a C-terminal Lysine (K) at position 447 according to EU numbering.

In some embodiments, the heavy chain constant region can be paired with light chain human constant regions as shown in Table 2.

TABLE 1
HUMAN Fc AND HEAVY CHAIN CONSTANT REGION SEQUENCES

| | | SEQ |
|---|---|---|
| Sequence | | ID |
| name | Sequence | NO |
| human IgG1 | APELLGGPSVFLFPPKPKDTLMISRTPEVTCVVVDVSHEDPEVKFNWYVDGVEVHN | 529 |
| Fc fragment | AKTKPREEQYNSTYRVVSVLTVLHQDWLNGKEYKCKVSNKALPAPIEKTISKAKGQP | |
| | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| Human IgG1 | ASTKGPSVFPLAPSSKSTSGGTAALGCLVKDYFPEPVTVSWNSGALTSGVHTFPAVL | 565 |
| CH1-CH3 | QSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| human IgG2 | APPVAGPSVFLFPPKPKDTLMISRTPEVTCVVVDVSHEDPEVQFNWYVDGVEVHN | 657 |
| Fc fragment | AKTKPREEQFNSTFRVVSVLTVVHQDWLNGKEYKCKVSNKGLPAPIEKTISKTKGQP | |
| | REPQVYTLPPSREEMTKNQVSLTCLVKGFYPSDIAVEWESNGQPENNYKTTPPMLD | |
| | SDGSFFLYSKLTVDKSRWQQGNVFSCSVMHEALHNHYTQKSLSLSPGK | |
| Human IgG2 | ASTKGPSVFPLAPCSRSTSESTAALGCLVKDYFPEPVTVSWNSGALTSGVHTFPAVL | 658 |
| CH1-CH3 | QSSGLYSLSSVVTVPSSNFGTQTYTCNVDHKPSNTKVDKTVERKCCVECPPCPAPPV | |
| | AGPSVFLFPPKPKDTLMISRTPEVTCVVVDVSHEDPEVQFNWYVDGVEVHNAKTKP | |
| | REEQFNSTFRVVSVLTVVHQDWLNGKEYKCKVSNKGLPAPIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPGK | |
| human IgG4 | APEFLGGPSVFLFPPKPKDTLMISRTPEVTCVVVDVSQEDPEVQFNWYVDGVEVHN | 659 |
| Fc fragment | AKTKPREEQFNSTYRVVSVLTVLHQDWLNGKEYKCKVSNKGLPSSIEKTISKAKGQP | |
| | REPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLGK | |
| Human IgG4 | ASTKGPSVFPLAPCSRSTSESTAALGCLVKDYFPEPVTVSWNSGALTSGVHTFPAVL | 660 |
| CH1-CH3 | QSSGLYSLSSVVTVPSSSLGTKTYTCNVDHKPSNTKVDKRVESKYGPPCPSCPAPEFL | |
| | GGPSVFLFPPKPKDTLMISRTPEVTCVVVDVSQEDPEVQFNWYVDGVEVHNAKTK | |
| | PREEQFNSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Human IgG4 | ASTKGPSVFPLAPCSRSTSESTAALGCLVKDYFPEPVTVSWNSGALTSGVHTFPAVL | 661 |
| S228P | QSSGLYSLSSVVTVPSSSLGTKTYTCNVDHKPSNTKVDKRVESKYGPPCPPCPAPEFL | |
| CH1-CH3 | GGPSVFLFPPKPKDTLMISRTPEVTCVVVDVSQEDPEVQFNWYVDGVEVHNAKTK | |
| | PREEQFNSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Human IgG1 | APELEGGPSVFLFPPKPKDTLMISRTPEVTCVVVDVSHEDPEVKFNWYVDGVEVHN | 662 |
| L235E | AKTKPREEQYNSTYRVVSVLTVLHQDWLNGKEYKCKVSNKALPAPIEKTISKAKGQP | |
| Fc fragment | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| Human IgG2 | APPEAGPSVFLFPPKPKDTLMISRTPEVTCVVVDVSHEDPEVQFNWYVDGVEVHNA | 663 |
| V235E | KTKPREEQFNSTFRVVSVLTVVHQDWLNGKEYKCKVSNKGLPAPIEKTISKTKGQPR | |
| Fc fragment | EPQVYTLPPSREEMTKNQVSLTCLVKGFYPSDIAVEWESNGQPENNYKTTPPMLDS | |
| | DGSFFLYSKLTVDKSRWQQGNVFSCSVMHEALHNHYTQKSLSLSPGK | |
| Human IgG4 | APEFEGGPSVFLFPPKPKDTLMISRTPEVTCVVVDVSQEDPEVQFNWYVDGVEVHN | 664 |
| L235E | AKTKPREEQFNSTYRVVSVLTVLHQDWLNGKEYKCKVSNKGLPSSIEKTISKAKGQP | |
| Fc fragment | REPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLGK | |
| Human IgG1 | APELAGAPSVFLFPPKPKDTLMISRTPEVTCVVVDVSHEDPEVKFNWYVDGVEVHN | 665 |
| L235A/G237A | AKTKPREEQYNSTYRVVSVLTVLHQDWLNGKEYKCKVSNKALPAPIEKTISKAKGQP | |
| Fc fragment | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| Human IgG4 | APEFAGAPSVFLFPPKPKDTLMISRTPEVTCVVVDVSQEDPEVQFNWYVDGVEVHN | 666 |
| L235A/G237A | AKTKPREEQFNSTYRVVSVLTVLHQDWLNGKEYKCKVSNKGLPSSIEKTISKAKGQP | |
| Fc fragment | REPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLGK | |
| Human IgG2 | APPAAAPSVFLFPPKPKDTLMISRTPEVTCVVVDVSHEDPEVQFNWYVDGVEVHN | 667 |
| V235A/G237A | AKTKPREEQFNSTFRVVSVLTVVHQDWLNGKEYKCKVSNKGLPAPIEKTISKTKGQP | |
| Fc fragment | REPQVYTLPPSREEMTKNQVSLTCLVKGFYPSDIAVEWESNGQPENNYKTTPPMLD | |
| | SDGSFFLYSKLTVDKSRWQQGNVFSCSVMHEALHNHYTQKSLSLSPGK | |
| Human IgG1 | APELLGGPSVFLFPPKPKDTLMISRTPEVTCVVVDVSHEDPEVKFNWYVDGVEVHN | 668 |
| N297A | AKTKPREEQYASTYRVVSVLTVLHQDWLNGKEYKCKVSNKALPAPIEKTISKAKGQP | |
| Fc fragment | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| Human IgG2 | APPVAGPSVFLFPPKPKDTLMISRTPEVTCVVVDVSHEDPEVQFNWYVDGVEVHN | 669 |
| N297A | AKTKPREEQFASTFRVVSVLTVVHQDWLNGKEYKCKVSNKGLPAPIEKTISKTKGQP | |
| Fc fragment | REPQVYTLPPSREEMTKNQVSLTCLVKGFYPSDIAVEWESNGQPENNYKTTPPMLD | |
| | SDGSFFLYSKLTVDKSRWQQGNVFSCSVMHEALHNHYTQKSLSLSPGK | |
| Human IgG4 | APEFLGGPSVFLFPPKPKDTLMISRTPEVTCVVVDVSQEDPEVQFNWYVDGVEVHN | 670 |
| N297A | AKTKPREEQFASTYRVVSVLTVLHQDWLNGKEYKCKVSNKGLPSSIEKTISKAKGQPR | |
| Fc fragment | EPQVYTLPPSQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |
| Human IgG1 | APELLGGPSVFLFPPKPKDTLMISRTPEVTCVVVDVSHEDPEVKFNWYVDGVEVHN | 671 |
| A330S/P331S | AKTKPREEQYNSTYRVVSVLTVLHQDWLNGKEYKCKVSNKALPSSIEKTISKAKGQP | |
| Fc fragment | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| Human IgG2 | APPVAGPSVFLFPPKPKDTLMISRTPEVTCVVVDVSHEDPEVQFNWYVDGVEVHN | 672 |
| A330S/P331S | AKTKPREEQFNSTFRVVSVLTVVHQDWLNGKEYKCKVSNKGLPSSIEKTISKTKGQP | |
| Fc fragment | REPQVYTLPPSREEMTKNQVSLTCLVKGFYPSDIAVEWESNGQPENNYKTTPPMLD | |
| | SDGSFFLYSKLTVDKSRWQQGNVFSCSVMHEALHNHYTQKSLSLSPGK | |
| Human IgG1 | APELEGGPSVFLFPPKPKDTLMISRTPEVTCVVVDVSHEDPEVKFNWYVDGVEVHN | 673 |
| L235E/P329G | AKTKPREEQYNSTYRVVSVLTVLHQDWLNGKEYKCKVSNKALGAPIEKTISKAKGQP | |
| Fc fragment | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| Human IgG4 | APEFEGGPSVFLFPPKPKDTLMISRTPEVTCVVVDVSQEDPEVQFNWYVDGVEVHN | 674 |
| L235E/P329G | AKTKPREEQFNSTYRVVSVLTVLHQDWLNGKEYKCKVSNKGLGSSIEKTISKAKGQP | |
| Fc fragment | REPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLGK | |
| Human IgG2 | APPEAGPSVFLFPPKPKDTLMISRTPEVTCVVVDVSHEDPEVQFNWYVDGVEVHNA | 675 |
| V235E/P329G | KTKPREEQFNSTFRVVSVLTVVHQDWLNGKEYKCKVSNKGLGAPIEKTISKTKGQPR | |
| Fc fragment | EPQVYTLPPSREEMTKNQVSLTCLVKGFYPSDIAVEWESNGQPENNYKTTPPMLDS | |
| | DGSFFLYSKLTVDKSRWQQGNVFSCSVMHEALHNHYTQKSLSLSPGK | |
| Human IgG1 | APELLGGPSVFLFPPKPKDTLMISRTPEVTCVVVDVSHEDPEVKFNWYVDGVEVHN | 676 |
| K4474 | AKTKPREEQYNSTYRVVSVLTVLHQDWLNGKEYKCKVSNKALPAPIEKTISKAKGQP | |
| Fc fragment | REPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| Human IgG2 | APPVAGPSVFLFPPKPKDTLMISRTPEVTCVVVDVSHEDPEVQFNWYVDGVEVHN | 677 |
| K4474 | AKTKPREEQFNSTFRVVSVLTVVHQDWLNGKEYKCKVSNKGLPAPIEKTISKTKGQP | |
| Fc fragment | REPQVYTLPPSREEMTKNQVSLTCLVKGFYPSDIAVEWESNGQPENNYKTTPPMLD | |
| | SDGSFFLYSKLTVDKSRWQQGNVFSCSVMHEALHNHYTQKSLSLSPG | |
| Human IgG4 | APEFLGGPSVFLFPPKPKDTLMISRTPEVTCVVVDVSQEDPEVQFNWYVDGVEVHN | 678 |
| K4474 | AKTKPREEQFNSTYRVVSVLTVLHQDWLNGKEYKCKVSNKGLPSSIEKTISKAKGQP | |
| Fc fragment | REPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLG | |
| Human IgG1 | ASTKGPSVFPLAPSSKSTSGGTAALGCLVKDYFPEPVTVSWNSGALTSGVHTFPAVL | 679 |
| L235E | QSSGLYSLSSVVTVPSSSLGTQTYICNVNHKPSNTKVDKKVEPKSCDKTHTCPPCPAP | |
| CH1-CH3 | ELeGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Human IgG1 | ASTKGPSVFPLAPSSKSTSGGTAALGCLVKDYFPEPVTVSWNSGALTSGVHTFPAVL | 680 |
| L235A/G237A | QSSGLYSLSSVVTVPSSSLGTQTYICNVNHKPSNTKVDKKVEPKSCDKTHTCPPCPAP | |
| CH1-CH3 | ELaGaPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Human IgG1 | ASTKGPSVFPLAPSSKSTSGGTAALGCLVKDYFPEPVTVSWNSGALTSGVHTFPAVL | 681 |
| L235E/P329G | QSSGLYSLSSVVTVPSSSLGTQTYICNVNHKPSNTKVDKKVEPKSCDKTHTCPPCPAP | |
| CH1-CH3 | ELeGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALgAPIEKTISKAKGQPRE | |
| | PQVYTLPPSRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Human IgG1 | ASTKGPSVFPLAPSSKSTSGGTAALGCLVKDYFPEPVTVSWNSGALTSGVHTFPAVL | 682 |
| N297A | QSSGLYSLSSVVTVPSSSLGTQTYICNVNHKPSNTKVDKKVEPKSCDKTHTCPPCPAP | |
| CH1-CH3 | ELLGGPSVFLFPPKPKDTLMISRTPEVTCVVVDVSHEDPEVKFNWYVDGVEVHNAK | |
| | TKPREEQYaSTYRVVSVLTVLHQDWLNGKEYKCKVSNKALPAPIEKTISKAKGQPREP | |
| | QVYTLPPSRDELTKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| Human IgG1 | ASTKGPSVFPLAPSSKSTSGGTAALGCLVKDYFPEPVTVSWNSGALTSGVHTFPAVL | 683 |
| A330S/P331S | QSSGLYSLSSVVTVPSSSLGTQTYICNVNHKPSNTKVDKKVEPKSCDKTHTCPPCPAP | |
| CH1-CH3 | ELLGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPSSIEKTISKAKGQPREP | |
| | QVYTLPPSRDELTKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| Human IgG1 | ASTKGPSVFPLAPSSKSTSGGTAALGCLVKDYFPEPVTVSWNSGALTSGVHTFPAVL | 684 |
| K4474 | QSSGLYSLSSVVTVPSSSLGTQTYICNVNHKPSNTKVDKKVEPKSCDKTHTCPPCPAP | |
| CH1-CH3 | ELLGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPG | |
| Human IgG2 | ASTKGPSVFPLAPCSRSTSESTAALGCLVKDYFPEPVTVSWNSGALTSGVHTFPAVL | 685 |
| V235E | QSSGLYSLSSVVTVPSSNFGTQTYTCNVDHKPSNTKVDKTVERKCCVECPPCPAPPe | |
| CH1-CH3 | AGPSVFLFPPKPKDTLMISRTPEVTCVVVDVSHEDPEVQFNWYVDGVEVHNAKTKP | |
| | REEQFNSTFRVVSVLTVVHQDWLNGKEYKCKVSNKGLPAPIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPGK | |
| Human IgG2 | ASTKGPSVFPLAPCSRSTSESTAALGCLVKDYFPEPVTVSWNSGALTSGVHTFPAVL | 686 |
| V235A/G237A | QSSGLYSLSSVVTVPSSNFGTQTYTCNVDHKPSNTKVDKTVERKCCVECPPCPAPPa | |
| CH1-CH3 | AaPSVFLFPPKPKDTLMISRTPEVTCVVVDVSHEDPEVQFNWYVDGVEVHNAKTKP | |
| | REEQFNSTFRVVSVLTVVHQDWLNGKEYKCKVSNKGLPAPIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPGK | |
| Human IgG2 | ASTKGPSVFPLAPCSRSTSESTAALGCLVKDYFPEPVTVSWNSGALTSGVHTFPAVL | 687 |
| V235E/P329G | QSSGLYSLSSVVTVPSSNFGTQTYTCNVDHKPSNTKVDKTVERKCCVECPPCPAPPe | |
| CH1-CH3 | AGPSVFLFPPKPKDTLMISRTPEVTCVVVDVSHEDPEVQFNWYVDGVEVHNAKTKP | |
| | REEQFNSTFRVVSVLTVVHQDWLNGKEYKCKVSNKGLgAPIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPGK | |
| Human IgG2 | ASTKGPSVFPLAPCSRSTSESTAALGCLVKDYFPEPVTVSWNSGALTSGVHTFPAVL | 688 |
| N297A | QSSGLYSLSSVVTVPSSNFGTQTYTCNVDHKPSNTKVDKTVERKCCVECPPCPAPPV | |
| CH1-CH3 | AGPSVFLFPPKPKDTLMISRTPEVTCVVVDVSHEDPEVQFNWYVDGVEVHNAKTKP | |
| | REEQFaSTFRVVSVLTVVHQDWLNGKEYKCKVSNKGLPAPIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPGK | |
| Human IgG2 | ASTKGPSVFPLAPCSRSTSESTAALGCLVKDYFPEPVTVSWNSGALTSGVHTFPAVL | 689 |
| A330S/P331S | QSSGLYSLSSVVTVPSSNFGTQTYTCNVDHKPSNTKVDKTVERKCCVECPPCPAPPV | |
| CH1-CH3 | AGPSVFLFPPKPKDTLMISRTPEVTCVVVDVSHEDPEVQFNWYVDGVEVHNAKTKP | |
| | REEQFNSTFRVVSVLTVVHQDWLNGKEYKCKVSNKGLPSSIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPGK | |
| Human IgG2 | ASTKGPSVFPLAPCSRSTSESTAALGCLVKDYFPEPVTVSWNSGALTSGVHTFPAVL | 690 |
| K4474 | QSSGLYSLSSVVTVPSSNFGTQTYTCNVDHKPSNTKVDKTVERKCCVECPPCPAPPV | |
| CH1-CH3 | AGPSVFLFPPKPKDTLMISRTPEVTCVVVDVSHEDPEVQFNWYVDGVEVHNAKTKP | |
| | REEQFNSTFRVVSVLTVVHQDWLNGKEYKCKVSNKGLPAPIEKTISKTKGQPREPQV | |
| | YTLPPSREEMTKNQVSLTCLVKGFYPSDIAVEWESNGQPENNYKTTPPMLDSDGSF | |
| | FLYSKLTVDKSRWQQGNVFSCSVMHEALHNHYTQKSLSLSPG | |
| Human IgG4 | ASTKGPSVFPLAPCSRSTSESTAALGCLVKDYFPEPVTVSWNSGALTSGVHTFPAVL | 691 |
| L235E | QSSGLYSLSSVVTVPSSSLGTKTYTCNVDHKPSNTKVDKRVESKYGPPCPSCPAPEFe | |
| CH1-CH3 | GGPSVFLFPPKPKDTLMISRTPEVTCVVVDVSQEDPEVQFNWYVDGVEVHNAKTK | |
| | PREEQFNSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Human IgG4 | ASTKGPSVFPLAPCSRSTSESTAALGCLVKDYFPEPVTVSWNSGALTSGVHTFPAVL | 692 |
| L235A/G237A | QSSGLYSLSSVVTVPSSSLGTKTYTCNVDHKPSNTKVDKRVESKYGPPCPSCPAPEFa | |
| CH1-CH3 | GaPSVFLFPPKPKDTLMISRTPEVTCVVVDVSQEDPEVQFNWYVDGVEVHNAKTKP | |
| | REEQFNSTYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVLDSDGSFF | |
| | LYSRLTVDKSRWQEGNVFSCSVMHEALHNHYTQKSLSLSLGK | |
| Human IgG4 | ASTKGPSVFPLAPCSRSTSESTAALGCLVKDYFPEPVTVSWNSGALTSGVHTFPAVL | 693 |
| N297A | QSSGLYSLSSVVTVPSSSLGTKTYTCNVDHKPSNTKVDKRVESKYGPPCPSCPAPEFL | |
| CH1-CH3 | GGPSVFLFPPKPKDTLMISRTPEVTCVVVDVSQEDPEVQFNWYVDGVEVHNAKTK | |
| | PREEQFaSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Human IgG4 | ASTKGPSVFPLAPCSRSTSESTAALGCLVKDYFPEPVTVSWNSGALTSGVHTFPAVL | 694 |
| L235E/P329G | QSSGLYSLSSVVTVPSSSLGTKTYTCNVDHKPSNTKVDKRVESKYGPPCPSCPAPEFe | |
| CH1-CH3 | GGPSVFLFPPKPKDTLMISRTPEVTCVVVDVSQEDPEVQFNWYVDGVEVHNAKTK | |
| | PREEQFNSTYRVVSVLTVLHQDWLNGKEYKCKVSNKGLgSSIEKTISKAKGQPREPQ | |
| | VYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Human IgG4 | ASTKGPSVFPLAPCSRSTSESTAALGCLVKDYFPEPVTVSWNSGALTSGVHTFPAVL | 695 |
| K4474 | QSSGLYSLSSVVTVPSSSLGTKTYTCNVDHKPSNTKVDKRVESKYGPPCPSCPAPEFL | |
| CH1-CH3 | GGPSVFLFPPKPKDTLMISRTPEVTCVVVDVSQEDPEVQFNWYVDGVEVHNAKTK | |
| | PREEQFNSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLG | |

TABLE 2
HUMAN LIGHT CHAIN CONSTANT
REGION SEQUENCES

| | | SEQ |
|---|---|---|
| | | ID |
| Sequence name | Sequence | NO |
| Human kappa light | RTVAAPSVFIFPPSDEQLKS | 4 |
| chain constant | GTASVVCLLNNFYPREAKVQ | |
| region | WKVDNALQSGNSQESVTEQD | |
| | SKDSTYSLSSTLTLSKADYE | |
| | KHKVYACEVTHQGLSSPVTK | |
| | SFNRGEC | |
| Human lambda light | GQPKAAPSVTLFPPSSEELQ | 35 |
| chain constant | ANKATLVCLISDFYPGAVTV | |
| region | AWKADSSPVKAGVETTTPSK | |
| | QSNNKYAASSYLSLTPEQWK | |
| | SHRSYSCQVTHEGSTVEKTV | |
| | APTECS | |

### Exemplary Variants

In certain examples the wild type (“WT”) heavy chain constant region comprises an unmodified hIgG1 heavy chain of SEQ ID NO:5. However, as described above, the amino acid variants in Table 3 can be present in a Fc region of various other molecules including IgG2, IgG4, and variants thereof (Table 1).

TABLE 3
FC VARIANTS

| | IgG1 Heavy chain |
|---|---|
| | constant region SEQ |
| Amino acid substitutions in variant | ID NO: |
| M252Y/S254T/T256E/V308P/N434W | 36 |
| M252Y/N286E/V308P/M428I/N434Y | 37 |
| M252Y/N434Y/Y436V | 38 |
| M252Y/V308P/N434W | 39 |
| M252Y/V308P/N434Y | 40 |
| M252Y/T256E/V308P/N434W | 41 |
| M252Y/S254T/T256E/N434W | 42 |
| M252Y/T256E/V308P | 44 |
| M252F/V308P | 46 |
| Wild Type (WT) | 47 |
| M252Y/S254T/T256E/V308W | 48 |
| M252Y/S254T/T256E/V308P | 51 |
| M252Y/V308P | 52 |
| M252Y/S254T/V308P | 53 |
| S254T/T256E/V308P/N434W | 54 |
| M252Y/S254T/V308P/N434W | 55 |
| P257A/V308P/M428L/N434Y | 56 |
| N434G | 57 |
| H435N | 58 |
| M252Y | 59 |
| T256E/V308P | 60 |
| V308N | 61 |
| V308F | 62 |
| M252Y/S254T/T256E | 63 |
| H433T | 64 |
| S254T/T256E/V308P | 65 |
| H435K | 66 |
| M252W | 67 |
| H433E | 68 |
| M252Y/S254T/T256E/V308S | 69 |
| V308P | 70 |
| M252Y/S254T/T256E/V308A | 71 |
| N434F | 72 |
| R255Y | 73 |
| M252Y/S254T/T256E/V308I | 74 |
| H433Q | 75 |
| N434Q | 76 |
| M252Y/S254T/T256E/V308F | 77 |
| S254T/V308P | 78 |
| N434A | 79 |
| N434L | 80 |
| H433L | 81 |
| P257A | 82 |
| V308M | 83 |
| H433P | 84 |
| V308I | 85 |
| H435R | 86 |
| M252Y/S254T/T256E/V308M | 87 |
| N434M | 88 |
| H433N | 89 |
| N434H | 90 |
| N434V | 91 |
| N434I | 92 |
| M252Y/S254T/T256E/V308G | 93 |
| M252F | 94 |
| M252Y/S254T/T256E/V308T | 95 |
| V308G | 96 |
| N434S | 97 |
| H433R | 98 |
| H433Y | 99 |
| M252Y/S254T/T256E/V308L | 100 |
| N434K | 101 |
| V308A | 102 |
| V308E | 103 |
| M252G | 104 |
| N434Y | 105 |
| V308H | 106 |
| H433I | 107 |
| R255G | 108 |
| H433C | 109 |
| V308S | 110 |
| M252L | 111 |
| M252A | 112 |
| M252K | 113 |
| V308T | 114 |
| V308Q | 115 |
| H433D | 116 |
| M252E | 117 |
| N434T | 118 |
| H433A | 119 |
| M252H | 120 |
| N434R | 121 |
| M252R | 122 |
| H433V | 123 |
| N434E | 124 |
| V308L | 125 |
| N434W | 126 |
| M252Y/S254T/T256E/V308R | 127 |
| M252N | 128 |
| M252Y/S254T/T256E/V308Q | 129 |
| M252Q | 130 |
| H435L | 131 |
| N434D | 132 |
| H435D | 133 |
| H435G | 134 |
| N434P | 135 |
| H435E | 136 |
| V308D | 137 |
| M252V | 138 |
| M252Y/S254T/T256E/V308K | 139 |
| H435Q | 140 |
| M252I | 141 |
| M252Y/S254T/T256E/V308D | 142 |
| M252T | 143 |
| M252D | 144 |
| H435I | 145 |
| M252P | 146 |
| V308W | 147 |
| V308R | 148 |
| M252Y/S254T/T256E/V308N | 149 |
| M252Y/S254T/T256E/V308E | 150 |
| M252Y/S254T/T256E/V308H | 151 |
| V308K | 152 |
| V308Y | 153 |
| M252Y/S254T/T256E/V308Y | 154 |
| M252Y/S254R/T256I/V308P/N434Y | 155 |
| M252Y/S254E/T256P/V308W | 156 |
| S254T/T256E/V308P/N434W | 54 |
| M252Y/T256V/V308P/N434W | 157 |
| M252Y/S254T/T256P/V308P/N434Y | 158 |
| M252Y/V308P/N434W | 39 |
| M252Y/S254T/V308P/N434W | 55 |
| M252Y/T256D/V308P/N434W | 159 |
| M252Y/S254T/T256E/V308P/N434W | 36 |
| V308P/N434W | 160 |
| M252Y/S254T/T256E/N434W | 42 |
| M252Y/T256S/V308P/N434W | 161 |
| M252Y/T256L/V308P/N434W | 162 |
| M252Y/S254T/T256L/V308P/N434Y | 163 |
| M252Y/T256V/V308P/N434F | 164 |
| M252Y/T256P/V308W | 165 |
| M252Y/T256Y/V308P/N434W | 166 |
| M252Y/T256H/V308P/N434W | 167 |
| M252Y/T256P/V308P/N434W | 168 |
| M252Y/S254A/T256D/V308P/N434Y | 169 |
| M252Y/S254V/T256P/V308P/N434Y | 170 |
| M252Y/T256K/V308P/N434W | 171 |
| M252Y/S254A/T256V/V308P/N434Y | 172 |
| M252Y/S254I/T256R/V308P/N434Y | 173 |
| M252Y/T256M/V308P/N434W | 174 |
| M252Y/T256G/V308P/N434P | 175 |
| M252Y/T256R/V308P/N434W | 176 |
| M252Y/S254A/T256A/V308P/N434Y | 177 |
| M252Y/T256E/V308P/N434W | 41 |
| M252Y/T256A/V308P/N434W | 178 |
| M252Y/S254G/T256V/V308P/N434Y | 179 |
| M252Y/V308P/N434Y | 40 |
| M252Y/S254A/T256N/V308P/N434Y | 180 |
| M252Y/S254Y/T256Q/V308P/N434Y | 181 |
| M252Y/T256G/V308P/N434W | 182 |
| M252Y/S254R/T256R/V308P/N434Y | 183 |
| M252Y/S254L/T256E/V308P/N434Y | 184 |
| M252Y/T256E/V308P/N434F | 185 |
| M252Y/T256I/V308P/N434W | 186 |
| M252Y/T256W/V308P/N434W | 187 |
| M252Y/T256Y/V308P/N434H | 188 |
| M252Y/S254G/T256E/V308P/N434Y | 189 |
| M252Y/T256I/V308P/N434Y | 190 |
| M252Y/T256R/V308P/N434Y | 191 |
| M252Y/S254V/T256S/V308P/N434Y | 192 |
| M252Y/T256P/V308P/N434F | 193 |
| M252Y/S254G/T256A/V308P/N434Y | 194 |
| M252Y/S254H/V308P/N434Y | 195 |
| M252Y/S254R/T256S/V308P/N434Y | 196 |
| M252Y/S254L/T256V/V308P/N434Y | 197 |
| M252Y/S254I/T256E/V308P/N434Y | 198 |
| M252Y/S254H/T256D/V308P/N434Y | 199 |
| M252Y/T256V/V308P/N434Y | 200 |
| M252Y/S254H/T256V/V308P/N434Y | 201 |
| M252Y/S254L/T256S/V308P/N434Y | 202 |
| M252Y/S254A/T256L/V308P/N434Y | 203 |
| M252Y/T256A/V308P/N434F | 204 |
| M252Y/T256H/V308P/N434P | 205 |
| M252Y/T256K/V308P/N434Y | 206 |
| M252Y/T256E/V308P/N434Y | 207 |
| M252Y/T256N/V308P/N434Y | 208 |
| M252Y/T256G/V308P/N434F | 209 |
| M252Y/S254N/T256G/V308P/N434Y | 210 |
| M252Y/T256W/V308P/N434Y | 211 |
| M252Y/S254G/T256G/V308P/N434Y | 212 |
| M252Y/S254T/T256A/V308P/N434Y | 213 |
| M252Y/S254V/T256D/V308P/N434Y | 214 |
| M252Y/T256I/V308P/N434V | 215 |
| M252Y/S254F/V308P/N434Y | 216 |
| M252Y/T256E/V308P/N434H | 217 |
| M252Y/T256P/V308P/N434Y | 218 |
| M252Y/S254A/T256S/V308P/N434Y | 219 |
| M252Y/T256S/V308P/N434Y | 220 |
| M252Y/S254I/T256V/V308P/N434Y | 221 |
| M252Y/T256S/V308P/N434F | 222 |
| M252Y/T256D/V308P/N434Y | 223 |
| M252Y/T256S/V308P/N434H | 224 |
| M252Y/S254R/T256E/V308P/N434Y | 225 |
| M252Y/S254T/T256M/V308P/N434Y | 226 |
| M252Y/T256Y/V308P/N434Y | 227 |
| M252Y/T256S/V308P/N434G | 228 |
| M252Y/S254A/T256R/V308P/N434Y | 229 |
| M252Y/T256V/V308P/N434G | 230 |
| M252Y/T256H/V308P/N434Y | 231 |
| M252Y/T256V/V308P/N434M | 232 |
| M252Y/S254A/T256P/V308P/N434Y | 233 |
| M252Y/S254Q/T256L/V308P/N434Y | 234 |
| M252Y/V308P | 52 |
| M252Y/T256P/V308P/N434A | 235 |
| M252Y/T256F/V308P/N434W | 236 |
| M252Y/S254G/T256L/V308P/N434Y | 237 |
| M252Y/S254G/T256Q/V308P/N434Y | 238 |
| M252Y/T256G/V308P/N434Y | 239 |
| M252Y/S254G/T256S/V308P/N434Y | 240 |
| N434W | 126 |
| M252Y/T256A/V308P/N434Y | 241 |
| M252Y/T256V/V308P/N434T | 242 |
| M252Y/S254G/T256R/V308P/N434Y | 243 |
| M252Y/S254V/T256I/V308P/N434Y | 244 |
| M252Y/T256R/V308P/N434S | 245 |
| M252Y/S254L/T256R/V308P/N434Y | 246 |
| M252Y/T256L/V308P/N434F | 247 |
| S254W | 248 |
| M252Y/S254G/T256W/V308P/N434Y | 249 |
| M252Y/S254R/T256Q/V308P/N434Y | 250 |
| M252Y/S254L/T256D/V308P/N434Y | 251 |
| M252Y/S254R/T256N/V308P/N434Y | 252 |
| M252Y/S254I/T256I/V308P/N434Y | 253 |
| M252Y/T256F/V308P/N434Y | 254 |
| M252Y/S254A/T256E/V308P/N434Y | 255 |
| M252Y/S254M/T256R/V308P/N434Y | 256 |
| M252Y/S254V/T256N/V308P/N434Y | 257 |
| M252Y/T256D/V308P | 258 |
| M252Y/S254G/T256N/V308P/N434Y | 259 |
| M252Y/T256F/V308P/N434F | 260 |
| M252Y/S254A/T256K/V308P/N434Y | 261 |
| M252Y/T256G/V308P/N434H | 262 |
| S254F | 263 |
| M252Y/S254G/V308P/N434Y | 264 |
| M252Y/T256D/V308P/N434T | 265 |
| M252Y/T256Q/V308P/N434W | 266 |
| M252Y/T256E/V308P/N434A | 267 |
| M252Y/S254L/T256G/V308P/N434Y | 268 |
| M252Y/V308P/N434H | 269 |
| M252Y/T256Q/V308P/N434Y | 270 |
| R255G | 108 |
| M252Y/T256L/V308P/N434Y | 271 |
| M252Y/T256G/V308P/N434R | 272 |
| M252Y/S254E/T256P/V308P/N434Y | 273 |
| M252Y/S254I/T256M/V308P/N434Y | 274 |
| M252Y/T256P/V308P/N434H | 275 |
| M252Y/S254A/V308P/N434Y | 276 |
| M252Y/S254L/T256A/V308P/N434Y | 277 |
| M252Y/S254T/T256Y/V308P/N434Y | 278 |
| M252Y/T256A/V308P/N434S | 279 |
| M252Y/T256H/V308P/N434S | 280 |
| M252Y/S254Q/T256V/V308P/N434Y | 281 |
| M252Y/T256I/V308P/N434T | 282 |
| M252Y/S254T/T256E/V308P | 51 |
| M252Y/S254V/T256K/V308P/N434Y | 283 |
| M252Y/T256G/V308P/N434K | 284 |
| M252Y/T256W/V308P | 285 |
| M252Y/T256R/V308P/N434A | 286 |
| M252Y/T256S/V308P/N434K | 287 |
| M252Y/V308P/N434S | 288 |
| M252Y/T256R/V308P/N434V | 289 |
| M252Y/T256Q/V308P | 290 |
| M252Y/T256G/V308P/N434S | 291 |
| N434Y | 105 |
| M252Y/S254T/V308P | 53 |
| M252Y/T256L/V308P/N434I | 292 |
| M252Y/V308F | 293 |
| M252Y/T256V/V308P/N434R | 294 |
| M252Y/T256S/V308P/N434T | 295 |
| M252Y/T256P/V308P/N434G | 296 |
| M252Y/T256E/V308P/N434G | 297 |
| M252Y/T256V/V308P/N434I | 298 |
| M252Y/T256H/V308P | 299 |
| M252I/V308S | 300 |
| M252Y/T256P/V308P/N434I | 301 |
| M252Y/T256E/V308P/N434S | 302 |
| M252Y/T256N/V308P | 303 |
| N434F | 72 |
| M252Y/T256R/V308P/N434G | 304 |
| M252Y/T256K/V308P/N434G | 305 |
| M252Y/S254T/T256E/V308M | 87 |
| M252Y/T256W/V308P/N434S | 306 |
| M252Y/T256D/V308P/N434S | 307 |
| P257A | 82 |
| M252Y/T256Q/V308P/N434L | 308 |
| M252Y/T256E/V308P/N434R | 309 |
| M252Y/V308P/N434A | 310 |
| M252Y/T256F/V308P/N434S | 311 |
| M252Y/T256K/V308P/N434S | 312 |
| M252Y/V308P/N434R | 313 |
| M252Y/T256E/V308P/N434Q | 314 |
| M252Y/V308P/N434M | 315 |
| M252Y/T256G/V308P/N434M | 316 |
| M252Y/T256W/V308P/N434V | 317 |
| M252Y/T256Y/V308P/N434S | 318 |
| M252Y/T256P/V308P/N434K | 319 |
| R255Y | 73 |
| M252Y/T256S/V308P/N434S | 320 |
| M252Y/T256R/V308P/N434I | 321 |
| M252Y/T256D/V308P/N434P | 322 |
| M252Y/T256Y/V308P/N434V | 323 |
| M252Y/T256L/V308P | 324 |
| M252Y/T256E/V308P | 44 |
| M252Y/V308P/N434P | 325 |
| M252Y/T256L/V308P/N434K | 326 |
| M252Y/T256G/V308P/N434Q | 327 |
| M252Y/T256N/V308P/N434K | 328 |
| M252Y/V308P/N434G | 329 |
| M252Y/T256P/V308P/N434M | 330 |
| M252Y/T256F/V308P/N434R | 331 |
| M252Y/S254T/T256E/V308G | 93 |
| M252Y/V308P/N434Q | 332 |
| M252Y/T256G/V308P | 333 |
| M252Y/T256D/V308P/N434A | 334 |
| M252Y/T256I/V308P/N434I | 335 |
| M252Y/T256E/V308P/N434P | 336 |
| R255E | 337 |
| M252Y/T256Y/V308P/N434R | 338 |
| M252Y/T256I/V308P | 339 |
| M252Y/T256R/V308P/N434K | 340 |
| M252W | 67 |
| M252Y/T256R/V308P | 341 |
| M252Y/T256I/V308P/N434P | 342 |
| M252Y/T256E/V308P/N434I | 343 |
| M252Y/V308K | 344 |
| M252Y/T256Y/V308P | 345 |
| M252Y/T256F/V308P | 346 |
| M252S/V308A | 347 |
| M252Y/T256E/V308P/N434T | 348 |
| M252Y/T256L/V308P/N434S | 349 |
| M252T/V308H | 350 |
| M252Y/T256M/V308P | 351 |
| M252Y/T256S/V308P/N434Q | 352 |
| M252V/V308H | 353 |
| M252Y/T256Q/V308P/N434R | 354 |
| M252Y/V308P/N434T | 355 |
| M252Y/T256N/V308P/N434Q | 356 |
| M252N/V308K | 357 |
| M252W/V308G | 358 |
| M252Y/T256I/V308P/N434D | 359 |
| M252T/V308W | 360 |
| M252Y/V308A | 361 |
| M252Y/V308E | 362 |
| M252Y/S254T/T256E/V308F | 77 |
| M252K/V308K | 363 |
| M252Y/T256K/V308P/N434L | 364 |
| M252F/V308F | 365 |
| M252Y/T256N/V308P/N434L | 366 |
| N434H | 90 |
| M252Y/T256W/V308P/N434M | 367 |
| M252Y/V308L | 368 |
| M252Y/V308P/N434I | 369 |
| M252K | 113 |
| M252R/V308P | 370 |
| M252Y/T256R/V308P/N434L | 371 |
| M252Y/T256Q/V308P/N434K | 372 |
| M252Y/T256I/V308P/N434M | 373 |
| M252W/V308S | 374 |
| M252E/V308W | 375 |
| M252Y/T256I/V308P/N434R | 376 |
| M252H/V308M | 377 |
| T256S | 378 |
| M252W/V308H | 379 |
| P257I | 380 |
| M252Y/T256A/V308P/N434K | 381 |
| M252A/V308M | 382 |
| M252N/V308L | 383 |
| M252Y/T256R/V308P/N434R | 384 |
| M252A/V308F | 385 |
| M252Y/T256Y/V308P/N434T | 386 |
| R255H | 387 |
| M252Y/T256L/V308P/N434L | 388 |
| M252Y/T256A/V308P/N434L | 389 |
| M252Y/S254T/T256E/V308Q | 129 |
| M252Y/T256R/V308P/N434Q | 390 |
| M252I/V308A | 391 |
| M252Y/T256I/V308P/N434L | 392 |
| M252Q/V308W | 393 |
| M252Y/T256F/V308P/N434D | 394 |
| P257S | 395 |
| V308M | 83 |
| M252Y/T256L/V308P/N434T | 396 |
| M252I | 141 |
| M252Y/T256F/V308P/N434L | 397 |
| M252Y/S254T/T256E | 63 |
| M252L/V308W | 398 |
| WT | 47 |
| M252Y/T256L/V308P/N434P | 399 |
| M252Y/S254T/T256E/V308A | 71 |
| M252Y/T256E/V308P/N434K | 400 |
| M252Y/T256F/V308P/N434E | 401 |
| M252Y/T256E/V308P/N434L | 402 |
| V308W | 147 |
| T256D | 403 |
| M252Y/T256A/V308P | 404 |
| M252E/V308L | 406 |
| M252Y/T256A/V308P/N434I | 407 |
| M252F/V308W | 408 |
| M252F/V308H | 409 |
| P257N | 410 |
| M252G/V308S | 411 |
| M252S/V308F | 412 |
| M252Y/V308P/N434D | 413 |
| M252H/V308W | 414 |
| M252Y/T256L/V308P/N434R | 415 |
| M252Y/S254T/T256E/V308R | 127 |
| M252Y/T256A/V308P/N434H | 416 |
| M252Y/T256E/V308P/N434E | 417 |
| M252F/V308D | 418 |
| V308F | 62 |
| M252Y/T256D/V308P/N434E | 419 |
| M252Y/T256G/V308P/N434L | 420 |
| M252R/V308F | 421 |
| M252A/V308W | 422 |
| M252V/V308A | 423 |
| M252Y/T256G/V308P/N434I | 424 |
| M252F/V308K | 425 |
| T256M | 426 |
| M252W/V308R | 427 |
| M252Y/V308P/N434L | 428 |
| M252A/V308R | 429 |
| M252F | 94 |
| M252Q/V308S | 430 |
| M252G | 104 |
| M252P/V308F | 431 |
| M252Y/S254T/T256E/V308K | 139 |
| M252E/V308F | 432 |
| M252H/V308N | 433 |
| M252Y/T256G/V308P/N434D | 434 |
| M252Y/V308I | 435 |
| M252T | 143 |
| M252L | 111 |
| M252Y/T256D/V308P/N434R | 436 |
| M252V | 138 |
| M252A/V308L | 437 |
| M252P | 146 |
| M252Q/V308F | 438 |
| V308A | 102 |
| M252H/V308P | 439 |
| M252Y/T256V/V308P/N434L | 440 |
| M252V/V308S | 441 |
| M252D/V308P | 442 |
| M252Y/T256E | 443 |
| M252F/V308E | 444 |
| M252G/V308L | 445 |
| M252W/V308F | 446 |
| M252P/V308M | 447 |
| M252I/V308Y | 448 |
| T256E/V308P | 60 |
| M252Y/T256R/V308P/N434F | 449 |
| M252V/V308L | 450 |
| M252T/V308S | 451 |
| M252A/V308N | 452 |
| M252N/V308E | 453 |
| M252Y/T256G/V308P/N434V | 454 |
| M252W/V308N | 455 |
| M252Q/V308Y | 456 |
| M252A | 112 |
| M252F/V308I | 457 |
| M252I/V308F | 458 |
| M252N | 128 |
| M252H/V308R | 459 |
| M252Y/T256D/V308P/N434H | 460 |
| M252Y/S254T/T256E/V308H | 151 |
| M252L/V308F | 461 |
| V308L | 125 |
| M252I/V308R | 462 |
| M252E/V308D | 463 |
| M252F/V308T | 464 |
| M252K/V308R | 465 |
| M252V/V308F | 466 |
| M252Y/T256R/V308P/N434D | 467 |
| M252Y/V308P/N434K | 468 |
| S254T/T256E/V308P | 65 |
| M252D | 144 |
| M252G/V308F | 469 |
| M252H | 120 |
| M252Y/S254T/T256E/V308I | 74 |
| M252Q/V308L | 470 |
| M252Y/T256E/V308P/N434D | 471 |
| N434V | 91 |
| M252H/V308F | 472 |
| M252V/V308M | 473 |
| M252E | 117 |
| V308H | 106 |
| M2521/V308N | 474 |
| M252Y/T256D/V308P/N434I | 475 |
| M252I/V308M | 476 |
| M252S/V308E | 477 |
| M252P/V308E | 478 |
| M252Y/T256A/V308P/N434G | 479 |
| V308P | 70 |
| M252Y/T256Q/V308P/N434P | 480 |
| M252Y/T256S/V308P/N434D | 481 |
| M252R/V308L | 482 |
| M252Y/T256P/V308P/N434P | 483 |
| M252K/V308D | 484 |
| M252K/V308N | 485 |
| M252Y/T256W/V308P/N434I | 486 |
| M252E/V308R | 487 |
| M252I/V308T | 488 |
| M252T/V308K | 489 |
| M252Y/T256K/V308P/N434E | 490 |
| T256N | 491 |
| M252S/V308Y | 492 |
| M252A/V308S | 493 |
| M252D/V308S | 494 |
| M252Y/T256F/V308P/N434P | 495 |
| M252V/V308E | 496 |
| M252D/V308H | 497 |
| M252S | 498 |
| M252E/V308N | 499 |
| M252Y/T256S/V308P/N434E | 500 |
| M252P/V308D | 501 |
| V308I | 85 |
| M252L/V308Y | 502 |
| M252R | 122 |
| S254F/V308W | 503 |
| M252H/V308A | 504 |
| M252R/V308A | 505 |
| M252Y/V308Y | 506 |
| M252I/V308D | 507 |
| M252R/V308R | 508 |
| M252D/V308R | 509 |
| M252H/V308D | 510 |
| M252T/V308P | 511 |
| M252E/V308S | 512 |
| M252I/V308H | 513 |
| M252I/V308W | 514 |
| M252L/V308D | 515 |
| T256C | 516 |
| M252Y/V308W | 517 |
| T256Y | 518 |
| M252E/V308Y | 519 |
| M252Y/T256P/V308P/N434L | 520 |
| R255V | 521 |
| M252W/V308D | 522 |
| S254V | 523 |
| M252Y/V308H | 524 |
| V308Y | 153 |
| M252R/V308Y | 525 |
| T256I | 526 |
| M252G/V308Q | 527 |
| T256K | 528 |
| M252T/V308E | 530 |
| M252V/V308R | 531 |
| M252D/V308Y | 532 |
| M252I/V308E | 533 |
| M252T/V308N | 534 |
| M252Y/T256M/V308P/N434E | 535 |
| M252Y/V308P/N434E | 536 |
| N434A | 79 |
| N434L | 80 |
| M252E/V308H | 537 |
| N434P | 135 |
| M252V/V308Y | 538 |
| M252R/V308E | 539 |
| M252R/V308N | 540 |
| M252Q/V308R | 541 |
| M252V/V308D | 542 |
| M252Q/V308K | 543 |
| R255A | 544 |
| M252D/V308F | 545 |
| M252R/V308S | 546 |
| M252Y/T256V/V308P/N434D | 547 |
| T256H | 548 |
| M252L/V308L | 549 |
| M252I/V308I | 550 |
| T256V | 551 |
| T256A | 552 |
| S254L | 553 |
| M252G/V308Y | 554 |
| M252I/V308P | 555 |
| M252Q | 130 |
| M252Y/T256S/V308P/N434P | 556 |
| P257T | 557 |
| R255C | 558 |
| M252F/V308Y | 559 |
| V308T | 114 |
| M252P/V308H | 560 |
| M252D/V308N | 561 |
| M252Y/T256H/V308P/N434A | 562 |
| M252Y/T256F/V308P/N434G | 563 |
| T256L | 564 |
| N434G | 57 |
| M252K/V308L | 566 |
| V308D | 137 |
| N434C | 567 |
| M252H/V308I | 568 |
| M252K/V308Y | 569 |
| M252R/V308I | 570 |
| M252S/V308W | 571 |
| V308K | 152 |
| R255T | 572 |
| S254Y | 573 |
| T256Q | 574 |
| S254M | 575 |
| P257Y | 576 |
| T256E | 577 |
| M252K/V308S | 578 |
| M252Y/T256V/V308P/N434P | 579 |
| V308Q | 115 |
| V308S | 110 |
| M252A/V308Y | 580 |
| M252I/V308G | 581 |
| M252K/V308F | 582 |
| M252Y/T256H/V308P/N434F | 583 |
| M252Y/T256P/V308P/N434R | 584 |
| P257D | 585 |
| V308G | 96 |
| P257H | 586 |
| M252G/V308H | 587 |
| M252W/V308E | 588 |
| M252Y/T256S/V308P/N434A | 589 |
| M252Y/T256S/V308P/N434R | 590 |
| M252P/V308G | 591 |
| M252Y/T256Y/V308P/N434E | 592 |
| S254H | 593 |
| M252Y/S254T/T256E/V308D | 142 |
| R255S | 594 |
| M252E/V308Q | 595 |
| N434S | 97 |
| M252N/V308A | 596 |
| M252V/V308G | 597 |
| V308E | 103 |
| N434M | 88 |
| M252N/V308P | 598 |
| M252S/V308D | 599 |
| M252S/V308K | 600 |
| M252T/V308F | 601 |
| P257M | 602 |
| T256W | 604 |
| M252A/V308E | 605 |
| M252S/V308L | 606 |
| M252S/V308R | 607 |
| S254M/V308W | 608 |
| V308N | 61 |
| P257F | 609 |
| S254K | 610 |
| N434E | 124 |
| M252H/V308G | 611 |
| M252Q/V308N | 612 |
| M252Y | 59 |
| S254T/V308P | 78 |
| M252P/V308S | 613 |
| V308R | 148 |
| R255W | 614 |
| T256G | 615 |
| P257G | 616 |
| N434Q | 76 |
| M252P/V308W | 617 |
| M252Y/T256R/V308P/N434E | 618 |
| R255L | 619 |
| N434D | 132 |
| M252D/V308D | 620 |
| S254I/V308W | 621 |
| M252H/V308E | 622 |
| M252Y/T256S/V308P/N434L | 623 |
| S254P | 624 |
| R255D | 625 |
| R255K | 626 |
| R255Q | 627 |
| M252F/V308R | 628 |
| M252R/V308D | 629 |
| M252V/V308Q | 630 |
| S254L/V308W | 631 |
| P257R | 632 |
| M252N/V308D | 633 |
| M252T/V308Q | 634 |
| V308C | 635 |
| P257W | 636 |
| P257C | 637 |
| P257K | 638 |
| P257E | 639 |
| T256F | 640 |
| N434T | 118 |
| N434R | 121 |
| S254I | 641 |
| M252Q/V308D | 642 |
| M252T/V308T | 643 |
| M252Y/T256G/V308P/N434T | 644 |
| S254Y/V308W | 645 |
| R255M | 646 |
| P257L | 647 |
| M252G/V308D | 648 |
| T256R | 649 |
| P257Q | 650 |
| M252D/V308E | 651 |
| M252I/V308Q | 652 |
| R255F | 653 |
| R255I | 49 |
| R255N | 50 |
| M252Y/S254T/T256E/V308P/N434Y | 6 |
| M252Y/V308P/N434F | 19 |
| M252Y/S254T/T256E/V308P/N434F | 405 |
| M252Y/V308P/N434H | 269 |
| M252Y/S254T/T256E/V308P/N434H | 603 |
| S254T/T256E/V308P/N434Y | 654 |
| S254T/T256E/V308P/N434F | 655 |
| S254T/T256E/V308P/N434H | 656 |
| M252Y/S254T/V308P/N434Y | 700 |
| M252Y/S254T/V308P/N434F | 43 |
| M252Y/S254T/V308P/N434H | 45 |

In some embodiments, the modified Fc region which comprises the transport-enhancing amino acid substitutions M252Y/V308P further comprises an additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W.

In some embodiments, the modified Fc region comprising the transport-enhancing amino acid substitutions is a modified Fc region of IgG1. In some embodiments, the modified Fc region comprising the transport-enhancing amino acid substitutions is a modified Fc region of IgG2. In some embodiments, the modified Fc region comprising the transport-enhancing the amino acid substitutions is a modified Fc region of IgG4.

In some embodiments, the molecule of the present disclosure comprising a modified Fc region which comprises the transport-enhancing amino acid substitutions M252Y/V308P further comprises an additional two transport-enhancing amino acid substitutions in the modified Fc selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256L/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y.

In some embodiments, the modified Fc region comprising the transport-enhancing amino acid substitutions is a modified Fc region of human IgG1. In some embodiments, the modified Fc region comprising the transport-enhancing amino acid substitutions is a modified Fc region of human IgG2. In some embodiments, the modified Fc region comprising the transport-enhancing amino acid substitutions is a modified Fc region of human IgG4.

In particular embodiments, the modified Fc region of the present disclosure comprising the transport-enhancing amino acid substitutions M252Y/V308P further comprises an additional two transport-enhancing amino acid substitutions selected from the group consisting of T256V/N434F; T256E/N434H; T256S/N434W; T256W/N434Y; T256E/N434F; T256R/N434Y; T256P/N434W; T256E/N434Y; T256F/N434F; T256W/N434W; T256F/N434Y; T256I/N434W; T256Q/N434W; T256E/N434W; T256A/N434W; T256E/N434P; T256V/N434W; T256I/N434Y; T256R/N434W; T256G/N434Y; T256I/N434Y; T256V/N434Y; T256Y/N434Y; T256N/N434Y; T256Q/N434Y; T256A/N434Y; T256P/N434Y; T256S/N434Y; T256D/N434W; and T256H/N434F.

In some embodiments, the modified Fc region comprising the transport-enhancing amino acid substitutions is a modified Fc region of IgG1. In some embodiments, the modified Fc region comprising the transport-enhancing amino acid substitutions is a modified Fc region of IgG2. In some embodiments, the modified Fc region comprising the transport-enhancing amino acid substitutions is a modified Fc region of IgG4.

In some embodiments, the modified Fc region of the present disclosure comprising the transport-enhancing amino acid substitutions M252Y/V308P further comprises an additional three transport-enhancing amino substitutions selected from the group consisting of S254T/T256E/N434Y; S245T/T256E/N434F; and S254T/T256E/N434H, with amino acid residue numbering according to EU numbering.

In some embodiments, the modified Fc region comprising the transport-enhancing amino acid substitutions is a modified Fc region of IgG1. In some embodiments, the modified Fc region comprising the transport-enhancing amino acid substitutions is a modified Fc region of IgG2. In some embodiments, the modified Fc region comprising the transport-enhancing amino acid substitutions is a modified Fc region of IgG4.

In another aspect of the present disclosure, the modified Fc region of the present disclosure comprises the transport-enhancing amino acid substitution(s) selected from the group consisting of S254T/T256E/V308P/N434W; S254T/T256E/N434W; N286E/M428I/N434Y; N434W; T256E/N434W; S254T/T256E; and S254T/N434W.

### Additional Alterations to the Fc

In certain embodiments, other amino acid sequence variants of the modified Fcs provided herein are contemplated that are not related to modulation of transport into the central nervous system. For example, it may be desirable to modulate one or more antibody effector functions and/or extend or reduce antibody half-life or modulate other biological properties of the modified Fc variants. Amino acid sequence variants of an Fc may be prepared by introducing appropriate modifications into the nucleotide sequence encoding the Fc, or by peptide synthesis. Such modifications include, for example, deletions from, and/or insertions into and/or substitutions of residues within the amino acid sequences of the Fc that do not modulate transport into the brain and/or are present outside the Fc region. Any combination of deletion, insertion, and substitution can be made to arrive at the final construct, provided that the final construct possesses the desired characteristics, i.e., enhanced transport into the central nervous system.

In some embodiments, the modified Fc region of the present disclosure further comprises an amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G.

The molecule of the present disclosure may have other substitutions, additions and/or deletions or any combination of two or three of these introduced outside of the specified sets of amino acids above, e.g., to influence glyscosylation, to increase serum half-life or, for CH3 domains, to provide for knob in hole heterodimerization of polypeptides that comprise the modified CH3 domain (for example as described in WO 1996/027011, WO 1998/050431 or WO 2016/071377) in the construction of a bispecific antibody. Generally, the knob-in-hole method involves introducing a protuberance (“knob”) at the interface of a first heavy chain constant region and a corresponding cavity (“hole”) in the interface of a second heavy chain constant region, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). Such additional mutations are at a position in the polypeptide that does not have a negative effect on FcRn binding.

The Fc region may possess one or more effector functions with the ability to induce particular biological effects on effector cells, such as monocytes, macrophages, neutrophils, dendritic cells, eosinophils, mast cells, platelets, B cells, large granular lymphocytes, Langerhans' cells, natural killer (NK) cells, and cytotoxic T cells upon Fc receptor binding.

Examples of effector functions include, but are not limited to, Clq binding and complement dependent cytotoxicity (CDC), Fc-receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In certain embodiments, the present disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγR I, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82: 1499-1502 (1985); 5,821,337 (see Bruggemann, M. et al., J. Exp. Med. 166: 1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202: 163 (1996); Cragg, M. S. et al., Blood 101: 1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12): 1759-1769 (2006)).

In some embodiments the Fc region is modified to reduce or substantially eliminate effector functions. Non-limiting examples of antibodies with reduced effector function include those with substitution of one or more of Fc residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Fc modifications to reduce or ablate one or more effector functions include L235E substitution with S228P in IgG4 described in WO 1994/029351, the L235A/G237A (LAGA) effector function ablating substitutions for IgG1 described in WO 1998/006248; the L234A/L235A (LALA) substitutions for IgG1 described in Hezarah et al., (2001) J Virology 75(2):12161-12168; the L234A/L235A/P329G substitutions in an IgG1 described in U.S. Pat. No. 8,969,526; and the aglycosylating substitution N297A for IgG1 described in Bolt et al., (1993) European Journal of Immunology 23(2):403-411

In certain embodiments, it may be desirable to create cysteine engineered Fc variants, in which one or more residues of a modified Fc are substituted with cysteine residues. In particular embodiments, the substituted residues occur at accessible sites of the modified Fc. By substituting those residues with cysteine, reactive thiol groups are thereby positioned at accessible sites of the modified Fc and may be used to conjugate the modified Fc to other moieties, such as drug moieties or linker-drug moieties, to create an Fc conjugate, as described further herein. Cysteine engineered Fcs may be generated as described, e.g., in U.S. Pat. Nos. 7,521,541 and 9,000,130.

### Conjugates/Fusions

The present disclosure also provides conjugate molecules comprising a modified Fc region as described herein conjugated or fused directly or indirectly to one or more therapeutic proteins, cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an Fc conjugate molecule comprises a modified Fc region as described herein fused via a peptide bond to a protein, such as a therapeutic protein. In one embodiment the therapeutic protein is fused to the modified Fc region via a peptide bond or via a peptide linker. In another embodiment, the conjugate comprises a therapeutic protein which is fused to an antibody heavy chain via a peptide bond or via a peptide linker, the antibody in turn comprising the modified Fc region. In another embodiment, the conjugate comprises a therapeutic protein which is fused to an antibody light chain via a peptide bond or via a peptide linker, is the antibody in turn comprising the modified Fc region. As used herein, a “therapeutic protein” refers to a protein that, when expressed, confers a beneficial effect on the cell or tissue or mammal in which it is present. Examples of beneficial effects can be alleviation or amelioration of signs or symptoms of a condition or disease, prevention or inhibition of a condition or disease, or imparting a desired characteristic. Such Fc conjugates may be referred to as Fc fusion proteins. Accordingly, as used herein the term “Fc fusion protein” refers to a protein wherein one or more polypeptides are operably linked to an isolated modified Fc region or a modified Fc in an antibody to thereby impart the blood brain barrier transport properties of the invention described herein and optionally the effector functions and/or pharmacokinetics typically contributed by the Fc region to an antibody to the remainder of the fusion partner. The Fc region is a modified IgG Fc region and comprises the transport-enhancing amino acid substitutions M252Y/V308P. Exemplary therapeutic proteins that may be conjugated to a modified Fc region described herein include TNF-R1, CTLA-4, IL-1R1, alpha-L-iduronidase, iduronate-2-sulphatase, N-sulfatase, N-Sulfoglucosamine sulfohydrolase; alpha-N-acetylglucosaminidase, N-acetyl-galactosamine-6-sulfatase, beta-galactosidase, aryl sulphatase B, hyaluronidase 1; beta-glucuronidase, acid alpha-glucosidase, glucocerebrosidase, alpha-galactosidase A, hexosaminidase A, acid sphingomyelinase, sphingomyelin phosphodiesterase; beta-galactocerebrosidase, beta-galactosidase, beta-glucosidase, arylsulfatase A, acid ceramidase, aspartoacylase, palmitoyl-protein thioesterase 1, hexosaminidase B, beta-hexosaminidase, GM2 ganglioside activator, beta-glucuronidase; heparan-alpha-glucosaminide-N-acetyltransferase; N-acetylglucosamine-6-sulfatase; N-acetylgalactosamine-6-sulfatase; Lysosomal acid lipase/cholesteryl ester hydrolase; Formylglycine-generating enzyme, N-Acetylglucosamine-1-phosphotransferase subunits alpha and/or beta; Cystinosin; LAMP2; Lysosomal integral membrane protein; sialin; NPC intracellular cholesterol transporter 1 and/or 2; Mucolipin 1; Palmitoyl-protein thioesterase 1; Tripeptidyl peptidase 1; battenin; Cysteine string protein; Ceroid-lipofuscinosis neuronal protein 5; Transmembrane ER protein; Major facilitator superfamily domain containing 8; Protein CLN8; Cathepsin D; Cathepsin F; granulin; dysbindin; and tripeptidyl amino peptidase 1. In some embodiments, an extracellular domain of the therapeutic protein is conjugated to a modified Fc provided herein, such as an extracellular domain of TNF-R1, CTLA-4, or IL-1R1.

In some embodiments, the modified Fc region described herein is fused or conjugated to a neurological disorder drug. In some embodiments, the modified Fc region described herein is coupled with a chemotherapeutic agent. In some embodiments, the modified Fc region described herein is coupled with an imaging agent in order to more efficiently visualize transport of the drug or chemotherapeutic agent into the central nervous system.

Covalent conjugation can either be direct or via a linker. In certain embodiments, direct conjugation is by construction of a protein fusion (i.e., by genetic fusion of the two genes encoding the modified Fc and e.g., the neurological disorder drug and expression as a single protein). In certain embodiments, direct conjugation is by formation of a covalent bond between a reactive group on a modified Fc or antibody and a corresponding group or acceptor on the neurological drug. In certain embodiments, direct conjugation is by modification (i.e., genetic modification) of one of the two molecules to be conjugated to include a reactive group (as nonlimiting examples, a sulfhydryl group or a carboxyl group) that forms a covalent attachment to the other molecule to be conjugated under appropriate conditions. As one nonlimiting example, a molecule (i.e., an amino acid) with a desired reactive group (i.e., a cysteine residue) may be introduced into the antibody or modified Fc and a disulfide bond formed with the neurological drug. Methods for covalent conjugation of nucleic acids to proteins are also known in the art (i.e., photocrosslinking, see, e.g., Zatsepin et al. Russ. Chem. Rev. 74: 77-95 (2005))

Non-covalent conjugation can be by any noncovalent attachment means, including hydrophobic bonds, ionic bonds, electrostatic interactions, and the like, as will be readily understood by one of ordinary skill in the art.

Conjugation may also be performed using a variety of linkers. For example, an antibody and a neurological drug or a modified Fc and a neurological drug may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody, modified Fc, or Fc conjugate. See WO94/11026. Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the neurological drug upon delivery to the brain. For example, an acid-labile linker, peptidase-sensitive linker, photolabile linker, dimethyl linker or disulfide-containing linker (Chari et al., Cancer Res. 52: 127-131 (1992); U.S. Pat. No. 5,208,020) may be used.

The present disclosure also includes, but is not limited to, conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S. A).

In some embodiments, a conjugate is an Fc-drug conjugate or an antibody-drug conjugate (ADC) in which a modified Fc region or an antibody comprising a modified Fc region is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12: 1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065. In some embodiments, an Fc conjugate is provided, which comprises a modified Fc herein conjugated to one or more of the forgoing drugs. In another embodiment, a conjugate comprises an antibody or Fc described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, Sapaonaria officinalis inhibitor, gelonin, mitogellin, restrictocin, phenomycin, enomycin, and the tricothecenes.

In another embodiment, a conjugate comprises an antibody or Fc described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc⁹⁹m or I¹²³, or a spin label suitable for nuclear magnetic resonance (NMR) imaging (also known as magnetic resonance imaging or MRI), such as I¹²³, I¹³¹, indium¹¹¹, fluorine¹⁹, carbon¹³, nitrogen¹⁵, oxygen¹⁷, gadolinium, manganese or iron.

In another embodiment a conjugate comprises an antibody which comprises a modified Fc or a modified Fc described herein conjugated to a polynucleotide, such as an antisense oligonucleotide, a ribonucleic acid, a deoxyrobinucleic acid, a splice switching oligonucleotide, an editing nucleotide, a small activating RNA, a mRNA, a tRNA, a siRNA, short hairpin RNA, microRNA, or an aptamer.

### Antigen Binding Domains

As described above, the modified Fc regions of the present disclosure can be part of a molecule which binds to one or more of a variety of antigen binding domains, particularly for antigens present in the central nervous system (CNS). In some embodiments, the molecule comprising a modified IgG Fc of the present disclosure binds to an extracellular antigen or a cell surface antigen found within the CNS or an antigen which is accessible in the CNS but which is not an extracellular antigen or cell surface antigen. “Cell surface antigen” refers to an antigenic structure expressed by a cell and present on the cell surface to allow access to the molecules comprising modified Fc regions as described herein. Nonlimiting examples of cell surface antigens include Tropomyosin receptor kinase B (TrkB), and Tropomyosin receptor kinase C (TrkC), beta-secretase 1 (BACE1), amyloid beta (Abeta), epidermal growth factor receptor (EGFR), human epidermal growth factor receptor 2 (HER2), tau, apolipoprotein E (ApoE), CD20, huntingtin, prion protein (PrP), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL 1 b), caspase 6, triggering receptor expressed on myeloid cells 2 (TREM2), Clq, paired immunoglobin like type 2 receptor alpha (PILRA), CD33, interleukin 6 (IL6), SIGLEC, tumor necrosis factor alpha (TNFa), tumor necrosis factor receptor superfamily member 1 A (TNFR1), tumor necrosis factor receptor superfamily member IB (TNFR2), apolipoprotein J (ApoJ), Tau protein (e.g., a human Tau protein) or a fragment thereof, phosphorylated Tau protein, an unphosphorylated Tau protein, a splice isoform of Tau protein, an N-terminal truncated Tau protein, a C-terminal truncated Tau protein, and/or a fragment thereof, as well as alpha-synuclein protein (e.g., a human alpha-synuclein protein) or a fragment thereof. A non-limiting example of an antigen found within the CNS which is not an extracellular antigen or a cell surface antigen is leucine rich repeat kinase 2 (LRRK2). In some embodiments, the Fab fragment may bind to a monomeric alpha-synuclein, oligomeric alpha-synuclein, alpha-synuclein fibrils, soluble alpha-synuclein, and/or a fragment thereof.

Molecules with modified Fc regions as described herein which bind to any of the above antigens can be useful in the treatment of various disorders of the central nervous system as described in more detail below.

### Nucleic Acids

Another aspect of the disclosure relates to recombinant nucleic acids including a nucleic acid sequence that encodes molecule comprising a modified Fc region of the disclosure. In some embodiments, the recombinant nucleic acids of the disclosure can be configured as expression cassettes or vectors containing these nucleic acid molecules operably linked to heterologous nucleic acid sequences such as, for example, regulatory sequences which allow in vivo expression of the antibody in a host cell.

Nucleic acid molecules of the present disclosure can be of any length, including for example, between about 1 Kb and about 50 Kb, e.g., between about 1.2 Kb and about 10 Kb, between about 2 Kb and about 15 Kb, between about 5 Kb and about 20 Kb, between about 10 Kb and about 20 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

Accordingly, in some embodiments, provided herein is a nucleic acid molecule including a nucleotide sequence encoding a molecule of the disclosure. In some embodiments, the nucleotide sequence is incorporated into an expression cassette or an expression vector. It will be understood by the skilled artisan that an expression cassette generally includes a construct of genetic material that contains coding sequences of the antibody or antigen-binding fragment thereof and enough regulatory information to direct proper transcription and/or translation of the coding sequences in a recipient cell, in vivo and/or ex vivo. Generally, the expression cassette can be inserted into a vector for targeting to a desired host cell and/or into an individual. As such, in some embodiments, an expression cassette of the disclosure include a coding sequence for a molecule of the disclosure, which is operably linked to expression control elements, such as a promoter, and optionally, any or a combination of other nucleic acid sequences that affect the transcription or translation of the coding sequence.

An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, e.g., operably linked.

In some embodiments, the nucleic acid molecule of the disclosure is incorporated into an expression vector. It will be understood by one skilled in the art that the term “vector” generally refers to a recombinant polynucleotide construct designed for transfer between host cells, and that can be used for the purpose of transformation, e.g., the introduction of heterologous DNA into a host cell. As such, in some embodiments, the vector can be a replicon, such as a plasmid, phage, or cosmid, into which another DNA segment can be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector can refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

The nucleic acid sequences encoding the molecules disclosed herein can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the molecules disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Also provided herein are vectors, plasmids, or viruses containing one or more of the nucleic acid molecules encoding any molecule as disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning. A Laboratory Manual (4th ed.). Cold Spring Harbor, NY: Cold Spring Harbor Laboratory and Sambrook, J., & Russel, D. W. (2001). Molecular Cloning. A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into cells, e.g., eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

For example, a molecule as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, it matters only that the components are compatible with one another. Artisans or ordinary skill are able to make such a determination. Furthermore, if guidance is required in selecting an expression system, skilled artisans can consult P. Jones, “Vectors: Cloning Applications”, John Wiley and Sons, New York, N.Y., 2009.

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of an antibody) can also be included. Those of ordinary skill in the art of molecular biology are familiar with routine procedures for isolating nucleic acid molecules. In the event the nucleic acid molecule is a ribonucleic acid (RNA), molecules can be produced, for example, by in vitro transcription.

### Recombinant Cells and Cell Cultures

The nucleic acid of the present disclosure can be introduced into a host cell, such as, for example, a Chinese hamster ovary (CHO) cell, to produce an engineered or recombinant cell containing the nucleic acid molecule. Introduction of the nucleic acid molecules (e.g., DNA or RNA, including mRNA) or vectors of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery. For example, methods for introduction of heterologous nucleic acid molecules into mammalian cells are known in the art and include dextran-mediated transfection, calcium phosphate precipitation, polybrene-mediated transfection, protoplast fusion, electroporation, encapsulation of the nucleic acid molecule(s) in liposomes, lipid nanoparticle technology, biolistic injection and direct microinjection of the DNA into nuclei. In addition, nucleic acid molecules can be introduced into mammalian cells by viral vectors such as lentivirus or adeno-associated virus. As discussed in greater detail below, in some embodiments, molecule of the present disclosure can be introduced to a subject in nucleic acid form (e.g, DNA or RNA, including mRNA), such that the subject's own cells produce the molecule. The present disclosure further provides modifications to nucleotide sequences encoding the molecules described herein that result in increased expression, increased stability, increased nucleic acid (e.g., mRNA) stability, or improved affinity or specificity of the molecules for cell surface antigens of the CNS.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle or can be delivered by viral or non-viral delivery means and methods known in the art, such as electroporation. For example, introduction of nucleic acids into cells can be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. The molecules of the present disclosure may be prepared and purified using known methods. For example, cDNA sequences encoding a HC (for example the amino acid sequence encoding the Fc region given by SEQ ID NO.52 can be cloned in frame with various variable regions and a LC (for example, the amino acid sequence given by SEQ ID NO.7) into an expression vector, using known methods. The engineered immunoglobulin expression vector may then be stably transfected into engineered cells.

In some embodiments, the engineered cell is a eukaryotic cell. In some embodiments, the engineered cell is an animal cell. In some embodiments, the animal cell is a vertebrate animal cell or an invertebrate animal cell. In some embodiments, the animal cell is a mammalian cell. In some embodiments, the animal cell is a human cell. In some embodiments, the animal cell is a non-human animal cell. In some embodiments, the engineered cell is a non-human primate cell. In some embodiments, the engineered cell is selected from the group consisting of a baby hamster kidney (BHK) cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HEK-293 cell, a human HeLa cell, a human HepG2 cell, a human HUH-7 cell, a human MRC-5 cell, a human muscle cell, a mouse 3T3 cell, a mouse connective tissue cell, a mouse muscle cell, and a rabbit kidney cell. In some embodiments, the engineered cell is a Pichia pastoris cell or a Saccharomyces cerevisiae cell, all of which are also suitable for production of the antibodies that are described in the present invention.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

In another aspect, provided herein are methods for producing a modified IgG Fc region or an IgG antibody comprising a modified Fc region, wherein the methods include growing a recombinant cell as disclosed herein under conditions such that the antibody or modified Fc region is produced.

In some embodiments, the methods for producing a modified IgG Fc region or an IgG antibody comprising a modified Fc region as described herein further include isolating the produced antibody or modified Fc region from the recombinant cell and/or the medium in which the recombinant cell is cultured. Accordingly, the antibodies comprising a modified Fc region or modified Fc region produced by the methods disclosed herein are also within the scope of the disclosure.

### Pharmaceutical Compositions

The molecules of the disclosure can be incorporated into compositions, including pharmaceutical compositions.

In another aspect, the molecules of the disclosure can be incorporated into compositions suitable for various downstream applications, for example, pharmaceutical compositions. Exemplary compositions of the disclosure include pharmaceutical compositions which generally comprise one or more of the antibodies or modified Fc regions, nucleic acids, and a pharmaceutically acceptable excipient, e.g., carrier. In some embodiments, the composition is a sterile composition. In some embodiments, the composition is formulated as a vaccine. In some embodiments, the composition further includes an adjuvant.

The pharmaceutical compositions provided herein can be in any form that allows for the composition to be administered to an individual. In some specific embodiments, the pharmaceutical compositions are suitable for human administration. The scope of the present disclosure includes desiccated, e.g., freeze-dried, compositions comprising a molecule comprising a modified Fc region as described herein, or a pharmaceutical composition thereof that includes a pharmaceutically acceptable carrier but substantially lacks water. As used herein, the term “pharmaceutically acceptable” means approved by a regulatory agency of the Federal or a state government or listed in the U.S. Pharmacopeia or other generally recognized pharmacopeiae for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include, glucose, lactose, sucrose, sodium chloride, propylene glycol, water, and the like. Examples of suitable pharmaceutical carriers are described in “Remington's Pharmaceutical Sciences” by E. W. Martin. In some embodiments, the pharmaceutical composition is sterilely formulated for administration into an individual or an animal (some non-limiting examples include a human, or a mammal). In some embodiments, the individual is a human.

In some embodiments, the pharmaceutical compositions of the present disclosure are formulated to be suitable for the intended route of administration to an individual. For example, the pharmaceutical composition can be formulated to be suitable for parenteral, intraperitoneal, colorectal, intraperitoneal, and intratumoral administration. In some embodiments, the pharmaceutical composition can be formulated for transmucosal, parenteral; intramuscular, subcutaneous, intradermal, intramedullary, intrathecal, direct intraventricular, intravenous, intraperitoneal, intranasal, intraocular, or intra-arterial administration. One of ordinary skilled in the art will appreciate that the formulation should suit the mode of administration.

Formulation of a molecule of the present disclosure to be administered will vary according to the route of administration and formulation selected. An appropriate pharmaceutical composition comprising a molecule of the present disclosure to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The molecules of this disclosure can be lyophilized for storage and reconstituted in a suitable carrier prior to use according to art-known lyophilization and reconstitution techniques.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

### Methods of Production

Modified Fcs, molecules comprising modified Fc region such as antibodies, and Fc fusions may be produced using recombinant methods and compositions known in the art. See, e.g., U.S. Pat. No. 4,816,567. In one embodiment, isolated nucleic acid encoding an antibody, modified Fc, or Fc fusion described herein is provided. In the case of antibodies, such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In some embodiments for expressing antibodies, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In various embodiments, a host cell is eukaryotic, e.g. a Chinese Hamster Ovary (CHO) cell or lymphoid cell (e.g., Y0, NS0, Sp20 cell). In some embodiments, a method of making an antibody, modified Fc, or Fc fusion is provided, wherein the method comprises culturing a host cell comprising a nucleic acid encoding the antibody, modified Fc, or Fc fusion, as provided above, under conditions suitable for expression of the antibody, modified Fc, or Fc fusion, and optionally recovering the antibody, modified Fc, or Fc fusion from the host cell (or host cell culture medium).

For recombinant production of an antibody, modified Fc, or Fc fusion, nucleic acid encoding an antibody, modified Fc, or Fc fusion, e.g., as described above, is isolated and inserted into one or more vectors for further cloning and/or expression in a host cell. For antibodies, such nucleic acid may be readily isolated and sequenced using conventional procedures (e.g., by using oligonucleotide probes that are capable of binding specifically to genes encoding the heavy and light chains of the antibody).

Suitable host cells for cloning or expression of protein-encoding vectors include prokaryotic or eukaryotic cells described herein. For example, Fc-containing proteins may be produced in bacteria, in particular when glycosylation and Fc effector function are not needed. For expression of polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the protein may be isolated from the bacterial cell paste in a soluble fraction and can be further purified.

In addition to prokaryotes, eukaryotic microbes such as filamentous fungi or yeast are suitable cloning or expression hosts for protein-encoding vectors, including fungi and yeast strains whose glycosylation pathways have been “humanized,” resulting in the production of a protein with a partially or fully human glycosylation pattern. See Gerngross, Nat. Biotech. 22: 1409-1414 (2004), and Li et al., Nat. Biotech. 24:210-215 (2006).

Suitable host cells for the expression of glycosylated proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are the human fibrosarcoma cell line HT1080, monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3 A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). D. Assays.

### Methods of Delivering a Molecule to the Central Nervous System

In one embodiment the present disclosure provides a method of enhancing the delivery of a molecule comprising an IgG Fc region into the central nervous system, the method comprising modifying the IgG Fc region so that the Fc region comprises the transport-enhancing amino acid substitutions M252Y/V308P and administering the molecule.

In some embodiments, in addition to the transport-enhancing amino acid substitutions M252Y/V308P, the IgG Fc region is modified so that the Fc region further comprises an additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G.

In some embodiments, the IgG Fc region is modified so that the Fc region further comprises, in addition to the transport-enhancing amino acid substitutions M252Y/V308P, an additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W. Thus, in addition to comprising the M252Y/V308P amino acid substitutions, the modified Fc region comprises one additional transport-enhancing amino acid substitutions selected from the above list. In some embodiments, the modified IgG Fc region further comprises an additional two transport-enhancing amino substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256I/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y. In some embodiments, the modified Fc region of the present disclosure comprising the transport-enhancing amino acid substitutions M252Y/V308P further comprises an additional three transport-enhancing amino substitutions selected from the group consisting of S254T/T256E/N434Y; S245T/T256E/N434F; and S254T/T256E/N434H, with amino acid residue numbering according to EU numbering.

Thus, in these embodiments in addition to comprising the M252Y/V308P amino acid substitutions, the modified Fc region comprises two or three additional transport-enhancing amino acid substitutions selected from the above list.

In another embodiment the present disclosure provides a method of treating a disorder associated with the central nervous system, comprising administering a molecule comprising a modified Fc region which enhances transport of the molecule into the central nervous system, wherein the modified Fc region comprises the transport-enhancing amino acid substitutions M252Y/V308P with numbering according to EU.

In some embodiments, the IgG Fc region is modified so that the Fc region further comprises an additional amino acid substitution selected from the group consisting of Y300Q, Y300T, Y300S, Y300K, Y300N, Y300P, Y300D, Y300G, Y300H R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G

In some embodiments, the modified Fc region further comprises, in addition to the transport-enhancing amino acid substitutions M252Y/V308P, an additional transport-enhancing amino acid substitution selected from the group consisting of S254T, T256D, T256E, T256H, T256L, T256N, T256P, T256Q, T256W, N434A, N434G, N434H, N434M, N434P, N434Q, N434R, N434S, and N434W. Thus, in addition to comprising the M252Y/V308P amino acid substitutions, the modified Fc region comprises one additional transport-enhancing amino acid substitutions selected from the above list. In some embodiments, the modified IgG Fc region further comprises an additional two transport-enhancing amino substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256L/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y. In some embodiments, the modified Fc region of the present disclosure comprising the transport-enhancing amino acid substitutions M252Y/V308P further comprises an additional three transport-enhancing amino substitutions selected from the group consisting of S254T/T256E/N434Y; S245T/T256E/N434F; and S254T/T256E/N434H, with amino acid residue numbering according to EU numbering. Thus, in these embodiments in addition to comprising the M252Y/V308P amino acid substitutions, the modified Fc region comprises two or three additional transport-enhancing amino acid substitutions selected from the above list.

In some embodiments of the methods described herein, the modified Fc is part of an antibody. In some embodiments, the modified Fc is a component of a fusion protein.

As used herein, a disorder which is associated with the central nervous system refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, autism spectrum disorders and a lysosomal storage disease. For the purposes of this disclosure, the CNS will be understood to include the eye, which is normally sequestered from the rest of the body by the blood-retina barrier. Specific examples of neurological disorders include, but are not limited to, Sly syndrome and Sanfilippo syndrome, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS, including brain metastases resulting from cancer elsewhere in the body), stroke, dementia, muscular dystrophy (MD), multiple sclerosis (MS), amyotrophic lateral sclerosis (ALS), cystic fibrosis, Angelman's syndrome, Liddle syndrome, Paget's disease, and traumatic brain injury.

By way of example, the molecules comprising modified Fc regions of the present disclosure can be antibodies which bind to cell surface antigens and/or extracellular antigens and/or secreted antigens within the CNS for the purposes of treatment of disease. Exemplary combinations are shown in Table 4.

TABLE 4
Non-limiting examples of neurological disorder drugs
and the disorders they may be used to treat

| Drug | Neurological disorder |
|---|---|
| Anti-BACE1 Antibody | Alzheimer's, acute and |
| | chronic brain injury, |
| | stroke |
| Anti-Abeta Antibody | Alzheimer's disease |
| Anti-Tau Antibody | Alzheimer's disease, |
| | tauopathies |
| Neurotrophin | Stroke, acute brain |
| | injury, spinal |
| | cord injury |
| Brain-derived neurotrophic factor (BDNF), | Chronic brain injury |
| Fibroblast growth factor 2 (FGF-2) | (Neurogenesis) |
| Anti-Epidermal Growth Factor Receptor | Brain cancer |
| (EGFR)-antibody | |
| Glial cell-line derived neural factor (GDNF) | Parkinson's disease |
| Brain-derived neurotrophic factor (BDNF) | Amyotrophic lateral |
| | sclerosis, depression |
| Anti-sialic acid-binding immunoglobulin-type | Alzheimer's disease |
| lectins (SIGLECs) | |
| Lysosomal enzyme | Lysosomal storage |
| | disorders |
| | of the brain |
| Ciliary neurotrophic factor (CNTF) | Amyotrophic lateral |
| | sclerosis |
| Anti-tropomyosin receptor kinase B (TrkB) | Rett syndrome |
| Anti-pituitary adenylate cyclase-activating | Migraine |
| polypeptide (PACAP) | |
| Anti-leucine-rich repeat and immunoglobulin- | Multiple sclerosis |
| like domain containing NOGO receptor- | |
| interacting protein 1 (LINGO-1) | |

Molecules and Fc conjugates/fusions of the disclosure are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The molecule or Fc conjugate/fusion need not be, but is optionally formulated with one or more agents currently used to prevent or treat the disorder in question or to prevent, mitigate or ameliorate one or more side effects of molecule or Fc conjugate administration. The effective amount of such other agents depends on the amount of the molecule or Fc conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a molecule or Fc conjugate of the present disclosure (when used alone or in combination with one or more other additional therapeutic agents) will depend on the type of disease to be treated, the type of molecule or Fc conjugate, the severity and course of the disease, whether the molecule or Fc conjugate is administered for preventive or therapeutic purposes, previous therapy, the patient's clinical history and response to the molecule or Fc conjugate, and the discretion of the attending physician. The molecule or Fc conjugate is suitably administered to the patient at one time or over a series of treatments. Depending on the type and severity of the disease, from about 1 pg/kg to about 15 mg/kg, for example from about 0.1 mg/kg to about 10 mg/kg, of the molecule or Fc conjugate can be an initial candidate dosage for administration to the patient, whether, for example, by one or more separate administrations, or by continuous infusion. One typical daily dosage might range from about 1 pg/kg to 100 mg/kg or more, depending on the factors mentioned above. For repeated administrations over several days or longer, depending on the condition, the treatment would generally be sustained until a desired suppression of disease symptoms occurs. One exemplary dosage of the molecule or Fc conjugate would be in the range from about 0.05 mg/kg to about 40 mg/kg. Thus, one or more doses of about 0.5 mg/kg, 2.0 mg/kg, 4.0 mg/kg, 5.0 mg/kg, 7.5 mg/kg, 10 mg/kg, 15 mg/kg, 20 mg/kg, 25 mg/kg, 30 mg/kg, 35 mg/kg or 40 mg/kg (or any combination thereof) may be administered to the patient. Such doses may be administered intermittently, e.g., every week or every three weeks (e.g., such that the patient receives from about two to about twenty, or e.g., about six doses of the molecule or Fc conjugate). An initial higher loading dose, followed by one or more lower doses may be administered. However, other dosage regimens may be useful. The progress of this therapy is easily monitored by conventional techniques and assays as described herein and as known in the art.

### Systems and Kits

Also provided herein are systems and kits including the molecules comprising modified Fc regions, recombinant nucleic acids, recombinant cells, or pharmaceutical compositions provided and described herein as well as written instructions for making and using the same. For example, provided herein, in some embodiments, are systems and/or kits that include one or more of: molecules comprising modified Fc regions as described herein, a recombinant nucleic acid as described herein, a recombinant cell as described herein, or a pharmaceutical composition as described herein. In some embodiments, the systems and/or kits of the disclosure further include one or more syringes (including pre-filled syringes) and/or catheters used to administer one any of the provided molecules comprising modified Fc regions, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the bispecific binding agents or engineered transmembrane protein.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address or a QR code or bar code encoding a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above. Additional embodiments are disclosed in further detail in the following examples, which are provided by way of illustration and are not in any way intended to limit the scope of this disclosure or the claims.

### Example 1: General Methods

A. Cloning and Purification of Antibody Variants. Antibody variants were cloned using standard molecular biology techniques. Certain variants bearing point mutations at specific residues were engineered according to Kunkel (Proc. Natl. Acad. Sci. USA 82:488 (1985)) using synthetic oligonucleotides that incorporated the targeted point mutation into their sequences. Certain other variants comprising randomly generated mutations were engineered by incorporating random trimer codons into the synthetic oligonucleotides used to amplify the targeted locus by polymerase chain reaction (PCR) and cloning the amplicon into the parent vector by Gibson assembly (Gibson, et al., Nat Methods. 6:343 (2009)). To clone variants comprising multiple mutations spanning longer sections of DNA, BamHI and NheI restriction sites were engineered into the parent plasmid on either side of the locus corresponding to the Fc region, synthesizing double-stranded gene fragments coding the Fc region, and incorporating the desired mutation into the synthetic gene fragment. The gene fragment was then cloned into the parent plasmid by restriction digest and ligation at the BamHI and NheI consensus sequences (Ausubel, F M. Current Protocols in Molecular Biology. (1988)).

Antibody proteins were expressed from the engineered constructs by transfection using the Expi293 expression system (Thermofisher, CA). The culture supernatants containing expressed antibody were filtered through 0.2 μm filters and antibody yield was measured using Protein A sensors on an Octet system (ForteBio).

Antibody variants were purified by affinity chromatography using a Protein A Sepharose resin into 100 mM arginine, 10 mM histidine, 150 mM NaCl, 20 mM Na₂HPO₄, pH 6 buffer, and the final product was concentrated through 30 kDa centrifugal Amicon filters (Millipore) to greater than 5 mg/mL.

B. Antibody Internalization Assay. If transcytosis is a component of the transport of antibodies across the blood brain barrier, then antibody internalization is expected to be detectable in the endothelial cell layer of the blood brain barrier. This assay was developed to identify antibody variants with enhanced endothelial cell internalization properties in a cell line expressing human FcRn (FCGRT; Fc Fragment of IgG Receptor and Transporter; UniProtKB-P55899 (FCGRN_HUMAN); J. Exp. Med. 180:2377-2381(1994)).

Cells of the JEG3 human endothelial placental choriocarcinoma cell line (ECACC 92120308; Kohler and Bridson, J. Clin. Endocrinol. Metab. 32:683 (1971)) were stably induced with a human FCGRT gene (SEQ ID: 21) expression construct and selected using a puromycin resistance selection marker to create JEG3-hFcRn cells. This cell line was chosen because the line is derived from human endothelial cells, which are a major contributor to the blood-brain-barrier. Human FcRn was overexpressed in these cells in order to increase the signal-to-noise for detection of internalization.

The following methods were adapted from the internalization assay described by Corrodus N L et al., J Vis Exp. 2014 Feb. 12; (84). JEG3-hFcRn cells were plated at 100,000 cells/well in a 96 well plate and grown for 1-2 days in Eagle's Modified Essential Media (EMEM), 10% FBS, penicillin/streptomycin, and 10 μg/ml puromycin. JEG3-hFcRn cells were washed with Hanks Buffered Saline Solution (HBSS) leaving 50 μl HBSS in the well.

In some assays, sterile filtered supernatant of transfected Expi293F cells was used for the antibody internalization assay. In these cases, 50 μl sterile filtered supernatant was added directly to the cells in each well. In other assays, antibodies produced by transfected Expi293F cells were first purified prior to use on the antibody internalization assay. In these cases, the purified antibody was diluted in HBSS as a 2× stock, and 50 μl were added to each well to a final concentration of 10 μg/ml.

Cells were incubated with antibody for 3 h at 37° C. and then washed with HBSS, leaving a final volume of 50 μl. Washed cells were fixed by incubating in 4% paraformaldehyde for 5 min at room temperature. Fixed cells were washed again with phosphate buffered saline (PBS), leaving a final volume of 50 μl. Non-specifically bound external antibody was blocked with excess unlabeled secondary anti-human Fab fragment (goat anti-human IgG Fab) diluted in Antibody Blocking Buffer (150 mM NaCl, 50 mM Tris, 1% Bovine Serum Albumin (BSA), 100 mM L-lysine, 0.04% sodium azide, pH 7.4) overnight at room temperature. Cells were then washed with PBS, post fixed in 4% paraformaldehyde for 5 min at room temperature, and washed again with PBS leaving a final volume of cells in PBS of 50 μl. Fixed cells were permeabilized by incubating for 30 min in 50 μl 0.2% Triton X-100, diluted in Antibody Blocking Buffer and then washed with PBS, leaving a final volume of cells in PBS of 50 μl. Fifty μl of 2×DAPI (2 μg/ml) and anti-human-AlexaFluor-647 (Invitrogen, 1:500) diluted in Antibody Blocking Buffer/0.2% TX-100 was added and incubated for 2 h, labeling nuclei and internalized antibodies only.

Following a final PBS wash, stained cells were imaged at 20× using a GE InCell Analyzer. Using the InCell Developer toolbox software, internalized antibody was quantified by creating a threshold above background staining as determined by secondary antibody alone to create a mask. The product of the (density×area) under the mask was then divided by the number of nuclei, which gave the staining intensity of internalized antibody/cell. Each well was visually examined, and wells treated with poorly behaving antibodies, such as those presenting as aggregates or precipitates in the images, were manually excluded from analysis.

C. In Vivo Administration in Mice. Adult male or female Tg276 mice (FcRn−/−hFcRn, JAX #004919) were used for this analysis. These lines carry a knock-out mutation for the mouse Fcgrt (Fc receptor, IgG, alpha chain transporter) gene and a knock in transgene expressing the human FCGRT gene under the control of a widely expressed CAG promoter. Thus, they serve as a model of how FcRn regulates antibody metabolism in humans.

Animals were allowed to acclimate to the test facility for at least 2 days prior to dosing with antibody. Animal body weights were recorded prior to dose administration. Test antibodies targeting the oligodendrocyte marker O4 were administered intravenously at 30 mg/kg. All animals were observed at dosing and at scheduled collection 48 hours post-dosing, and any abnormalities were recorded.

Following inhalation anesthesia at 48 hours post-dosing, blood samples were collected via cardiac venipuncture and the blood collection sites were documented. Blood samples were stored at ambient conditions for at least 30 minutes to allow for clotting and processed to serum by centrifugation (3500 rpm at 5° C. for 10 minutes) within 1 hour of collection. Blood serum samples were stored at nominal −80° C. until further processing.

In some studies CSF samples were also collected immediately following the collection of the blood sample. Individual CSF weights were calculated, the tubes were placed immediately on dry ice, and then stored at −80° C. until further analysis.

D. Immunostaining for Human IgG Kappa Light Chain in Mouse Brains. Following blood and CSF collection, the animals were transcardially perfused with 60 mL of saline followed by 60 mL of 4% w/v paraformaldehyde (PFA). These perfusion steps removed test materials from the luminal compartment of brain blood vessels, including the capillaries, and allowed quantification of the amount of test material found in the brain parenchyma. Brain samples were dissected out from each animal after the animal has been fully perfused. Whole brains were then post-fixed in a 4% w/v PFA solution for 24 hours, after which they were transferred into phosphate buffered saline (PBS) for storage.

Brains were cryoprotected overnight in 20% glycerol and 2% dimethylsulfoxide, and then freeze-sectioned at 35 μm thickness in the coronal plane. Staining was performed through the entire mouse brain on every 12^{th} section (spaced at approximately 420 μm intervals). Brain sections were treated with hydrogen peroxide to inactivate endogenous peroxidases, after which sections were labelled with a biotinylated anti-Human IgG Kappa light chain antibody (Southern Biotech, #2061-08) diluted at 1:500 overnight at room temperature. All incubation solutions from this point onward used Tris buffered saline (TBS; 25 mM Tris-Cl, 130 mM NaCl, 2.7 mM KCl, pH 7.4) and 0.05% Triton X-100 (Promega, #H5141). Following rinses, brain sections were complexed to an avidin-biotin-HRP complex (Vector Laboratories, CA, #PK-6100). The sections were rinsed and then stained with the chromogen, 3,3′-Diaminobenzidine Tetrahydrochloride (DAB), and hydrogen peroxide to create a visible reaction product. Following further rinses, the sections were mounted on gelatin coated glass slides and air-dried. Slides were then counterstained with Thionine-Nissl to reveal cell bodies.

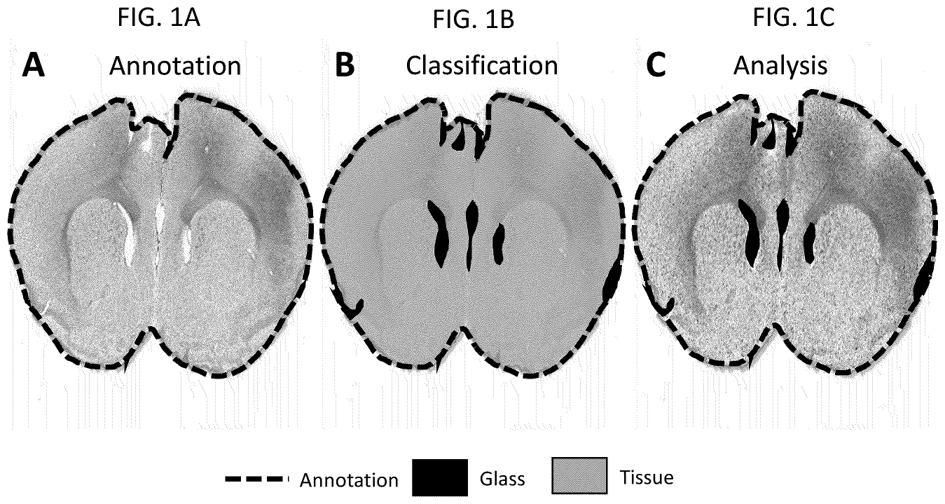

Slides with DAB/Thionine stained brain sections were imaged at 20×. Analysis of the resulting TIFF images was performed on HALO (v3.2, Indica Labs, Albuquerque, NM, USA). For the image analysis, a minimum of 2 rostral to caudal coronal brain sections were selected from each animal. To ensure approximately the same brain regions were compared between different animals, images of sections were also matched based on gross anatomical landmarks. Images of sections from 1-3 animals were analyzed for each test antibody. Using HALO, an annotation contour was drawn along the margins of each coronal brain section in order to include all brain regions in the IHC staining analysis of the section. This approach was selected as it was established empirically that whole-section analysis resulted in lower inter- and intra-animal coefficient of variance, as opposed to restricting the analysis to individual brain regions (e.g. cortex, striatum, etc). The HALO tissue classifier module was applied to the images, which allowed separation of the tissue in the image from non-tissue classes (i.e. glass) based on artificial intelligence random forest algorithm (Breiman L. (2001) Random Forests. Mach Learn. 45: 5-32), trained during a learning session on several representative images. The resulting Tissue-Glass Classifier parameters were then applied to all subsequent images analyzed. The Area Quantification module of HALO was used to quantify the intensity of the DAB staining within the tissue portion of the selected brain section images. Similar to the Tissue-Glass Classifier, parameters for the Area Quantification module were established over a training session on a few representative images in order to teach the algorithm to identify and separate the DAB stain from the Thionine counterstain. During the training session, parameters were established categorizing the DAB-only IHC staining signal as ‘weak,’ ‘moderate,’ or ‘strong’ based on pixel density. The resulting customized Area Quantification analysis module was then applied to all subsequent images analyzed. Following HALO analysis completion, the ‘Integrated Optical Density (OD)’ for that section image was calculated as the product of the average DAB intensity across all pixels in the annotation contour (optical density) multiplied by the percentage of the DAB stained area. The average value for the ‘Integrated OD’ of all section images for each analyzed brain were tabulated in order to compare HALO image analysis results between groups of animals dosed with different antibody variants. The analysis performed on an exemplary brain is shown in FIGS. 1A-1C.

IHC staining for human anti-IgG-kappa was used to measure the amount of test material in the mouse brain in order to assess the efficiency with which the antibody variants entered the brain parenchyma. Because the animals were perfused post-dosing, the unbound/floating antibody was cleared prior to tissue-processing and only bound antibody was detected via the above-described IHC method. This method provided a measure of IHC staining intensity and allowed a direct comparison of the intensity of the IHC signal across animals dosed with different antibody variants across many studies in order to rank them.

E. Immunostaining for pERK in Mouse Brains. Brain slices were labelled overnight at room temperature with 1:5000 rabbit anti-phosphorylated extracellular signal-regulated kinase ½ antibody (anti-pERK ½) (Neuromics RA15002) to reveal pERK1/2, a biomarker downstream of the TrkB pathway. All incubation solutions from the primary antibody onward used Tris buffered saline (TBS) (Fisher Scientific BP247-1) diluted to 1×, and Triton X100 (Promega H5141). Following rinses, a biotinylated goat anti-rabbit IgG (Vector Laboratories BA-1000-1.5) secondary antibody was applied at a dilution of 1:1000. After further rinses, sections were treated with VECTASTAIN® Elite ABC-HRP Kit (Vector Laboratories PK-6100) following manufacturer's standard protocol. Sections were rinsed with TBS and then treated with 3,3′-diaminobenzidine tetrahydrochloride (DAB), hydrogen peroxide and nickel (Sigma, cat #N4882). After another TBS rinse, sections were mounted on gelatin coated glass slides, and air dried. Slides were not counterstained and only dehydrated in alcohols and cleared in xylene. Sections were coverslipped prior to imaging.

For pERK1/2 analysis, an annotation contour was drawn along the margins of the hippocampal formation. The HALO™ tissue classifier module was applied to the images, which allowed separation of the tissue in the image from non-tissue classes (i.e., glass) based on artificial intelligence random forest algorithm (Breiman L., et al, 2001), trained during a learning session on several representative images. The resulting Tissue-Glass classifier parameters were then applied to all subsequent images analyzed. The Area Quantification module of HALO™ was used to quantify the intensity of the nickel-intensified 3,3′-diaminobenzidine (NiDAB) chromogen staining within the tissue portion of the selected hippocampus images. Similar to the Tissue-Glass classifier, parameters for the Area Quantification module were established over a training session on a few representative images in order to teach the algorithm to categorizing the NiDAB-only IHC staining signal as ‘weak,’ ‘moderate’, or ‘strong’ based on pixel density. In order to quantify the cell-specific IHC signal only, areas in the Cornu Ammonis 3 (CA3) region where intense fiber staining was present were manually excluded from the analysis by using the HALO™ Exclusion Pen tool.

The resulting customized Area Quantification analysis module was then applied to all subsequent images analyzed. Following HALO™ analysis completion, the ‘Integrated Optical Density (OD)’ for that section image was calculated as the sum of the total nickel intensity across all pixels in the annotation contour. The average value for the ‘Integrated OD’ of all section images for each analyzed brain were tabulated, in order to compare HALO™ image analysis results between groups of animals dosed with different test antibodies. Quantified data was graphed in Prism 9 software (GraphPad Software, Inc., La Jolla, CA, U.S.A).

F. Enzyme-linked immunosorbent assay (ELISA) measuring human IgG protein in mouse serum and CSF. Antibody concentrations in murine serum and cerebral spinal fluid (CSF) were measured with a human IgG ELISA kit (Abcam, ab212169). In brief, the concentration of antibody test material were measured by spectrometry and these samples were used as standards. Standards and test samples were diluted so that the antibody concentration was below 15 ng/mL and loaded at 50 μL/well into 96-well plates coated with primary antibody provided by the kit. Kit secondary antibodies were then added to each well, and plates were sealed and incubated at room temperature for 40 minutes on a plate shaker. Plates were washed and TMB was added as a chromogen at 100 μL/well. Sealed plates were incubated at room temperature for 5 minutes on a plate shaker to develop the color reaction. The intensity of the color reaction was assessed by absorbance at a wavelength of 450 nm. Sample protein concentrations were determined by interpolating the blank control absorbance values subtracted against the respective antibody standard curve with a second order polynomial model.

### Example 2: Quantification of Human Immunoglobulins Found in Mouse Brains Expressing a Human FcRn Transgene after In Vivo Administration

To model how hIgG1 antibodies localize into human brain, the quantity of hIgG1 antibodies found in the mouse brain after in vivo administration was investigated in Tg276 mice. The heavy chain variable (V_{H}) [SEQ ID NO: 1] and light chain variable (V_{L}) regions [SEQ ID NO: 2] of a biomarker antibody raised against the 04 mouse oligodendrocyte lineage expressed in the brain were respectively grafted to the heavy chain constant region (C_{H}) [SEQ ID NO: 3] and kappa constant region (C_{L}) [SEQ ID NO: 4] of human immunoglobulin G1 (hIgG1) in a mammalian expression vector using standard techniques. Variant antibodies were produced using the methods in Example 1 by replacing the C_{H} region of the antibodies with the sequences provided in Table 5, except the terminal lysine (K447) for each sequence listed in Table 5 was deleted during the cloning process. A variant of the IgG heavy chain with a deletion of the terminal lysine (ΔK447) was used in this experiment to improve the homogeneity of the protein product; however, the presence or absence of the K447 residue was not expected to modulate the translocation of the antibody across the blood brain barrier. Variant antibody proteins without the ΔK447 mutation were also used to investigate the quantity of antibodies in the brain after in vivo administration in Example 11.

Variants of this anti-O4 antibody listed in Table 5 were cloned, expressed in Expi293 cells, purified, and administered i.v. to 7-8 week old Tg276 mice at 30 mg/kg. Forty-eight hours after antibody administration, animals were sacrificed and perfused with PBS. Mouse brains were dissected, fixed, embedded, sectioned, and immunostained for the kappa chain of hIgG1. The quantity of antibody in the brain parenchyma of each variant was quantified as the integrated OD. These methods were described in further detail in Example 1, sections A, C, and D.

Table 5 shows the integrated OD of fifteen variants tested in this manner. The variants are identified by the substitutions on the hIgG1 Fc domain, with each substitution presented in the format:
original residue in single letter amino acid format-residue number according to EU numbering-new residue in single amino acid format
   and, where substitutions at multiple residues were introduced, each individual residue change was separated by a “/”. The residue number referenced by each variant identification can be found on FIG. 2, which uses the EU numbering system originally described by Edelman et al., Proc Natl Acad Sci USA. 1969 May; 63(1): 78-85. The “wild type” antibody bearing the unmodified hIgG1 heavy chain [SEQ ID NO: 5] and kappa light chain [SEQ ID NO:7] was denoted as WT. Each antibody variant was expressed with the light chain described by SEQ ID NO: 7 and a heavy chain comprising a V_{H} with the sequence of SEQ ID NO: 1 and a C_{H} selected from the C_{H} sequences listed in Table 5. The substitutions made in the C_{H} region targeted the Fragment Crystallizable (Fc) region of the C_{H}.

TABLE 5
Ranked Integrated OD of anti-O4 hIgG1 antibody
Fc variants in the brains of Tg276 mice

| | C_{H} SEQ | Integrated |
|---|---|---|
| Antibody Variant | ID NO: | OD |
| M252Y/S254T/T256E/V308P/N434W | 36 | 38.928 |
| M252Y/N286E/V308P/M428I/N434Y | 37 | 30.481 |
| M252Y/V308P/N434W | 39 | 25.957 |
| M252Y/V308P/N434Y | 40 | 20.675 |
| M252Y/T256E/V308P/N434W | 41 | 20.276 |
| M252Y/S254T/T256E/N434W | 42 | 17.308 |
| M252Y/T256E/V308P | 44 | 12.154 |
| M252F/V308P | 46 | 4.495 |
| WT | 47 | 2.594 |
| M252Y/S254T/T256E/V308W | 48 | 0.008 |

As shown in Table 5, little immunoreactivity to the anti-O4 hIgG1 antibody was found in the brains of Tg276 animals tested with the wild type anti-O4 hIgG1 antibody. However, in contrast, anti-O4 hIgG1 antibody immunoreactivity was over 10-fold greater than WT in the brains of animals tested with certain variants, including M252Y/S254T/T256E/V308P/N434W; M252Y/N286E/V308P/M428I/N434Y; and M252Y/V308P/N434W.

### Example 3: Quantity of Human Immunoglobulin Variants Found in Mouse Brain after In Vivo Administration to Mice Expressing a Human FcRn Transgene

To further characterize how various antibody substitutions in the hIgG1 Fc domain might affect the extent to which these antibody variants enter the brain parenchyma, targeted mutagenesis techniques were used to introduce additional modifications in the Fc region of anti-O4 antibodies at or near residues found to modulate antibody penetration into the brain as described in Example 2. Antibody variants listed in Table 6 were cloned, expressed, and purified using the methods described in Example 1. Each antibody variant was expressed with the kappa light chain described by SEQ ID NO: 7 and a heavy chain comprising a V_{H} with a sequence of SEQ ID NO: 1 and a C_{H} sequence listed in Table 6, except the terminal lysine (K447) for each sequence listed in Table 6 was deleted during the cloning process. The antibody variants were administered to Tg276 mice, and the integrated OD for hIgG1 immunostaining was quantified in the brains collected from these mice as described in Example 1. The WT control immunostaining result was the same as described in Example 2.

TABLE 6
Ranked Integrated OD of additional hIgG1
antibody variants in Tg276 mice

| Antibody Variant | C_{H} SEQ ID NO: | Integrated OD |
|---|---|---|
| M252Y/S254T/T256E/V308P | 51 | 50.607 |
| M252Y/V308P | 52 | 39.634 |
| M252Y/S254T/V308P | 53 | 22.125 |
| S254T/T256E/V308P/N434W | 54 | 19.381 |
| M252Y/S254T/V308P/N434W | 55 | 14.912 |
| N434G | 57 | 7.338 |
| H435N | 58 | 6.204 |
| M252Y | 59 | 6.094 |
| T256E/V308P | 60 | 6.015 |
| V308N | 61 | 5.085 |
| V308F | 62 | 5.015 |
| M252Y/S254T/T256E | 63 | 4.711 |
| H433T | 64 | 4.635 |
| S254T/T256E/V308P | 65 | 4.621 |
| H435K | 66 | 4.473 |
| M252W | 67 | 4.036 |
| H433E | 68 | 3.923 |
| M252Y/S254T/T256E/V308S | 69 | 3.912 |
| V308P | 70 | 3.840 |
| M252Y/S254T/T256E/V308A | 71 | 3.796 |
| N434F | 72 | 3.773 |
| R255Y | 73 | 3.749 |
| M252Y/S254T/T256E/V308I | 74 | 3.618 |
| H433Q | 75 | 3.450 |
| N434Q | 76 | 3.384 |
| M252Y/S254T/T256E/V308F | 77 | 3.304 |
| S254T/V308P | 78 | 3.279 |
| N434A | 79 | 3.273 |
| N434L | 80 | 3.200 |
| H433L | 81 | 3.087 |
| P257A | 82 | 3.001 |
| V308M | 83 | 2.922 |
| H433P | 84 | 2.909 |
| V308I | 85 | 2.783 |
| H435R | 86 | 2.590 |
| M252Y/S254T/T256E/V308M | 87 | 2.536 |
| N434M | 88 | 2.507 |
| H433N | 89 | 2.426 |
| N434H | 90 | 2.310 |
| N434V | 91 | 2.245 |
| N434I | 92 | 2.083 |
| M252Y/S254T/T256E/V308G | 93 | 2.064 |
| M252F | 94 | 1.903 |
| M252Y/S254T/T256E/V308T | 95 | 1.862 |
| V308G | 96 | 1.841 |
| N434S | 97 | 1.649 |
| H433R | 98 | 1.632 |
| H433Y | 99 | 1.585 |
| M252Y/S254T/T256E/V308L | 100 | 1.549 |
| N434K | 101 | 1.544 |
| V308A | 102 | 1.521 |
| V308E | 103 | 1.512 |
| M252G | 104 | 1.492 |
| N434Y | 105 | 1.443 |
| V308H | 106 | 1.235 |
| H433I | 107 | 1.178 |
| R255G | 108 | 1.154 |
| H433C | 109 | 1.113 |
| V308S | 110 | 1.108 |
| M252L | 111 | 1.086 |
| M252A | 112 | 1.060 |
| M252K | 113 | 1.059 |
| V308T | 114 | 1.015 |
| V308Q | 115 | 0.986 |
| H433D | 116 | 0.982 |
| M252E | 117 | 0.931 |
| N434T | 118 | 0.851 |
| H433A | 119 | 0.808 |
| M252H | 120 | 0.791 |
| N434R | 121 | 0.785 |
| M252R | 122 | 0.774 |
| H433V | 123 | 0.712 |
| N434E | 124 | 0.704 |
| V308L | 125 | 0.691 |
| N434W | 126 | 0.552 |
| M252Y/S254T/T256E/V308R | 127 | 0.544 |
| M252N | 128 | 0.509 |
| M252Y/S254T/T256E/V308Q | 129 | 0.468 |
| M252Q | 130 | 0.332 |
| H435L | 131 | 0.296 |
| N434D | 132 | 0.295 |
| H435D | 133 | 0.294 |
| H435G | 134 | 0.287 |
| N434P | 135 | 0.273 |
| H435E | 136 | 0.268 |
| V308D | 137 | 0.238 |
| M252V | 138 | 0.204 |
| M252Y/S254T/T256E/V308K | 139 | 0.182 |
| H435Q | 140 | 0.153 |
| M252I | 141 | 0.150 |
| M252Y/S254T/T256E/V308D | 142 | 0.146 |
| M252T | 143 | 0.137 |
| M252D | 144 | 0.092 |
| H435I | 145 | 0.073 |
| M252P | 146 | 0.062 |
| V308W | 147 | 0.061 |
| V308R | 148 | 0.058 |
| M252Y/S254T/T256E/V308N | 149 | 0.043 |
| M252Y/S254T/T256E/V308E | 150 | 0.037 |
| M252Y/S254T/T256E/V308H | 151 | 0.030 |
| V308K | 152 | 0.015 |
| V308Y | 153 | 0.013 |
| M252Y/S254T/T256E/V308Y | 154 | 0.002 |

As shown in Table 6, several additional antibody modifications increased the quantity of hIgG1 in the brain when compared to wild type hIgG1 antibody. While individual amino acid substitutions in the Fc domain had limited effects on the penetration of human immunoglobulin protein across the blood brain barrier and into the brain, the introduction of multiple substitutions into the same Fc domain in some cases could result in unexpected synergistic increase on antibody penetration into the brain parenchyma. For example, while a single substitution at M252Y or V308P each increased the amount of antibody found in the brain parenchyma by 2 to 3-fold over wild type Fc, the combination of M252Y/V308P together increased the amount of antibody partitioning into the brain parenchyma by more than 15-fold. Additional substitutions at one or more of N434, S254 and T256 were able to further increase or decrease the amount of antibody found in the brain parenchyma.

### Example 4: Screening Human Immunoglobulin Variants Produced by Random Mutagenesis Using an Antibody Internalization Assay

Without wishing to be limited by any proposed mechanism of action, in an attempt to identify additional antibody variants that efficiently enter the brain parenchyma, it was hypothesized that antibodies with increased transcytosis through the blood-brain barrier (BBB) would be partitioned into the brain parenchyma more efficiently. In order for an antibody to be transcytosed through the BBB, it must first be internalized by brain endothelial cells. Because JEG3 human endothelial placental choriocarcinoma cells are endothelial cells of a lineage similar to the endothelial cells of human brain capillary blood vessels, we reasoned that JEG3 cells stably induced with lentiviral particles harboring a human FCGRT transgene would serve as a good model for transcytosis through the BBB as described in Example 1, part B.

Therefore, as described in section A of Example 1, antibody variants of O4 hIgG1 (containing the heavy chain and light chain variable regions from SEQ ID NO: 1 and 2 respectively) were generated by mutagenesis targeted to its CH domain (SEQ ID NO: 3) while keeping the kappa light chain constant region unchanged (SEQ ID NO: 4). Each antibody variant was expressed with the light chain described by SEQ ID NO: 7 and a heavy chain comprising SEQ ID NO: 1 and the CH Seq ID listed in Table 7. The variants were expressed in Expi293 in 96-well blocks, and the supernatants were tested in the Antibody Internalization Assay. The amount of internalized antibody was quantified and compared to the internalization of the M252Y/S254T/T256E/V308P/N434W variant. This variant was chosen as a positive control because it was able to efficiently partition into the brain parenchyma in Example 2.

Table 7 lists the Fc variants tested in this assay. In total, 581 variants were tested and, of these, 141 variants were found to be internalized by JEG3 cells at least 10-fold more efficiently than wild type antibodies. Amongst these antibodies, there was a trend toward antibodies which bore two or more amino acid substitutions selected from the group consisting of M252Y, V308P, N434W, N434Y, N434F, and N434H.

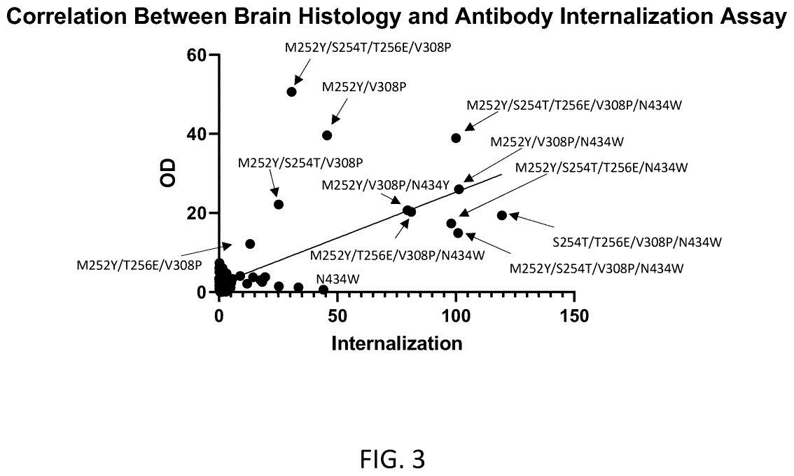

Eighty of the 581 variants screened in this assay were also tested in Tg276 mice as described in Examples 2 and 3. For these 80 variants, the amount of antibody detected in the mouse brains (as measured by OD) was compared to the proportion of each variant that was internalized on the Antibody Internalization Assay described in FIG. 3.

Typically, antibody variants that were internalized by JEG3 cells at least 10-fold more efficiently than wildtype antibodies were also detected at high levels in the brain of Tg276 mice, suggesting that these antibodies were also more efficiently translocated across the blood brain barrier.

TABLE 7
Ranked Internalization of anti-O4 hIgG1 antibody variants
generated by random mutagenesis in JEG3 cells

| | C_{H} SEQ | % |
|---|---|---|
| Antibody Variant | ID NO: | Internalization |
| M252Y/S254R/T256I/V308P/N434Y | 155 | 134.00 |
| M252Y/S254E/T256P/V308W | 156 | 120.46 |
| S254T/T256E/V308P/N434W | 54 | 119.48 |
| M252Y/T256V/V308P/N434W | 157 | 115.44 |
| M252Y/S254T/T256P/V308P/N434Y | 158 | 102.09 |
| M252Y/S254T/T256E/V308P/N434Y | 6 | 101.84 |
| M252Y/V308P/N434W | 39 | 101.25 |
| M252Y/S254T/V308P/N434W | 55 | 100.96 |
| M252Y/T256D/V308P/N434W | 159 | 100.17 |
| M252Y/S254T/T256E/V308P/N434W | 36 | 100.00 |
| V308P/N434W | 160 | 98.92 |
| M252Y/S254T/T256E/N434W | 42 | 98.05 |
| M252Y/T256S/V308P/N434W | 161 | 97.31 |
| M252Y/T256L/V308P/N434W | 162 | 97.12 |
| M252Y/S254T/T256L/V308P/N434Y | 163 | 96.00 |
| M252Y/T256V/V308P/N434F | 164 | 95.40 |
| M252Y/T256P/V308W | 165 | 95.33 |
| M252Y/T256Y/V308P/N434W | 166 | 93.75 |
| M252Y/S254T/V308P/N434Y | 700 | 93.15 |
| M252Y/T256H/V308P/N434W | 167 | 92.70 |
| M252Y/V308P/N434F | 19 | 92.21 |
| M252Y/T256P/V308P/N434W | 168 | 90.95 |
| M252Y/S254A/T256D/V308P/N434Y | 169 | 89.24 |
| M252Y/S254V/T256P/V308P/N434Y | 170 | 89.00 |
| M252Y/T256K/V308P/N434W | 171 | 88.45 |
| M252Y/S254A/T256V/V308P/N434Y | 172 | 87.00 |
| M252Y/S254I/T256R/V308P/N434Y | 173 | 87.00 |
| M252Y/T256M/V308P/N434W | 174 | 86.50 |
| M252Y/S254T/T256E/V308P/N434F | 405 | 86.10 |
| M252Y/T256G/V308P/N434P | 175 | 86.06 |
| M252Y/T256R/V308P/N434W | 176 | 84.76 |
| M252Y/S254T/V308P/N434F | 43 | 83.88 |
| M252Y/S254A/T256A/V308P/N434Y | 177 | 83.00 |
| M252Y/T256E/V308P/N434W | 41 | 81.10 |
| M252Y/T256A/V308P/N434W | 178 | 81.07 |
| M252Y/S254G/T256V/V308P/N434Y | 179 | 80.00 |
| M252Y/V308P/N434Y | 40 | 79.66 |
| M252Y/S254A/T256N/V308P/N434Y | 180 | 77.00 |
| M252Y/S254T/V308P/N434H | 45 | 76.69 |
| M252Y/S254Y/T256Q/V308P/N434Y | 181 | 76.00 |
| M252Y/T256G/V308P/N434W | 182 | 75.17 |
| M252Y/S254R/T256R/V308P/N434Y | 183 | 75.00 |
| M252Y/S254L/T256E/V308P/N434Y | 184 | 73.00 |
| M252Y/V308P/N434H | 269 | 71.76 |
| M252Y/T256E/V308P/N434F | 185 | 70.75 |
| M252Y/T256I/V308P/N434W | 186 | 70.52 |
| M252Y/T256W/V308P/N434W | 187 | 69.97 |
| M252Y/S254T/T256E/V308P/N434H | 603 | 69.93 |
| M252Y/T256Y/V308P/N434H | 188 | 65.84 |
| M252Y/S254G/T256E/V308P/N434Y | 189 | 65.21 |
| M252Y/T256I/V308P/N434Y | 190 | 64.82 |
| M252Y/T256R/V308P/N434Y | 191 | 64.24 |
| M252Y/S254V/T256S/V308P/N434Y | 192 | 63.29 |
| M252Y/T256P/V308P/N434F | 193 | 62.40 |
| M252Y/S254G/T256A/V308P/N434Y | 194 | 60.00 |
| M252Y/S254H/V308P/N434Y | 195 | 60.00 |
| M252Y/S254R/T256S/V308P/N434Y | 196 | 59.00 |
| M252Y/S254L/T256V/V308P/N434Y | 197 | 58.63 |
| M252Y/S254I/T256E/V308P/N434Y | 198 | 57.33 |
| M252Y/S254H/T256D/V308P/N434Y | 199 | 56.77 |
| S254T/T256E/V308P/N434Y | 654 | 56.36 |
| M252Y/T256V/V308P/N434Y | 200 | 56.16 |
| M252Y/S254H/T256V/V308P/N434Y | 201 | 55.59 |
| M252Y/S254L/T256S/V308P/N434Y | 202 | 55.16 |
| M252Y/S254A/T256L/V308P/N434Y | 203 | 55.00 |
| M252Y/T256A/V308P/N434F | 204 | 54.84 |
| M252Y/T256H/V308P/N434P | 205 | 54.75 |
| M252Y/T256K/V308P/N434Y | 206 | 54.68 |
| M252Y/T256E/V308P/N434Y | 207 | 54.65 |
| M252Y/T256N/V308P/N434Y | 208 | 54.15 |
| M252Y/T256G/V308P/N434F | 209 | 54.09 |
| M252Y/S254N/T256G/V308P/N434Y | 210 | 54.00 |
| M252Y/T256W/V308P/N434Y | 211 | 53.67 |
| M252Y/S254G/T256G/V308P/N434Y | 212 | 53.61 |
| M252Y/S254T/T256A/V308P/N434Y | 213 | 53.49 |
| S254T/T256E/V308P/N434F | 655 | 53.48 |
| M252Y/S254V/T256D/V308P/N434Y | 214 | 53.41 |
| M252Y/T256I/V308P/N434V | 215 | 52.38 |
| M252Y/S254F/V308P/N434Y | 216 | 52.00 |
| M252Y/T256E/V308P/N434H | 217 | 51.62 |
| M252Y/T256P/V308P/N434Y | 218 | 51.48 |
| M252Y/S254A/T256S/V308P/N434Y | 219 | 50.54 |
| M252Y/T256S/V308P/N434Y | 220 | 50.12 |
| M252Y/S254I/T256V/V308P/N434Y | 221 | 50.00 |
| M252Y/T256S/V308P/N434F | 222 | 49.69 |
| M252Y/T256D/V308P/N434Y | 223 | 49.54 |
| M252Y/T256S/V308P/N434H | 224 | 49.54 |
| M252Y/S254R/T256E/V308P/N434Y | 225 | 49.00 |
| M252Y/S254T/T256M/V308P/N434Y | 226 | 49.00 |
| M252Y/T256Y/V308P/N434Y | 227 | 48.80 |
| M252Y/T256S/V308P/N434G | 228 | 48.50 |
| M252Y/S254A/T256R/V308P/N434Y | 229 | 48.23 |
| M252Y/T256V/V308P/N434G | 230 | 47.98 |
| M252Y/T256H/V308P/N434Y | 231 | 47.87 |
| M252Y/T256V/V308P/N434M | 232 | 47.07 |
| M252Y/S254A/T256P/V308P/N434Y | 233 | 46.62 |
| M252Y/S254Q/T256L/V308P/N434Y | 234 | 46.00 |
| M252Y/V308P | 52 | 45.63 |
| M252Y/T256P/V308P/N434A | 235 | 45.25 |
| M252Y/T256F/V308P/N434W | 236 | 45.01 |
| M252Y/S254G/T256L/V308P/N434Y | 237 | 45.00 |
| M252Y/S254G/T256Q/V308P/N434Y | 238 | 45.00 |
| M252Y/T256G/V308P/N434Y | 239 | 44.30 |
| M252Y/S254G/T256S/V308P/N434Y | 240 | 44.24 |
| N434W | 126 | 44.14 |
| M252Y/T256A/V308P/N434Y | 241 | 44.06 |
| M252Y/T256V/V308P/N434T | 242 | 44.02 |
| M252Y/S254G/T256R/V308P/N434Y | 243 | 44.00 |
| M252Y/S254V/T256I/V308P/N434Y | 244 | 43.21 |
| M252Y/T256R/V308P/N434S | 245 | 43.09 |
| M252Y/S254L/T256R/V308P/N434Y | 246 | 42.36 |
| M252Y/T256L/V308P/N434F | 247 | 42.27 |
| S254W | 248 | 41.66 |
| M252Y/S254G/T256W/V308P/N434Y | 249 | 41.14 |
| M252Y/S254R/T256Q/V308P/N434Y | 250 | 41.12 |
| M252Y/S254L/T256D/V308P/N434Y | 251 | 41.00 |
| M252Y/S254R/T256N/V308P/N434Y | 252 | 41.00 |
| M252Y/S254I/T256I/V308P/N434Y | 253 | 40.36 |
| M252Y/T256F/V308P/N434Y | 254 | 40.35 |
| M252Y/S254A/T256E/V308P/N434Y | 255 | 40.30 |
| M252Y/S254M/T256R/V308P/N434Y | 256 | 40.00 |
| M252Y/S254V/T256N/V308P/N434Y | 257 | 40.00 |
| M252Y/T256D/V308P | 258 | 39.90 |
| M252Y/S254G/T256N/V308P/N434Y | 259 | 39.00 |
| M252Y/T256F/V308P/N434F | 260 | 38.90 |
| M252Y/S254A/T256K/V308P/N434Y | 261 | 38.13 |
| M252Y/T256G/V308P/N434H | 262 | 37.84 |
| S254F | 263 | 37.51 |
| M252Y/S254G/V308P/N434Y | 264 | 37.43 |
| M252Y/T256D/V308P/N434T | 265 | 36.95 |
| M252Y/T256Q/V308P/N434W | 266 | 35.45 |
| M252Y/T256E/V308P/N434A | 267 | 34.39 |
| M252Y/S254L/T256G/V308P/N434Y | 268 | 34.00 |
| M252Y/T256Q/V308P/N434Y | 270 | 33.68 |
| R255G | 108 | 33.50 |
| M252Y/T256L/V308P/N434Y | 271 | 33.27 |
| M252Y/T256G/V308P/N434R | 272 | 33.05 |
| M252Y/S254E/T256P/V308P/N434Y | 273 | 33.00 |
| M252Y/S254I/T256M/V308P/N434Y | 274 | 33.00 |
| M252Y/T256P/V308P/N434H | 275 | 32.80 |
| M252Y/S254A/V308P/N434Y | 276 | 31.96 |
| M252Y/S254L/T256A/V308P/N434Y | 277 | 31.82 |
| M252Y/S254T/T256Y/V308P/N434Y | 278 | 31.62 |
| M252Y/T256A/V308P/N434S | 279 | 31.49 |
| M252Y/T256H/V308P/N434S | 280 | 31.30 |
| M252Y/S254Q/T256V/V308P/N434Y | 281 | 31.00 |
| M252Y/T256I/V308P/N434T | 282 | 30.86 |
| M252Y/S254T/T256E/V308P | 51 | 30.66 |
| M252Y/S254V/T256K/V308P/N434Y | 283 | 30.45 |
| M252Y/T256G/V308P/N434K | 284 | 30.09 |
| M252Y/T256W/V308P | 285 | 29.09 |
| M252Y/T256R/V308P/N434A | 286 | 28.25 |
| M252Y/T256S/V308P/N434K | 287 | 27.71 |
| M252Y/V308P/N434S | 288 | 27.29 |
| M252Y/T256R/V308P/N434V | 289 | 26.17 |
| M252Y/T256Q/V308P | 290 | 25.74 |
| M252Y/T256G/V308P/N434S | 291 | 25.62 |
| N434Y | 105 | 25.27 |
| M252Y/S254T/V308P | 53 | 25.21 |
| M252Y/T256L/V308P/N434I | 292 | 25.18 |
| M252Y/V308F | 293 | 24.89 |
| M252Y/T256V/V308P/N434R | 294 | 24.45 |
| M252Y/T256S/V308P/N434T | 295 | 24.36 |
| M252Y/T256P/V308P/N434G | 296 | 24.13 |
| M252Y/T256E/V308P/N434G | 297 | 22.05 |
| M252Y/T256V/V308P/N434I | 298 | 21.71 |
| M252Y/T256H/V308P | 299 | 21.58 |
| M252I/V308S | 300 | 20.63 |
| M252Y/T256P/V308P/N434I | 301 | 20.56 |
| M252Y/T256E/V308P/N434S | 302 | 19.85 |
| M252Y/T256N/V308P | 303 | 19.56 |
| N434F | 72 | 19.47 |
| S254T/T256E/V308P/N434H | 656 | 18.93 |
| M252Y/T256R/V308P/N434G | 304 | 18.91 |
| M252Y/T256K/V308P/N434G | 305 | 18.46 |
| M252Y/S254T/T256E/V308M | 87 | 18.21 |
| M252Y/T256W/V308P/N434S | 306 | 18.20 |
| M252Y/T256D/V308P/N434S | 307 | 17.89 |
| P257A | 82 | 17.57 |
| M252Y/T256Q/V308P/N434L | 308 | 17.42 |
| M252Y/T256E/V308P/N434R | 309 | 17.37 |
| M252Y/V308P/N434A | 310 | 16.76 |
| M252Y/T256F/V308P/N434S | 311 | 16.58 |
| M252Y/T256K/V308P/N434S | 312 | 16.51 |
| M252Y/V308P/N434R | 313 | 15.97 |
| M252Y/T256E/V308P/N434Q | 314 | 15.63 |
| M252Y/V308P/N434M | 315 | 15.42 |
| M252Y/T256G/V308P/N434M | 316 | 15.16 |
| M252Y/T256W/V308P/N434V | 317 | 15.03 |
| M252Y/T256Y/V308P/N434S | 318 | 14.49 |
| M252Y/T256P/V308P/N434K | 319 | 14.44 |
| R255Y | 73 | 14.36 |
| M252Y/T256S/V308P/N434S | 320 | 14.23 |
| M252Y/T256R/V308P/N434I | 321 | 13.81 |
| M252Y/T256D/V308P/N434P | 322 | 13.79 |
| M252Y/T256Y/V308P/N434V | 323 | 13.58 |
| M252Y/T256L/V308P | 324 | 13.38 |
| M252Y/T256E/V308P | 44 | 13.09 |
| M252Y/V308P/N434P | 325 | 12.79 |
| M252Y/T256L/V308P/N434K | 326 | 12.74 |
| M252Y/T256G/V308P/N434Q | 327 | 12.59 |
| M252Y/T256N/V308P/N434K | 328 | 12.50 |
| M252Y/V308P/N434G | 329 | 12.36 |
| M252Y/T256P/V308P/N434M | 330 | 12.31 |
| M252Y/T256F/V308P/N434R | 331 | 12.25 |
| M252Y/S254T/T256E/V308G | 93 | 11.91 |
| M252Y/V308P/N434Q | 332 | 11.72 |
| M252Y/T256G/V308P | 333 | 10.79 |
| M252Y/T256D/V308P/N434A | 334 | 10.77 |
| M252Y/T256I/V308P/N434I | 335 | 10.36 |
| M252Y/T256E/V308P/N434P | 336 | 10.33 |
| R255E | 337 | 10.23 |
| M252Y/T256Y/V308P/N434R | 338 | 9.64 |
| M252Y/T256I/V308P | 339 | 9.49 |
| M252Y/T256R/V308P/N434K | 340 | 9.41 |
| M252W | 67 | 8.99 |
| M252Y/T256R/V308P | 341 | 8.77 |
| M252Y/T256I/V308P/N434P | 342 | 8.71 |
| M252Y/T256E/V308P/N434I | 343 | 8.62 |
| M252Y/V308K | 344 | 8.48 |
| M252Y/T256Y/V308P | 345 | 8.42 |
| M252Y/T256F/V308P | 346 | 8.27 |
| M252S/V308A | 347 | 7.98 |
| M252Y/T256E/V308P/N434T | 348 | 7.83 |
| M252Y/T256L/V308P/N434S | 349 | 7.60 |
| M252T/V308H | 350 | 7.59 |
| M252Y/T256M/V308P | 351 | 7.36 |
| M252Y/T256S/V308P/N434Q | 352 | 7.30 |
| M252V/V308H | 353 | 7.11 |
| M252Y/T256Q/V308P/N434R | 354 | 6.58 |
| M252Y/V308P/N434T | 355 | 6.56 |
| M252Y/T256N/V308P/N434Q | 356 | 6.42 |
| M252N/V308K | 357 | 6.41 |
| M252W/V308G | 358 | 6.39 |
| M252Y/T256I/V308P/N434D | 359 | 6.33 |
| M252T/V308W | 360 | 6.04 |
| M252Y/V308A | 361 | 6.01 |
| M252Y/V308E | 362 | 5.72 |
| M252Y/S254T/T256E/V308F | 77 | 5.62 |
| M252K/V308K | 363 | 5.45 |
| M252Y/T256K/V308P/N434L | 364 | 5.39 |
| M252F/V308F | 365 | 5.26 |
| M252Y/T256N/V308P/N434L | 366 | 5.25 |
| N434H | 90 | 5.16 |
| M252Y/T256W/V308P/N434M | 367 | 5.15 |
| M252Y/V308L | 368 | 5.11 |
| M252Y/V308P/N434I | 369 | 4.98 |
| M252K | 113 | 4.91 |
| M252R/V308P | 370 | 4.89 |
| M252Y/T256R/V308P/N434L | 371 | 4.88 |
| M252Y/T256Q/V308P/N434K | 372 | 4.81 |
| M252Y/T256I/V308P/N434M | 373 | 4.79 |
| M252W/V308S | 374 | 4.54 |
| M252E/V308W | 375 | 4.51 |
| M252Y/T256I/V308P/N434R | 376 | 4.44 |
| M252H/V308M | 377 | 4.36 |
| T256S | 378 | 4.26 |
| M252W/V308H | 379 | 4.19 |
| P257I | 380 | 4.12 |
| M252Y/T256A/V308P/N434K | 381 | 4.03 |
| M252A/V308M | 382 | 3.95 |
| M252N/V308L | 383 | 3.92 |
| M252Y/T256R/V308P/N434R | 384 | 3.76 |
| M252A/V308F | 385 | 3.75 |
| M252Y/T256Y/V308P/N434T | 386 | 3.66 |
| R255H | 387 | 3.56 |
| M252Y/T256L/V308P/N434L | 388 | 3.42 |
| M252Y/T256A/V308P/N434L | 389 | 3.40 |
| M252Y/S254T/T256E/V308Q | 129 | 3.37 |
| M252Y/T256R/V308P/N434Q | 390 | 3.34 |
| M252I/V308A | 391 | 3.32 |
| M252Y/T256I/V308P/N434L | 392 | 3.32 |
| M252Q/V308W | 393 | 3.31 |
| M252Y/T256F/V308P/N434D | 394 | 3.31 |
| P257S | 395 | 3.30 |
| V308M | 83 | 3.25 |
| M252Y/T256L/V308P/N434T | 396 | 3.24 |
| M252I | 141 | 3.20 |
| M252Y/T256F/V308P/N434L | 397 | 3.17 |
| M252Y/S254T/T256E | 63 | 3.10 |
| M252L/V308W | 398 | 3.02 |
| WT | 47 | 3.00 |
| M252Y/T256L/V308P/N434P | 399 | 2.99 |
| M252Y/S254T/T256E/V308A | 71 | 2.94 |
| M252Y/T256E/V308P/N434K | 400 | 2.91 |
| M252Y/T256F/V308P/N434E | 401 | 2.91 |
| M252Y/T256E/V308P/N434L | 402 | 2.90 |
| V308W | 147 | 2.90 |
| T256D | 403 | 2.85 |
| M252Y/T256A/V308P | 404 | 2.84 |
| M252E/V308L | 406 | 2.75 |
| M252Y/T256A/V308P/N434I | 407 | 2.74 |
| M252F/V308W | 408 | 2.73 |
| M252F/V308H | 409 | 2.62 |
| P257N | 410 | 2.57 |
| M252G/V308S | 411 | 2.53 |
| M252S/V308F | 412 | 2.51 |
| M252Y/V308P/N434D | 413 | 2.51 |
| M252H/V308W | 414 | 2.50 |
| M252Y/T256L/V308P/N434R | 415 | 2.45 |
| M252Y/S254T/T256E/V308R | 127 | 2.42 |
| M252Y/T256A/V308P/N434H | 416 | 2.39 |
| M252Y/T256E/V308P/N434E | 417 | 2.37 |
| M252F/V308D | 418 | 2.27 |
| V308F | 62 | 2.25 |
| M252Y/T256D/V308P/N434E | 419 | 2.24 |
| M252Y/T256G/V308P/N434L | 420 | 2.23 |
| M252R/V308F | 421 | 2.22 |
| M252A/V308W | 422 | 2.21 |
| M252V/V308A | 423 | 2.20 |
| M252Y/T256G/V308P/N434I | 424 | 2.20 |
| M252F/V308K | 425 | 2.19 |
| T256M | 426 | 2.16 |
| M252W/V308R | 427 | 2.15 |
| M252Y/V308P/N434L | 428 | 2.13 |
| M252A/V308R | 429 | 2.12 |
| M252F | 94 | 2.07 |
| M252Q/V308S | 430 | 2.05 |
| M252G | 104 | 2.00 |
| M252P/V308F | 431 | 1.98 |
| M252Y/S254T/T256E/V308K | 139 | 1.98 |
| M252E/V308F | 432 | 1.90 |
| M252H/V308N | 433 | 1.85 |
| M252Y/T256G/V308P/N434D | 434 | 1.82 |
| M252Y/V308I | 435 | 1.79 |
| M252T | 143 | 1.77 |
| M252L | 111 | 1.73 |
| M252Y/T256D/V308P/N434R | 436 | 1.72 |
| M252V | 138 | 1.68 |
| M252A/V308L | 437 | 1.65 |
| M252P | 146 | 1.59 |
| M252Q/V308F | 438 | 1.58 |
| V308A | 102 | 1.57 |
| M252H/V308P | 439 | 1.56 |
| M252Y/T256V/V308P/N434L | 440 | 1.54 |
| M252V/V308S | 441 | 1.51 |
| M252D/V308P | 442 | 1.47 |
| M252Y/T256E | 443 | 1.47 |
| M252F/V308E | 444 | 1.45 |
| M252G/V308L | 445 | 1.45 |
| M252W/V308F | 446 | 1.45 |
| M252P/V308M | 447 | 1.44 |
| M252I/V308Y | 448 | 1.43 |
| T256E/V308P | 60 | 1.43 |
| M252Y/T256R/V308P/N434F | 449 | 1.40 |
| M252V/V308L | 450 | 1.39 |
| M252T/V308S | 451 | 1.37 |
| M252A/V308N | 452 | 1.32 |
| M252N/V308E | 453 | 1.29 |
| M252Y/T256G/V308P/N434V | 454 | 1.27 |
| M252W/V308N | 455 | 1.25 |
| M252Q/V308Y | 456 | 1.23 |
| M252A | 112 | 1.21 |
| M252F/V308I | 457 | 1.19 |
| M252I/V308F | 458 | 1.19 |
| M252N | 128 | 1.18 |
| M252H/V308R | 459 | 1.17 |
| M252Y/T256D/V308P/N434H | 460 | 1.16 |
| M252Y/S254T/T256E/V308H | 151 | 1.15 |
| M252L/V308F | 461 | 1.13 |
| V308L | 125 | 1.13 |
| M252I/V308R | 462 | 1.12 |
| M252E/V308D | 463 | 1.11 |
| M252F/V308T | 464 | 1.11 |
| M252K/V308R | 465 | 1.11 |
| M252V/V308F | 466 | 1.11 |
| M252Y/T256R/V308P/N434D | 467 | 1.09 |
| M252Y/V308P/N434K | 468 | 1.08 |
| S254T/T256E/V308P | 65 | 1.08 |
| M252D | 144 | 1.06 |
| M252G/V308F | 469 | 1.06 |
| M252H | 120 | 1.06 |
| M252Y/S254T/T256E/V308I | 74 | 1.06 |
| M252Q/V308L | 470 | 1.03 |
| M252Y/T256E/V308P/N434D | 471 | 1.03 |
| N434V | 91 | 1.03 |
| M252H/V308F | 472 | 1.02 |
| M252V/V308M | 473 | 1.01 |
| M252E | 117 | 1.00 |
| V308H | 106 | 1.00 |
| M252I/V308N | 474 | 0.98 |
| M252Y/T256D/V308P/N434I | 475 | 0.98 |
| M252I/V308M | 476 | 0.97 |
| M252S/V308E | 477 | 0.96 |
| M252P/V308E | 478 | 0.92 |
| M252Y/T256A/V308P/N434G | 479 | 0.92 |
| V308P | 70 | 0.92 |
| M252Y/T256Q/V308P/N434P | 480 | 0.89 |
| M252Y/T256S/V308P/N434D | 481 | 0.87 |
| M252R/V308L | 482 | 0.86 |
| M252Y/T256P/V308P/N434P | 483 | 0.85 |
| M252K/V308D | 484 | 0.84 |
| M252K/V308N | 485 | 0.84 |
| M252Y/T256W/V308P/N434I | 486 | 0.84 |
| M252E/V308R | 487 | 0.83 |
| M252I/V308T | 488 | 0.83 |
| M252T/V308K | 489 | 0.83 |
| M252Y/T256K/V308P/N434E | 490 | 0.83 |
| T256N | 491 | 0.80 |
| M252S/V308Y | 492 | 0.80 |
| M252A/V308S | 493 | 0.77 |
| M252D/V308S | 494 | 0.76 |
| M252Y/T256F/V308P/N434P | 495 | 0.76 |
| M252V/V308E | 496 | 0.75 |
| M252D/V308H | 497 | 0.73 |
| M252S | 498 | 0.73 |
| M252E/V308N | 499 | 0.72 |
| M252Y/T256S/V308P/N434E | 500 | 0.72 |
| M252P/V308D | 501 | 0.71 |
| V308I | 85 | 0.70 |
| M252L/V308Y | 502 | 0.69 |
| M252R | 122 | 0.69 |
| S254F/V308W | 503 | 0.64 |
| M252H/V308A | 504 | 0.63 |
| M252R/V308A | 505 | 0.63 |
| M252Y/V308Y | 506 | 0.63 |
| M252I/V308D | 507 | 0.62 |
| M252R/V308R | 508 | 0.62 |
| M252D/V308R | 509 | 0.61 |
| M252H/V308D | 510 | 0.60 |
| M252T/V308P | 511 | 0.60 |
| M252E/V308S | 512 | 0.59 |
| M252I/V308H | 513 | 0.58 |
| M252I/V308W | 514 | 0.58 |
| M252L/V308D | 515 | 0.58 |
| T256C | 516 | 0.57 |
| M252Y/V308W | 517 | 0.57 |
| T256Y | 518 | 0.56 |
| M252E/V308Y | 519 | 0.56 |
| M252Y/T256P/V308P/N434L | 520 | 0.55 |
| R255V | 521 | 0.54 |
| M252W/V308D | 522 | 0.53 |
| S254V | 523 | 0.53 |
| M252Y/V308H | 524 | 0.51 |
| V308Y | 153 | 0.50 |
| M252R/V308Y | 525 | 0.49 |
| T256I | 526 | 0.49 |
| M252G/V308Q | 527 | 0.48 |
| T256K | 528 | 0.48 |
| M252T/V308E | 530 | 0.46 |
| M252V/V308R | 531 | 0.46 |
| M252D/V308Y | 532 | 0.45 |
| M252I/V308E | 533 | 0.45 |
| M252T/V308N | 534 | 0.45 |
| M252Y/T256M/V308P/N434E | 535 | 0.45 |
| M252Y/V308P/N434E | 536 | 0.45 |
| N434A | 79 | 0.45 |
| N434L | 80 | 0.44 |
| M252E/V308H | 537 | 0.43 |
| N434P | 135 | 0.43 |
| M252V/V308Y | 538 | 0.41 |
| M252R/V308E | 539 | 0.40 |
| M252R/V308N | 540 | 0.40 |
| M252Q/V308R | 541 | 0.38 |
| M252V/V308D | 542 | 0.38 |
| M252Q/V308K | 543 | 0.37 |
| R255A | 544 | 0.35 |
| M252D/V308F | 545 | 0.35 |
| M252R/V308S | 546 | 0.35 |
| M252Y/T256V/V308P/N434D | 547 | 0.35 |
| T256H | 548 | 0.34 |
| M252L/V308L | 549 | 0.33 |
| M252I/V308I | 550 | 0.32 |
| T256V | 551 | 0.31 |
| T256A | 552 | 0.30 |
| S254L | 553 | 0.30 |
| M252G/V308Y | 554 | 0.29 |
| M252I/V308P | 555 | 0.29 |
| M252Q | 130 | 0.29 |
| M252Y/T256S/V308P/N434P | 556 | 0.29 |
| P257T | 557 | 0.28 |
| R255C | 558 | 0.28 |
| M252F/V308Y | 559 | 0.28 |
| V308T | 114 | 0.28 |
| M252P/V308H | 560 | 0.27 |
| M252D/V308N | 561 | 0.26 |
| M252Y/T256H/V308P/N434A | 562 | 0.26 |
| M252Y/T256F/V308P/N434G | 563 | 0.25 |
| T256L | 564 | 0.25 |
| N434G | 57 | 0.24 |
| M252K/V308L | 566 | 0.24 |
| V308D | 137 | 0.23 |
| N434C | 567 | 0.23 |
| M252H/V308I | 568 | 0.22 |
| M252K/V308Y | 569 | 0.22 |
| M252R/V308I | 570 | 0.22 |
| M252S/V308W | 571 | 0.22 |
| V308K | 152 | 0.21 |
| R255T | 572 | 0.21 |
| S254Y | 573 | 0.20 |
| T256Q | 574 | 0.20 |
| S254M | 575 | 0.20 |
| P257Y | 576 | 0.19 |
| T256E | 577 | 0.19 |
| M252K/V308S | 578 | 0.19 |
| M252Y/T256V/V308P/N434P | 579 | 0.19 |
| V308Q | 115 | 0.19 |
| V308S | 110 | 0.19 |
| M252A/V308Y | 580 | 0.18 |
| M252I/V308G | 581 | 0.18 |
| M252K/V308F | 582 | 0.18 |
| M252Y/T256H/V308P/N434F | 583 | 0.18 |
| M252Y/T256P/V308P/N434R | 584 | 0.18 |
| P257D | 585 | 0.17 |
| V308G | 96 | 0.17 |
| P257H | 586 | 0.17 |
| M252G/V308H | 587 | 0.16 |
| M252W/V308E | 588 | 0.16 |
| M252Y/T256S/V308P/N434A | 589 | 0.16 |
| M252Y/T256S/V308P/N434R | 590 | 0.15 |
| M252P/V308G | 591 | 0.14 |
| M252Y/T256Y/V308P/N434E | 592 | 0.14 |
| S254H | 593 | 0.14 |
| M252Y/S254T/T256E/V308D | 142 | 0.14 |
| R255S | 594 | 0.13 |
| M252E/V308Q | 595 | 0.13 |
| N434S | 97 | 0.13 |
| M252N/V308A | 596 | 0.12 |
| M252V/V308G | 597 | 0.12 |
| V308E | 103 | 0.12 |
| N434M | 88 | 0.12 |
| M252N/V308P | 598 | 0.11 |
| M252S/V308D | 599 | 0.11 |
| M252S/V308K | 600 | 0.11 |
| M252T/V308F | 601 | 0.11 |
| P257M | 602 | 0.10 |
| T256W | 604 | 0.10 |
| M252A/V308E | 605 | 0.10 |
| M252S/V308L | 606 | 0.10 |
| M252S/V308R | 607 | 0.10 |
| S254M/V308W | 608 | 0.10 |
| V308N | 61 | 0.10 |
| P257F | 609 | 0.10 |
| S254K | 610 | 0.10 |
| N434E | 124 | 0.09 |
| M252H/V308G | 611 | 0.09 |
| M252Q/V308N | 612 | 0.09 |
| M252Y | 59 | 0.09 |
| S254T/V308P | 78 | 0.09 |
| M252P/V308S | 613 | 0.08 |
| V308R | 148 | 0.08 |
| R255W | 614 | 0.08 |
| T256G | 615 | 0.07 |
| P257G | 616 | 0.07 |
| N434Q | 76 | 0.07 |
| M252P/V308W | 617 | 0.07 |
| M252Y/T256R/V308P/N434E | 618 | 0.07 |
| R255L | 619 | 0.06 |
| N434D | 132 | 0.06 |
| M252D/V308D | 620 | 0.06 |
| S254I/V308W | 621 | 0.06 |
| M252H/V308E | 622 | 0.05 |
| M252Y/T256S/V308P/N434L | 623 | 0.05 |
| S254P | 624 | 0.05 |
| R255D | 625 | 0.05 |
| R255K | 626 | 0.04 |
| R255Q | 627 | 0.04 |
| M252F/V308R | 628 | 0.04 |
| M252R/V308D | 629 | 0.04 |
| M252V/V308Q | 630 | 0.04 |
| S254L/V308W | 631 | 0.04 |
| P257R | 632 | 0.04 |
| M252N/V308D | 633 | 0.03 |
| M252T/V308Q | 634 | 0.03 |
| V308C | 635 | 0.03 |
| P257W | 636 | 0.03 |
| P257C | 637 | 0.03 |
| P257K | 638 | 0.03 |
| P257E | 639 | 0.02 |
| T256F | 640 | 0.02 |
| N434T | 118 | 0.02 |
| N434R | 121 | 0.02 |
| S254I | 641 | 0.02 |
| M252Q/V308D | 642 | 0.02 |
| M252T/V308T | 643 | 0.02 |
| M252Y/T256G/V308P/N434T | 644 | 0.02 |
| S254Y/V308W | 645 | 0.02 |
| R255M | 646 | 0.02 |
| P257L | 647 | 0.01 |
| M252G/V308D | 648 | 0.01 |
| T256R | 649 | 0.01 |
| P257Q | 650 | 0.01 |
| M252D/V308E | 651 | 0.00 |
| M252I/V308Q | 652 | 0.00 |
| R255F | 653 | 0.00 |
| R255I | 49 | 0.00 |
| R255N | 50 | 0.00 |

Even though the correlation between the Antibody Internalization Assay and the Human IgG Kappa Light Chain Immunostaining was high, the JEG3 cell Internalization assay was not perfectly predictive of translocation results in the humanized mouse brain. For example, we observed that variants comprising both the M252Y and the V308P substitutions were detected in the mouse brain at levels greater than predicted by the Internalization assay, while variants that comprised the N434W substitution were detected in the mouse brain at levels less than predicted by the Internalization assay. This was likely because although internalization is required for transcytosis, other cellular factors may still further influence the transport of the antibody variant through the endothelium. In all likelihood, the M252Y and V308P substitutions interact with intracellular transport factors favorably, while the N434W substitution does not.

Thus, although the Human IgG Kappa Light Chain Immunostaining Assay is a better model of human physiology, the Antibody Internalization Assay served as a high throughput screen for antibody variants that could efficiently cross the blood-brain barrier in vivo.

### Example 5: Quantity of Human Immunoglobulin Variants Identified by an Antibody Internalization Assay Found in the Brains of Mice Expressing a Human FcRn Transgene after In Vivo Administration

The Antibody Internalization Assay as described in Example 4 identified several antibody variants that were highly internalized by JEG3 cells. Additional experiments were performed in Tg276 mice to quantify selected variants in the mouse brain following intravenous administration. The Fc substitutions from selected variants were incorporated into the 04 parent antibody sequence comprising the heavy and light chain variable regions described respectively in SEQ ID NOs: 1 and 2. Each antibody variant was expressed with the light chain described by SEQ ID NO: 7 and a heavy chain comprising SEQ ID NO: 1 and the C_{H} Seq ID listed in Table 8, except that the terminal lysine (K447) for each sequence listed in Table 8 was deleted during the cloning process. These antibody proteins were expressed in and purified from Expi293 cells as described in Example 1. The antibody variants were then administered intravenously to Tg276 mice, and the integrated OD of immunostained hIgG1 was quantified in the brains collected from these mice as described in parts C and D of Example 1.

TABLE 8
Ranked quantity of certain hIgG1 antibody variants identified
by an antibody internalization assay in the brains
of Tg276 mice after in vivo administration

| | C_{H} SEQ | Integrated |
|---|---|---|
| Antibody Variant | ID NO: | OD |
| M252Y/S254T/T256E/V308P/N434H | 603 | 48.10 |
| M252Y/S254T/V308P/N434H | 45 | 47.43 |
| M252Y/T256V/V308P/N434F | 164 | 32.66 |
| M252Y/S254T/T256E/V308P/N434F | 405 | 34.34 |
| M252Y/T256E/V308P/N434H | 217 | 31.59 |
| M252Y/S254T/T256E/V308P/N434Y | 6 | 31.52 |
| M252Y/T256E/V308P/N434W | 41 | 30.23 |
| M252Y/T256S/V308P/N434W | 161 | 29.12 |
| M252Y/T256W/V308P/N434Y | 211 | 25.65 |
| M252Y/T256E/V308P/N434F | 185 | 23.33 |
| M252Y/T256R/V308P/N434Y | 191 | 22.87 |
| M252Y/T256P/V308P/N434W | 168 | 21.64 |
| M252Y/T256E/V308P/N434Y | 207 | 20.50 |
| M252Y/S254T/V308P/N434W | 55 | 19.49 |
| M252Y/T256F/V308P/N434F | 260 | 19.11 |
| M252Y/T256W/V308P/N434W | 187 | 16.41 |
| M252Y/T256F/V308P/N434Y | 254 | 15.99 |
| M252Y/T256L/V308P/N434W | 162 | 14.50 |
| M252Y/T256Q/V308P/N434W | 266 | 13.67 |
| M252Y/T256E/V308P/N434W | 41 | 13.59 |
| M252Y/T256A/V308P/N434W | 178 | 12.98 |
| M252Y/T256E/V308P/N434P | 336 | 11.92 |
| M252Y/T256V/V308P/N434W | 157 | 11.85 |
| M252Y/T2561/V308P/N434Y | 190 | 11.50 |
| M252Y/T256R/V308P/N434W | 176 | 11.38 |
| M252Y/T256G/V308P/N434Y | 239 | 10.50 |
| M252Y/T256L/V308P/N434Y | 271 | 10.24 |
| M252Y/T256V/V308P/N434Y | 200 | 9.75 |
| M252Y/T256Y/V308P/N434Y | 227 | 9.07 |
| M252Y/T256N/V308P/N434Y | 208 | 9.05 |
| M252Y/T256Q/V308P/N434Y | 270 | 8.73 |
| M252Y/T256A/V308P/N434Y | 241 | 6.36 |
| M252Y/T256P/V308P/N434Y | 218 | 6.04 |
| M252Y/T256S/V308P/N434Y | 220 | 5.16 |
| M252Y/T256D/V308P/N434W | 159 | 5.05 |
| M252Y/T256H/V308P/N434F | 583 | 4.62 |
| WT | 50 | 2.49 |

As shown in Table 8, all of the selected antibody variants tested entered the brain parenchyma more efficiently than a corresponding antibody with a wildtype Fc region. Consistent with Example 4, the M252Y and V308P substitutions increase the quantity of human immunoglobulin proteins in the brain of Tg276 mice after in vivo administration. A histidine, phenylalanine, tyrosine, or tryptophan substitution at N434 further increased the quantity of immunoglobulin proteins that entered the brain parenchyma. While substitutions at T256 were well-tolerated, in general substitutions at this position did not substantially increase or decrease the quantity of antibody proteins in the brain following intravenous administration.

### Example 6: Quantification of Antibody Proteins Comprising a Range of Different Variable Regions and Either M252Y/V308P or M252Y/S254T/T256E/V308P/N434W Substitutions Found in the Brain of Mice Expressing a Human FcRn Transgene

Substitutions in the Fc domain were demonstrated in earlier examples to enhance blood brain barrier penetration in an anti-O4 IgG1 system. In this example a range of substitutions were tested in a range of different human antibody isotypes and/or with a variety of different variable region sequences to investigate whether the substitutions or antibody isotype or antibody variable region influence how an antibody entered the brain parenchyma.

The VH regions of the different antibodies, each of which were not specifically targeted to brain antigens were each separately fused to the CH region of the human (i) IgG1 [SEQ ID NO: 3], (ii) IgG2 [SEQ ID NO: 25], or (iii) IgG4 [SEQ ID NO: 26], and the VL of each of these antibodies fused to either the CL of the human kappa chain (SEQ ID NO: 4) or the CL of the human lambda chain (SEQ ID NO: 35) using the methods described in Example 1, section A. For each antibody, two variants were made to the Fc domain, corresponding to either the M252Y/V308P variant or the M252Y/S254T/T256E/V308P/N434W variant. Although the CH sequences listed in Table 9 include lysine 447, this terminal residue was truncated during the cloning process for each of these variants for the purposes of this experiment. The antibodies were then expressed in Expi293 cells, purified from supernatant, and administered to Tg276 mice as described in Example 2. The integrated OD of hIgG1 immunostaining was quantified in the brains collected from these mice. The antibody variable region sequences used were designated Ab1 (described in U.S. Pat. No. 6,258,562)[VH SEQ ID NO: 9; VL SEQ ID NO: 10]; Ab2 (described in U.S. Pat. No. 7,364,736) [VH SEQ ID NO: 11; VL SEQ ID NO: 12]; Ab3 (described in U.S. Pat. No. 6,355,245) [VH SEQ ID NO: 13; VL SEQ ID NO: 14]; Ab4 (described in WO 2006/121168)[VH SEQ ID NO: 15; VL SEQ ID NO: 16]; Ab5 (Muller et al., Structure. 6:1153 (1998)) [VH SEQ ID NO: 17; VL SEQ ID NO: 18]); and Ab7 (as described in U.S. Pat. No. 7,138,501) [VH SEQ ID NO: 701; VL SEQ ID NO: 702].

TABLE 9
Intergrated OD of hIgG1 antibody variants comprising different variable regions

| | V_{H} | V_{L} | C_{L} | | | C_{H} | |
|---|---|---|---|---|---|---|---|
| | SEQ | SEQ | SEQ | | | SEQ | |
| Variable | ID | ID | ID | Human | | ID | Integrated |
| Region | NO: | NO: | NO | Isotype | Fc Substitution | NO: | OD |
| Ab1 | 9 | 10 | 4 | IgG1 | WT | 3 | 0.73 |
| Ab1 | 9 | 10 | 4 | IgG1 | M252Y/V308P | 27 | 5.08 |
| Ab1 | 9 | 10 | 4 | IgG1 | M252Y/S254T/T256E/V308P/N434W | 28 | 1.24 |
| Ab1 | 9 | 10 | 4 | IgG2 | WT | 25 | 4.36 |
| Ab1 | 9 | 10 | 4 | IgG2 | M252Y/V308P | 29 | 7.72 |
| Ab1 | 9 | 10 | 4 | IgG2 | M252Y/S254T/T256E/V308P/N434W | 31 | 21.26 |
| Ab1 | 9 | 10 | 4 | IgG4 | WT | 26 | 13.01 |
| Ab1 | 9 | 10 | 4 | IgG4 | M252Y/V308P | 30 | 32.28 |
| Ab1 | 9 | 10 | 4 | IgG4 | M252Y/S254T/T256E/V308P/N434W | 32 | 0.64 |
| Ab2 | 11 | 12 | 4 | IgG1 | WT | 3 | 1.04 |
| Ab2 | 11 | 12 | 4 | IgG1 | M252Y/V308P | 27 | 13.24 |
| Ab2 | 11 | 12 | 4 | IgG1 | M252Y/S254T/T256E/V308P/N434W | 28 | 3.05 |
| Ab2 | 11 | 12 | 4 | IgG2 | WT | 25 | 3.35 |
| Ab2 | 11 | 12 | 4 | IgG2 | M252Y/V308P | 29 | 17.49 |
| Ab2 | 11 | 12 | 4 | IgG2 | M252Y/S254T/T256E/V308P/N434W | 31 | 5.70 |
| Ab2 | 11 | 12 | 4 | IgG4 | WT | 26 | 6.37 |
| Ab2 | 11 | 12 | 4 | IgG4 | M252Y/V308P | 30 | 12.08 |
| Ab2 | 11 | 12 | 4 | IgG4 | M252Y/S254T/T256E/V308P/N434W | 32 | 27.08 |
| Ab3 | 13 | 14 | 4 | IgG1 | WT | 3 | 0.65 |
| Ab3 | 13 | 14 | 4 | IgG1 | M252Y/V308P | 27 | 7.76 |
| Ab3 | 13 | 14 | 4 | IgG1 | M252Y/S254T/T256E/V308P/N434W | 28 | 2.38 |
| Ab3 | 13 | 14 | 4 | IgG2 | WT | 25 | 1.67 |
| Ab3 | 13 | 14 | 4 | IgG2 | M252Y/V308P | 29 | 1.44 |
| Ab3 | 13 | 14 | 4 | IgG2 | M252Y/S254T/T256E/V308P/N434W | 31 | 44.74 |
| Ab3 | 13 | 14 | 4 | IgG4 | WT | 26 | 6.93 |
| Ab3 | 13 | 14 | 4 | IgG4 | M252Y/V308P | 30 | 4.82 |
| Ab3 | 13 | 14 | 4 | IgG4 | M252Y/S254T/T256E/V308P/N434W | 32 | 19.62 |
| Ab4 | 15 | 16 | 4 | IgG1 | WT | 3 | 1.89 |
| Ab4 | 15 | 16 | 4 | IgG1 | M252Y/V308P | 27 | 10.50 |
| Ab4 | 15 | 16 | 4 | IgG1 | M252Y/S254T/T256E/V308P/N434W | 28 | 12.24 |
| Ab4 | 15 | 16 | 4 | IgG2 | WT | 25 | 4.59 |
| Ab4 | 15 | 16 | 4 | IgG2 | M252Y/V308P | 29 | 2.69 |
| Ab4 | 15 | 16 | 4 | IgG2 | M252Y/S254T/T256E/V308P/N434W | 31 | 25.41 |
| Ab4 | 15 | 16 | 4 | IgG4 | WT | 26 | 2.61 |
| Ab4 | 15 | 16 | 4 | IgG4 | M252Y/V308P | 30 | 18.31 |
| Ab4 | 15 | 16 | 4 | IgG4 | M252Y/S254T/T256E/V308P/N434W | 32 | 1.51 |
| Ab5 | 17 | 18 | 4 | IgG1 | WT | 3 | 0.78 |
| Ab5 | 17 | 18 | 4 | IgG1 | M252Y/V308P | 27 | 3.62 |
| Ab5 | 17 | 18 | 4 | IgG1 | M252Y/S254T/T256E/V308P/N434W | 28 | 1.77 |
| Ab5 | 17 | 18 | 4 | IgG2 | WT | 25 | 1.92 |
| Ab5 | 17 | 18 | 4 | IgG2 | M252Y/V308P | 29 | 2.18 |
| Ab5 | 17 | 18 | 4 | IgG2 | M252Y/S254T/T256E/V308P/N434W | 31 | 24.07 |
| Ab5 | 17 | 18 | 4 | IgG4 | WT | 26 | 2.94 |
| Ab5 | 17 | 18 | 4 | IgG4 | M252Y/V308P | 30 | 7.59 |
| Ab5 | 17 | 18 | 4 | IgG4 | M252Y/S254T/T256E/V308P/N434W | 32 | 2.54 |
| Ab7 | 701 | 702 | 35 | IgG1 | WT | 3 | 17.15 |
| Ab7 | 701 | 702 | 35 | IgG1 | M252Y/V308P | 27 | 102.48 |
| Ab7 | 701 | 702 | 35 | IgG1 | M252Y/S254T/T256E/V308P/N434W | 28 | 50.42 |

As shown in Table 9, antibodies comprising M252Y/V308P and M252Y/S254T/T256E/V308P/N434W substitutions in their Fc region localized to the brain at higher levels compared to WT antibodies, regardless of the variable regions or isotype and, accordingly, the amount of blood brain barrier penetration was not substantially altered following changes to the antibody variable region or antibody isotype.

### Example 7: Pharmacodynamics of hIgG1 Fc-Modified Variants in Tg276 Mice

Experiments were carried out to assess BBB transport by antibody variants comprising substitutions in the Fc domain of CH (SEQ ID NO: 3) corresponding to the M252Y/S254T/T256E/V308P/N434W (SEQ ID: 36) variant. The antibodies were constructed using variable regions of the light and heavy chains of an antibody that activates Tropomyosin receptor kinase B (TrkB; WO 2010/086828A2) (LC: SEQ ID 34; HC: SEQ ID 33) fused to the kappa chain constant region (SEQ ID NO: 4) and the M252Y/S254T/T256E/V308P/N434W (SEQ ID: 36) or the WT (SEQ ID: 47) C_{H} variant region using the methods described in part A of Example 1. Adult male transgenic Tg276 mice received a single 10 mg/kg intravenous (IV) administration of either one of the anti-TrkB antibody variants or of an isotype control antibody raised against the keyhole limpet hemocyanin (KLH) antigen.

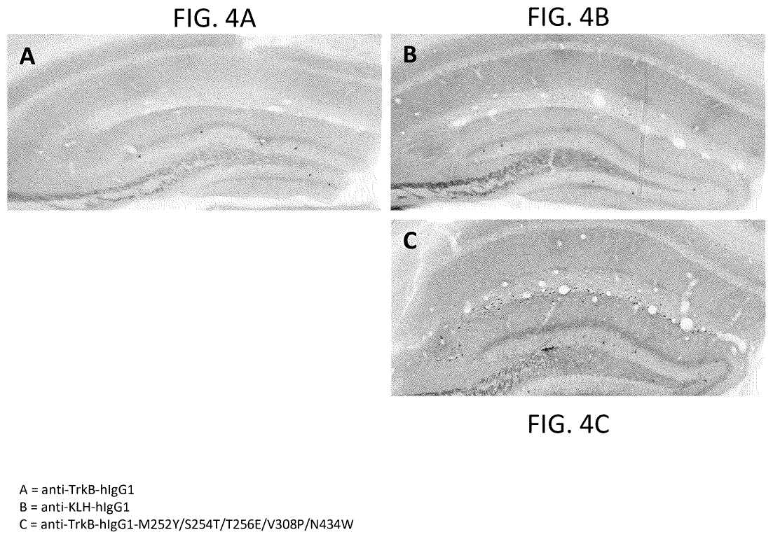

Forty-eight hours after antibody administration animals were sacrificed and the brains were prepared for immunohistochemical analysis as described in parts C, D, and E of Example 1. Activation of the TrkB receptor in mouse brain by the administered antibody was detected by increased staining for pERK resulting from phosphorylation of Extracellular Signal-Regulated Kinase downstream of TrkB signaling. As shown in FIG. 4, activity was detected as darkly stained regions on the mouse brain pERK immunostained sections.

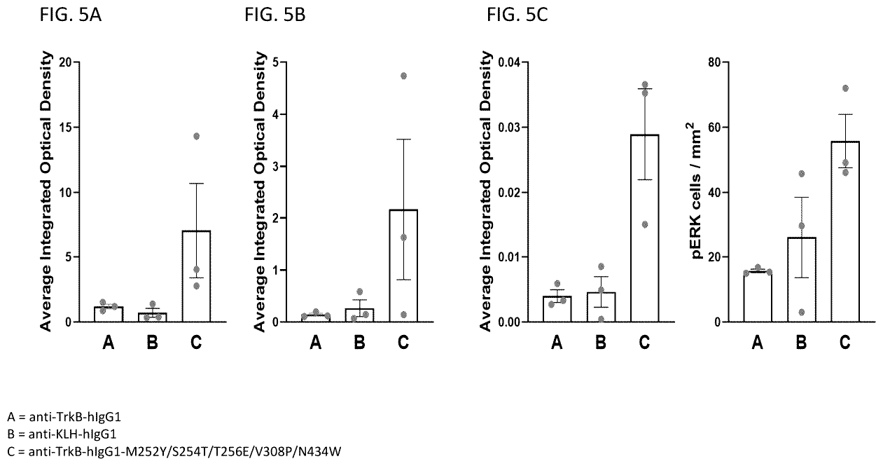

Antibodies comprising the Fc domain substitutions M252Y/S254T/T256E/V308P/N434W were more efficiently transported across the BBB than antibodies comprising wild type Fc domains (FIG. 5A). This increased transport was observed particularly in the hippocampus (FIG. 5B), a region where TrkB signaling is known to occur. The increased transport of the agonist TrkB antibody resulted in increased detectable functional TrkB activity in the hippocampus, as evidenced by an increase in the average integrated optical density of pERK immunostaining as well as the number of pERK-positive cells (FIG. 5C).

These results demonstrated that certain substitutions in the Fc domain of human immunoglobulin proteins improved the therapeutic activity of antibodies in the brain by enhancing the amount of these antibody proteins that are able to enter the brain following intravenous administration.

### Example 8: Pharmacokinetics of hIgG1 Fc-Modified Variants in Tg276 Mice

Pharmacokinetic (PK) assays were performed to evaluate the quantity of Fc-modified antibodies in brain, serum, and cerebrospinal fluid (CSF) samples of adult male transgenic Tg276 mice. The WT and M252Y/S254T/T256E/V308P/N434W variants of the anti-O4 antibody used in Example 2 were prepared, and mice in the study received a single 30 mg/kg intravenous (IV) injection of test antibody. The WT antibody comprised SEQ ID Nos: 1, 2, 3, and 4, comprising in order the antibody VH, Vk, CH, and CL regions respectively. The M252Y/S254T/T256E/V308P/N434W variant comprised SEQ ID Nos.: 1, 2, 36, and 4. Animals were sacrificed, and the brain, serum, and CSF tissue were collected at various intervals following administration. The quantity of the test anti-O4 antibody in the brain was estimated by sectioning and immunostaining the brain samples. The concentration of the anti-O4 antibody in the plasma and CSF was estimated by the ELISA assay described in Example 1 (F).

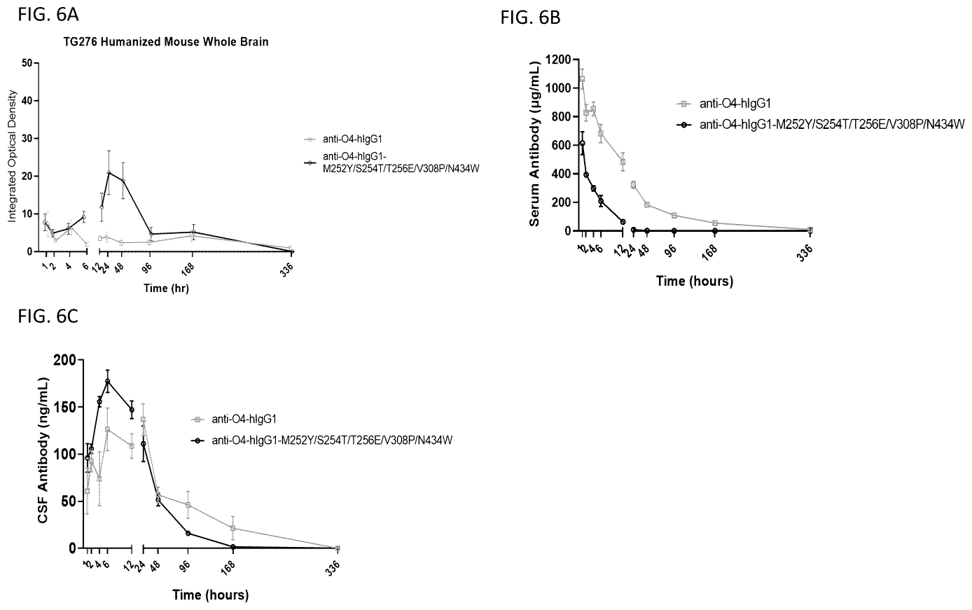

While it was difficult to detect the WT antibody in the brain parenchyma at any of time points tested, the M252Y/S254T/T256E/V308P/N434W variant was found in the brain at high levels shortly after dosing (FIG. 6A). The amount of antibody in the brain reached a maximum level 24 h after dosing and returned to baseline levels four days after dosing. When the optical density values between the M252Y/S254T/T256E/V308P/N434W variant and the WT variant were compared using a t-test it was found that the brains of mice dosed with the M252Y/S254T/T256E/V308P/N434W variant contained significantly more human IgG test antibody compared to those of mice dosed with the WT variant (p<0.001).

The efficiency by which M252Y/S254T/T256E/V308P/N434W entered the brain parenchyma was different from its behavior in serum but was mirrored by its behavior in CSF. Although the M252Y/S254T/T256E/V308P/N434W variant persisted in the serum for a shorter time than that of the WT variant (FIG. 6B), greater amounts of the M252Y/S254T/T256E/V308P/N434W variant entered the CSF (FIG. 6C). After dosing, the M252Y/S254T/T256E/V308P/N434W variant antibody began to enter into the CSF, peaking at 6 hours after dosing. This antibody was then gradually eliminated from the CSF over the next two weeks. The elimination of the M252Y/S254T/T256E/V308P/N434W variant from the CSF was faster than that of the WT variant.

### Example 9: Antibody Transcytosis Assay

Previous groups have reported that in vitro transcytosis assays testing antibodies on endothelial cells grown on Transwell® membranes can be used to model how well antibody proteins can cross the BBB (WO 2020/132230). To compare our results, an in vitro assay to compare and rank antibody transcytosis activity was developed based on a combination of methods that rely on pH-dependent interactions of antibodies with FcRn and an assessment of FcRn-mediated intracellular trafficking of antibodies under physiological conditions.

MDCK II cells (ECACC 00062107) were maintained in DMEM basal medium supplemented with 10% fetal bovine serum, 5 mM L-glutamine, 175 μg/ml hygromycin, 0.9 mg/mL Geneticin and stably transfected to express human FcRn [SEQ ID NO: 21] and human B2M (beta-2-microglobulin) [SEQ ID NO: 22]. Transfected cells were seeded on a permeable membrane support plate (Transwell® 0.4 μm-polyester membrane pore, Corning Inc.) for 5 days to allow for polarization of the cell monolayer. On Day 5, the integrity of tight junctions of the MDCK II cell monolayer in the membrane support plate was determined using an EVOM Epithelial Voltohmmeter (World Precision Instruments) to measure electrical resistance. Monolayers exhibiting an electrical resistance in the range of 300-450 ohms were used for transcytosis experiments.

On Day 5, the media in the apical compartment was replaced with fresh media adjusted to pH 6.0 with hydrochloric acid and test antibody was added to this compartment to a final concentration of 100 μg/mL. The media in the basolateral compartment was replaced with fresh media without adjusting its pH. Plates were incubated overnight at 37° C. in a humidified 5% CO₂ atmosphere. After 24 hours and one hour prior to the termination of the assay, Lucifer yellow (LY) was added directly to the apical compartment to assess monolayer integrity. At the end of the assay, media was collected from the apical and basolateral compartments, and the concentration of antibody in each of the two compartments was measured by ELISA.

The integrity of tight junction formation in the cell monolayer was monitored by measuring relative fluorescence units of LY passage to the basolateral compartment, relative to that of the inner chamber. Transcytosis results from wells that exhibited>0.1% of passive passage of LY in the outer chamber were disregarded. Data was normalized by dividing the concentration of each test antibody in the basolateral compartment (the amount that was transcytosed), by the concentration of a reference antibody (IAHA) that does not transcytose well [SEQ ID Nos: 7 and 23].

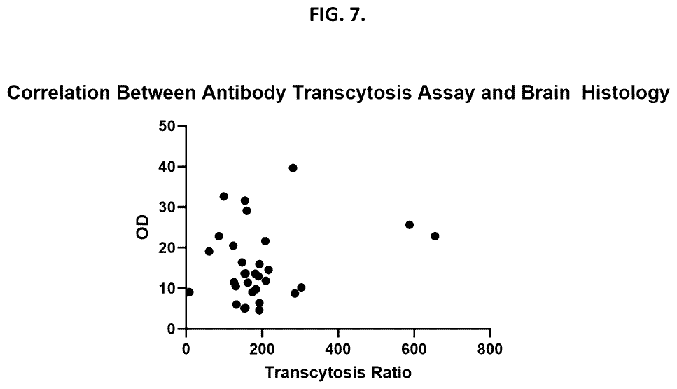

The antibodies tested in Table 10 were cloned, expressed, and purified as in Example 4. Although the CH sequences listed in Table 10 include the terminal lysine (K447), this terminal lysine was deleted during the cloning process for each of these variants for the purposes of this experiment. As shown in Table 10, the results of the Antibody Transcytosis Assay were substantially different from the results of the Antibody Internalization Assay described in Example 4. Although several of the variants identified to be efficiently internalized by the Antibody Internalization Assay were also transcytosed efficiently on the Transcytosis Assay, the Antibody Transcytosis Assay identified the M252Y/T256R/V308P/N434Y and M252Y/T256W/V308P/N434Y variants as variants that would most efficiently cross the BBB. In contrast, the Antibody Internalization Assay identified the M252Y/S254R/T256I/V308P/N434Y and M252Y/S254E/T256P/V308W variants as the best internalizers. Furthermore, the dynamic range of the Transcytosis Assay was smaller than that of the Internalization assay, as none of the antibodies tested in Table 10 were transcytosed more than 10 times more efficiently than the wildtype antibody. These results demonstrate that the Antibody Internalization Assay and the Antibody Trancytosis assay are not equivalent, and they make different predictions regarding the efficiency with which antibody variants would enter the brain parenchyma in vivo. The differing predictive values of the Antibody Internalization Assay and the Antibody Transcytosis Assay is further illustrated when each assay was compared against the brain IHC assay described in Examples 3 and 5. The normalized transcytosis value of each variant in Table 10 was plotted against the OD value of the same variant in Tables 5, 6 and 8. As shown in FIGS. 3 and 7, the Antibody Transcytosis Assay had limited predictive value compared to the Antibody Internalization Assay. Although there was a slight trend for variants that were highly transcytosed to be distributed into brain parenchyma in FIG. 7, the slope of the least squares linear regression line was not significantly different from zero. In contrast, the slope of the least squares linear regression line in FIG. 3 was significantly different from zero, and antibody variants that were demonstrated to be highly internalized and also efficiently distributed into the brain. Thus, the brain immunohistochemistry assay results which demonstrate actual transport into the brain more closely track with the Antibody Internalization Assay results, suggesting that the transcytosis assay is less useful in predicting which Fc regions will enhance transport into the central nervous system.

TABLE 10
Normalized transcytosis of certain hIgG1
antibody variants at pH 6.0 and pH 7.4

| | C_{H} SEQ | Transcytosis Normalized |
|---|---|---|
| Antibody Variant | ID NO: | to IAHA at pH 6.0 |
| M252Y/V308P | 52 | 281.1 |
| M252Y/T256L/V308P/N434Y | 271 | 302.8 |
| M252Y/T256H/V308P/N434F | 583 | 192.2 |
| M252Y/T256P/V308P/N434W | 168 | 208.3 |
| M252Y/T256F/V308P/N434F | 260 | 60.4 |
| M252Y/T256R/V308P/N434Y | 191 | 655.1 |
| M252Y/T256W/V308P/N434Y | 211 | 587.7 |
| M252Y/T256Q/V308P/N434W | 266 | 156.4 |
| M252Y/T256P/V308P/N434Y | 218 | 132.4 |
| M252Y/T256N/V308P/N434Y | 208 | 174.1 |
| M252Y/T256Y/V308P/N434Y | 227 | 8.35 |
| M252Y/T256Q/V308P/N434Y | 270 | 286 |
| M252Y/T256F/V308P/N434Y | 254 | 192.8 |
| M252Y/T256A/V308P/N434Y | 241 | 192.7 |
| M252Y/T256V/V308P/N434Y | 200 | 183.2 |
| M252Y/T256S/V308P/N434Y | 220 | 155.8 |
| M252Y/T256G/V308P/N434Y | 239 | 130.5 |
| M252Y/T2561/V308P/N434Y | 190 | 125.5 |
| M252Y/T256E/V308P/N434H | 217 | 154.6 |
| M252Y/T256E/V308P/N434Y | 207 | 123.8 |
| M252Y/T256E/V308P/N434F | 41 | 181.6 |
| M252Y/T256W/V308P/N434W | 187 | 146.7 |
| M252Y/T256V/V308P/N434F | 164 | 98.9 |
| M252Y/T256D/V308P/N434W | 159 | 153.1 |
| M252Y/T256R/V308P/N434W | 176 | 161.9 |
| M252Y/T256A/V308P/N434W | 178 | 189.8 |
| M252Y/T256V/V308P/N434W | 157 | 209.4 |
| M252Y/T256L/V308P/N434W | 162 | 216.7 |
| M252Y/T256S/V308P/N434W | 161 | 159.4 |
| M252Y/T256E/V308P/N434W | 41 | 153.6 |
| WT | 47 | 122.5 |

### Example 10: Estimating the Dissociation Constant (K(D) of the Interaction Between FcRn and Human Immunoglobulin Variants by Surface Plasmon Resonance

The interaction between human immunoglobulin proteins and FcRn is known to be affected by the pH of their environment solution. To test whether this pH-dependent interaction correlates with the quantity of antibody variants in the brain parenchyma after in vivo administration, the affinity of certain variants towards FcRn were screened at pH 6.0 and pH 7.4 on a Biacore T200 system (GE Healthcare). All experiments were conducted at 25° C. Antibodies were expressed and purified using the Expi293 expression system as described in Example 1. Cells were transfected with a heavy chain comprising the variable region of the O4 antibody used in Example 2 [SEQ ID NO: 1] fused to a heavy chain region bearing the indicated substitution in Table 11 and a light chain from Ab6 [SEQ ID NO: 24]. Although the CH sequences listed in Table 11 include the terminal lysine (K447), this terminal lysine was truncated during the cloning process for each of these variants for the purposes of this experiment. Antibody proteins were captured at a flow rate of 10 μL/min on a Protein L chip to a surface density of approximately 100 RU. Experiments conducted at pH 7.4 were conducted in PBST (1.06 mM KH₂PO₄, 155.17 mM NaCl, 2.97 mM Na₂HPO₄-7H₂O, and 0.005% Tween-20). Experiments conducted at pH 6.0 were conducted in PBST with pH adjusted to pH 6.0 with HCl. After capture of the antibody, human FcRn protein at concentrations of 0, 33, 111, 333, 1000, and 3000 nM were passed over the chip at a flow rate of 30 μL/min. The antibody-FcRn complex was allowed to dissociate for 20 s before the chip was regenerated with 10 mM glycine pH 1.7. On-rates (ka) and off-rates (kd) were estimated by the best fit to a one-to-one Langmuir binding model by simultaneous fitting of the association and dissociation sensograms. The dissociation constant (KD) for FcRn binding was then calculated as the ratio of the off rate to the on rate (kd/ka). Table 11 shows the on-rate, off-rate, and dissociation constants at pH 6.0 and pH 7.4 of antibody variants tested in this assay for hFcRn.

TABLE 11
Kinetic parameters of certain hIgG1 antibody variants at pH 6.0 and pH 7.4

| | C_{H} SEQ | pH 6.0 | | | pH 7.4 | | |
|---|---|---|---|---|---|---|---|
| Antibody Variant | ID NO: | kₐ (1/Ms) | k_{d} (1/s) | K_{D} (M) | kₐ (1/Ms) | k_{d} (1/s) | K_{D} (M) |
| M252Y/S254T/ | 36 | 1.69E+06 | 7.95E−04 | 4.71E−10 | 8.32E+03 | 1.01E−03 | 1.22E−07 |
| T256E/V308P/ | | | | | | | |
| N434W | | | | | | | |
| M252Y/S254T/ | 51 | 6.48E+05 | 1.39E−02 | 2.15E−08 | 2.01E+06 | 1.96E+00 | 9.75E−07 |
| T256E/V308P | | | | | | | |
| M252Y/N434Y/ | 38 | 1.38E+06 | 7.56E−03 | 5.48E−09 | 4.43E+04 | 1.14E+00 | 2.57E−05 |
| Y436V | | | | | | | |
| M252Y/S254T/ | 53 | 5.57E+05 | 1.77E−02 | 3.17E−08 | 1.47E+04 | 1.13E+00 | 7.67E−05 |
| V308P | | | | | | | |
| M252Y/V308P | 52 | 7.45E+05 | 2.79E−02 | 3.74E−08 | 4.26E+04 | 2.21E−03 | 5.18E−08 |
| M252Y/N286E/ | 37 | 1.84E+06 | 2.01E−04 | 1.09E−10 | 1.00E+06 | 1.03E+00 | 1.03E−06 |
| V308P/M4281/ | | | | | | | |
| N434Y | | | | | | | |
| M252Y/V308P/ | 40 | 1.61E+06 | 2.42E−03 | 1.51E−09 | 7.26E+04 | 7.48E−01 | 1.03E−05 |
| N434Y | | | | | | | |
| M252Y/V308P/ | 39 | 1.62E+06 | 1.24E−03 | 7.67E−10 | 4.26E+03 | 3.11E+00 | 7.30E−04 |
| N434W | | | | | | | |
| M252Y/T256S/ | 161 | 1.52E+06 | 1.86E−03 | 1.23E−09 | 6.68E+05 | 5.64E−02 | 8.45E−08 |
| V308P/N434W | | | | | | | |
| M252Y/T256L/ | 162 | 1.23E+06 | 3.51E−03 | 2.85E−09 | 6.34E+05 | 8.68E−02 | 1.37E−07 |
| V308P/N434W | | | | | | | |
| M252Y/T256V/ | 157 | 1.29E+06 | 2.40E−03 | 1.87E−09 | 4.76E+05 | 3.27E−02 | 6.87E−08 |
| V308P/N434W | | | | | | | |
| M252Y/S254T/ | 42 | 1.49E+06 | 6.55E−03 | 4.38E−09 | 4.45E+05 | 1.38E−01 | 3.10E−07 |
| T256E/N434W | | | | | | | |
| V308P/N434W | 160 | 1.85E+06 | 6.00E−03 | 3.25E−09 | 4.16E+05 | 1.27E−01 | 3.05E−07 |
| P257A/V308P/ | 56 | 1.66E+06 | 7.06E−03 | 4.26E−09 | 2.28E+05 | 5.45E−02 | 2.39E−07 |
| M428L/N434Y | | | | | | | |
| M252Y/T256E/ | 41 | 1.71E+06 | 1.28E−03 | 7.44E−10 | 4.86E+05 | 3.41E−02 | 7.02E−08 |
| V308P/N434W | | | | | | | |
| M252Y/T256D/ | 159 | 1.77E+06 | 2.11E−03 | 1.19E−09 | 6.50E+05 | 3.38E−02 | 5.21E−08 |
| V308P/N434W | | | | | | | |
| M252Y/T256R/ | 176 | 2.31E+06 | 1.00E−03 | 4.33E−10 | 4.10E+05 | 1.10E−01 | 2.64E−07 |
| V308P/N434W | | | | | | | |
| M252Y/T256P/ | 168 | 9.27E+05 | 3.83E−05 | 4.14E−11 | 4.42E+05 | 2.00E−02 | 4.98E−08 |
| V308P/N434W | | | | | | | |
| M252Y/T256Y/ | 166 | 1.49E+06 | 1.12E−03 | 7.54E−10 | 3.95E+05 | 8.00E−02 | 2.02E−07 |
| V308P/N434W | | | | | | | |
| M252Y/T256V/ | 164 | 1.11E+06 | 1.39E−03 | 1.25E−09 | 9.77E+05 | 2.00E−01 | 2.02E−07 |
| V308P/N434F | | | | | | | |
| M252Y/T256H/ | 167 | 1.04E+06 | 2.28E−03 | 2.20E−09 | 1.82E+06 | 1.90E−01 | 1.03E−07 |
| V308P/N434W | | | | | | | |
| M252Y/T256A/ | 178 | 1.91E+06 | 8.15E−04 | 4.26E−10 | 8.93E+05 | 8.00E−02 | 9.01E−08 |
| V308P/N434W | | | | | | | |
| M252Y/T256M/ | 174 | 1.51E+06 | 1.25E−03 | 8.28E−10 | 7.62E+06 | 2.80E−01 | 3.72E−08 |
| V308P/N434W | | | | | | | |
| M252Y/T256K/ | 171 | 1.09E+06 | 1.43E−03 | 1.31E−09 | 4.57E+05 | 1.10E−01 | 2.39E−07 |
| V308P/N434W | | | | | | | |
| WT | 47 | 1.53E+06 | 1.03E+00 | 6.73E−07 | 6.25E+04 | 1.12E−03 | 1.80E−08 |

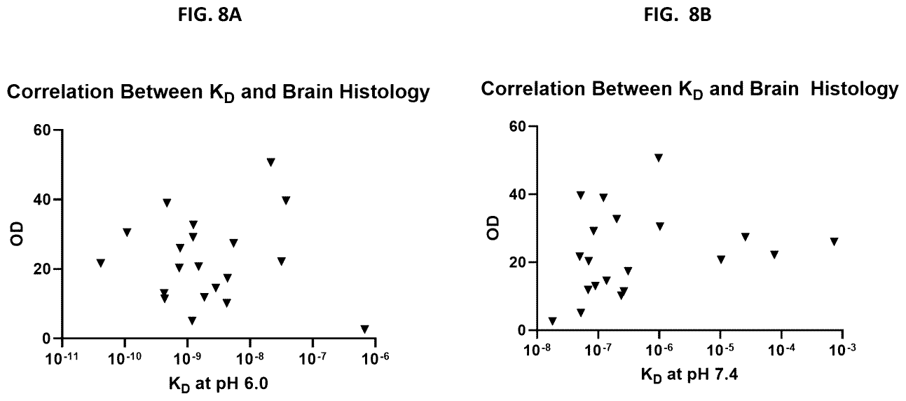

The antibody-hFcRn dissociation constant of each variant was plotted against the results from earlier brain immunohistochemistry assays as quantified in Examples 2, 3, and 5. Variants that were not tested on the brain immunohistochemistry assay was excluded from this analysis. As shown in FIG. 8, the antibody-FcRn dissociation constant has limited value in predicting which antibody variants would most efficiently distribute into the brain parenchyma. For example, the three antibody variants with the highest integrated OD values observed on Tables 5, 6, and 8 were M252Y/S254T/T256E/V308P, M252Y/V308P, and M252Y/S254T/T256E/V308P/N434W. The interaction between these three variants and FcRn had KD values that span over two orders of magnitude at pH 6.0, from 4.71×10⁻¹⁰ M to 2.15×10⁻⁸ M. Other variants that interacted with FcRn with similar affinity, such as M252Y/T256D/V308P/N434W (K_{D}=1.19×10⁻⁹), were detected in the brain parenchyma at OD levels not that different from wildtype antibody. Similarly, these three antibodies interacted with FcRn with a KD between 5.18×10⁻⁸ and 9.75×10⁻⁷ at pH 7.4. This affinity is similar to the affinity of WT antibody for FcRn at pH 7.4. This poor correlation stands in contrast to the Antibody Internalization Assay described in Example 4 and FIG. 3. In contrast, when antibodies were highly internalized in the Antibody Internalization Assay described in Example 4, they were also likely to be well-distributed into the brain parenchyma when administered to Tg276 mice.

### Example 11: The Effects of Additional Substitutions on the M252Y/V308P and M252Y/S254T/T256E/V308P/N434W Variant Backgrounds on Transport in Mouse Brains Expressing a Human FcRn Transgene after In Vivo Administration

Certain modifications in the constant region or Fc region of antibody proteins that increase the quantity of the molecule in the brain parenchyma after in vivo administration can be combined with other modifications that alter the serum-half life, the effector function, the glycosylation, or the physiochemical stability or homogeneity of the antibody protein.

For example, antibodies comprising either the M252Y/V308P or M252Y/S254T/T256E/V308P/N434W substitutions can be combined with modifications selected from Table 12, depending on the isotype of the antibody. A Δ in Table 12 indicates the amino acid is deleted.

TABLE 12
Integrated OD of M252Y/S254T/T256E/V308P/N434W
and M252Y/V308P antibody variants in Tg276 mice

| | | Additional | Base C_{H} |
|---|---|---|---|
| Isotype | Variant | Substitution(s) | SEQ ID NO: |
| Human IgG1 | M252Y/S254T/T256E/ | L235E | 703 |
| | V308P/N434W | | |
| Human IgG1 | M252Y/S254T/T256E/ | L235A/G237A | 704 |
| | V308P/N434W | | |
| Human IgG1 | M252Y/S254T/T256E/ | L235E/P329G | 705 |
| | V308P/N434W | | |
| Human IgG1 | M252Y/S254T/T256E/ | N297A | 706 |
| | V308P/N434W | | |
| Human IgG1 | M252Y/S254T/T256E/ | A330S/P331S | 707 |
| | V308P/N434W | | |
| Human IgG2 | M252Y/S254T/T256E/ | V235E | 708 |
| | V308P/N434W | | |
| Human IgG2 | M252Y/S254T/T256E/ | V235A/G237A | 709 |
| | V308P/N434W | | |
| Human IgG2 | M252Y/S254T/T256E/ | V235E/P329G | 710 |
| | V308P/N434W | | |
| Human IgG2 | M252Y/S254T/T256E/ | N297A | 711 |
| | V308P/N434W | | |
| Human IgG2 | M252Y/S254T/T256E/ | A330S/P331S | 712 |
| | V308P/N434W | | |
| Human IgG4 | M252Y/S254T/T256E/ | L235E | 713 |
| | V308P/N434W | | |
| Human IgG4 | M252Y/S254T/T256E/ | L235A/G237A | 714 |
| | V308P/N434W | | |
| Human IgG4 | M252Y/S254T/T256E/ | N297A | 715 |
| | V308P/N434W | | |
| Human IgG4 | M252Y/S254T/T256E/ | L235E/P329G | 716 |
| | V308P/N434W | | |
| Human IgG1 | M252Y/V308P | L235E | 717 |
| Human IgG1 | M252Y/V308P | L235A/G237A | 718 |
| Human IgG1 | M252Y/V308P | L235E/P329G | 719 |
| Human IgG1 | M252Y/V308P | N297A | 720 |
| Human IgG1 | M252Y/V308P | A330S/P331S | 721 |
| Human IgG2 | M252Y/V308P | V235E | 722 |
| Human IgG2 | M252Y/V308P | V235A/G237A | 723 |
| Human IgG2 | M252Y/V308P | V235E/P329G | 724 |
| Human IgG2 | M252Y/V308P | N297A | 725 |
| Human IgG2 | M252Y/V308P | A330S/P331S | 726 |
| Human IgG4 | M252Y/V308P | L235E | 727 |
| Human IgG4 | M252Y/V308P | L235A/G237A | 728 |
| Human IgG4 | M252Y/V308P | N297A | 729 |
| Human IgG4 | M252Y/V308P | L235E/P329G | 730 |

The antibody variants are tested as described in Example 4 to verify that they are still internalized by JEG3 human endothelial cells in a similar manner as their parents, M252Y/V308P and M252Y/S254T/T256E/V308P/N434W. In addition, to verify the quantity of these variants in the brain parenchyma after parenteral administration, the methods of Example 5 are used to measure the efficiency with which these variants distribute into the brain parenchyma.

### Example 12: Testing Delivery of Fluorescently-Tagged Small Molecules to the Brain

It may be advantageous to use antibody proteins to deliver small molecule drugs into the brain. These antibody-drug conjugates may comprise an antibody that binds cells expressing certain receptors and a drug that may exert a desired therapeutic effect. To test whether the anti-O4 antibody variant comprising the M252Y/S254T/T256E/V308P/N434W substitution used in Example 2 can shuttle small molecule drugs into the brain parenchyma, chemical moieties comprising Alexa Fluor 594 (ThermoFisher A20185) were conjugated to the antibody following the manufacturer's instructions. The amount of small molecule found in the brain was then quantified by fluorescence microscopy.

In brief, conjugated test antibodies were administered intravenously at 30 mg/kg to adult male Tg276 mice. Following inhalation anesthesia at 48 hours post-dosing, the animals were transcardially perfused with 50 mL of saline at a rate of 10 mL/min for 5 min or longer until the exiting fluid was clear. The saline perfusion step removed test materials from the luminal compartment of brain blood vessels, including the capillaries, and allowed quantification of the amount of test material found in the brain parenchyma. Brain samples were dissected out from each animal and placed into individual uniquely labeled pre-weighed 20 ml disposable scintillation vials. Vials were flash frozen on liquid nitrogen then placed on dry ice until stored at −80° C.

Brains were freeze-sectioned at 18 μm thickness in the coronal plane. Sections were allowed to air-dry at room temperature for 10-15 min and then fixed in room temperature 4% PFA for 15 min. Fixed sections were then rinsed in phosphate-buffered saline (pH 7.2) and counterstained with DAPI to mark cell nuclei. Stained sections were then coverslipped to visualize direct fluorescence from the conjugated Alexa594 fluorophore.

Slides with brain sections were imaged at 20× (conditions for Alexa594 detection: λex 590, λem 618, exposure 210 msec). Analysis of the resulting CZI images was performed on HALO (v3.2, Indica Labs, Albuquerque, NM, USA). For the image analysis, a minimum of 2 rostral to caudal coronal brain sections were selected from each animal. To ensure approximately the same brain regions were compared between different animals, images of sections were also matched based on gross anatomical landmarks. Images of sections from 1 animal were analyzed for each test antibody. Using HALO, an annotation contour was drawn along the margins of each coronal brain section in order to include all brain regions in the immunofluorescence staining analysis of the section. The HALO tissue classifier module was applied to the images, which allowed separation of the tissue in the image from non-tissue classes (i.e., glass) based on artificial intelligence random forest algorithm (Breiman L. (2001) Random Forests. Mach Learn. 45: 5-32), trained during a learning session on several representative images. The resulting Tissue-Glass Classifier parameters were then applied to all subsequent images analyzed. The Area Quantification FL module of HALO, designed specifically for immunofluorescent staining analysis, was used to quantify the intensity of the Alexa594 fluorophore staining within the tissue portion of the selected brain section images. Similar to the Tissue-Glass Classifier, parameters for the Area Quantification FL module were established over a training session on a few representative images in order to teach the algorithm to identify the Alexa594 staining within the DAPI counterstained brain section image. During the training session, parameters were established categorizing the Alexa594-only immunofluorescence staining signal as ‘weak,’ ‘moderate,’ or ‘strong’ based on pixel density. The resulting customized Area Quantification FL analysis module was then applied to all subsequent images analyzed. Following HALO analysis completion, the ‘Integrated Alexa Fluor 594 Average Positive Intensity’ for that section image was calculated as the product of the average Alexa594 positive intensity across all pixels in the annotation contour multiplied by the percentage of the Alexa594 positive area and recorded in Table 13.

TABLE 13
Average Integrated Alexa Fluor 594 Immunofluorescence Intensity

| | C_{H} SEQ | Average Integrated Alexa Fluor |
|---|---|---|
| Antibody Variant | ID NO: | 594 Average Positive Intensity |
| WT | 47 | 1,224.6 |
| M252Y/S254T/T256E/ | 36 | 33,603.9 |
| V308P/N434W | | |

As shown in Table 13, systemically administered anti-O4 antibodies comprising the M252Y/S254T/T256E/V308P/N434W substitution in their Fc domains were able to deliver more fluorescent small molecules to the brain parenchyma than anti-O4 antibodies with wild type Fc domains.

### Example 13: Testing Delivery of Lysosomal Enzymes to the Brain

It may be advantageous to use antibody proteins to deliver large biologic molecules into the brain. These drugs can comprise an active enzyme fused to the Fc region of an antibody protein. The active enzyme may be beta-glucuronidase, which can be used to treat Sly syndrome. The Fc region may comprise either the M252Y/V308P or the M252Y/S254T/T256E/V308P/N434W substitution. To test the delivery of the drug in a mouse model of these diseases, mouse stock 005643 may be bred at Jackson Laboratories. Littermates may be administered either the beta-glucuronidase Fc fusion or just the Fc region of the antibody protein. Development of the mice may be monitored for growth retardation, shortened extremities, and facial dysmorphism. At weaning age, the animals may be euthanized, and the brains dissected into hemispheres. One hemisphere can be processed according to Example 5 to detect drug article in the brain by immunohistochemistry. The other hemisphere may be processed to measure the accumulation of glycosaminoglycans in the brain.

Alternatively, the active enzyme may be N-acetylglucosaminidase, which can be used to treat Sanfilippo Syndrome. The Fc region may again comprise either the M252Y/V308P or the M252Y/S254T/T256E/V308P/N434W substitution. To test the delivery of the drug, mouse stock 005643 may be bred at Jackson Laboratories. Littermates may be administered either the N-acetylglucosaminidase Fc fusion or just the Fc region of the antibody protein. At 8 months of age, the animals may be euthanized, and the brains dissected into hemispheres. One hemisphere can be processed according to Example 5 to detect drug article in the brain by immunohistochemistry. The other hemisphere may be processed to measure the accumulation of heparin sulfate in the brain.

### Example 14: Quantifying Antibodies in the Brain of a Non-Human Primate

To test whether the antibody Fc variants described in earlier examples can be used to deliver antibody compounds to the brain of other animal model systems, we adapted the methods used in Example 1 to evaluate the brain penetrance of antibody variants in a primate in vivo model using immunohistochemistry.

Male cynomolgus monkeys between 1.5 to 6 kg were allowed to acclimate to the testing facility for 10 days prior to dosing with antibody. Animal body weights were recorded prior to dose administration. Test antibodies comprising a CH region indicated in Table 14 and targeting a primate cell-surface antigen in the CNS were administered intravenously at 30 mg/kg through a 60-minute saline infusion. At 72 hours post-dosing, animals received 0.01 mg/kg buprenorphine followed by 10-15 mg/kg ketamine intramuscularly. Afterwards, animals were administered sodium pentobarbital (20-50 mg/kg intravenously as needed) followed by exsanguination by whole-body perfusion with phosphate-buffered saline and 4% paraformaldehyde. Brain samples were dissected out from each animal after the animal had been fully perfused. Whole brains were then post-fixed in a 4% w/v PFA solution for 24 hours, after which they were transferred into phosphate buffered saline (PBS) for storage. The brains were then processed according to Example 1 section D for immunohistochemical analysis. The observations from this analysis are recorded in Table 14.

TABLE 14
Integrated OD of hIgG1 antibody variants in cynomolgus monkeys

| | C_{H} SEQ | Integrated |
|---|---|---|
| Antibody Fc Variant | ID NO: | OD |
| WT | 47 | 17.0 |
| M252Y/S254T/T256E/V308P/N434W | 36 | 90.5 |
| M252Y/V308P | 52 | 19.3 |
| M252Y/S254T/T256E/V308P/N434Y | 6 | 109.4 |
| M252Y/S254T/T256E/V308P/N434F | 405 | 66.5 |
| M252Y/S254T/T256E/V308P/N434H | 603 | 113.4 |
| M252Y/S254T/V308P/N434W | 55 | 40.2 |
| M252Y/T256E/V308P/N434W | 41 | 127.7 |

As shown in Table 14, many antibodies comprising Fc variants which were demonstrated in earlier examples to confer superior brain penetrance capabilities in Tg276 mice when compared with wild-type Fc containing antibodies also efficiently entered the brain of non-human primates.

### Example 15: Further Sequence Modifications

A series of Fc variants were designed in an attempt to improve the properties, such as potential immunogenicity, of an Fc variant bearing the substitutions M252Y/S254T/T256E/V308P/N434W, described here as YTEPW. Variants with additional substitutions to YTEPW were designed on an IgG1 and IgG4 backbones and are set out Table 15. The sequences in Table 15 are annotated to identify the various domains and substitutions, with CH1 domain double underlined, the Hinge in bold text, CH2 domain single underlined, CH3 domain italicized and additional substitutions identified in larger font.

In some cases, the IgG4 constant region was also substituted with the S228P substitution in the hinge region which is known to stabilize heavy chain pairing in this antibody isotype.

TABLE 15
Additional Fc variants based on YTEPW

| Substitu- | | | |
|---|---|---|---|
| tions | | Constant region | |
| (according | | sequence with | |
| to EU | Abbreviated | YTEPW on IgG1 | Constant Region sequence with |
| numbering) | description | backbone | YTEPW on IgG4 backbone |
| M252Y, | YTEP | ASTKGPSVFPLAPSSKSTSG | ASTKGPSVFPLAPCSRSTSESTAALGC |
| S254T, | W | GTAALGCLVKDYFPEPVTVS | LVKDYFPEPVTVSWNSGALTSGVHTFP |
| T256E, | | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| N434W | | YICNVNHKPSNTKVDKKVEP | CPAPEFLGGPSVFLFPPKPKDTLYITR |
| | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 732) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 731) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302E | WNSGALTSGVHTFPAVLQSS | AVLQSSGLYSLSSVVTVPSSSLGTKTY |
| V302E, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 734) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 733) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309G | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309G, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 736) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 735) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303Q | WNSGALTSGVHTFPAVLQSS | AVLQSSGLYSLSSVVTVPSSSLGTKTY |
| V303Q, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRV [symbol] SVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRV [symbol] SVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 738) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 737) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303G | WNSGALTSGVHTFPAVLQSS | AVLQSSGLYSLSSVVTVPSSSLGTKTY |
| V303G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRV [symbol] SVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRV [symbol] SVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 740) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 739) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309E | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309E, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 742) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 741) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | R301D | WNSGALTSGVHTFPAVLQSS | AVLQSSGLYSLSSVVTVPSSSLGTKTY |
| R301D, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTY [symbol] VVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STY [symbol] VVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 744) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 743) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | S304E | WNSGALTSGVHTFPAVLQSS | AVLQSSGLYSLSSVVTVPSSSLGTKTY |
| S304E, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYRVV [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 746) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 745) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | R301E | WNSGALTSGVHTFPAVLQSS | AVLQSSGLYSLSSVVTVPSSSLGTKTY |
| R301E, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTY [symbol] VVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STY [symbol] VVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 748) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 747) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | R301G | WNSGALTSGVHTFPAVLQSS | AVLQSSGLYSLSSVVTVPSSSLGTKTY |
| R301G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTY [symbol] VVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STY [symbol] VVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 750) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 749) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303H | WNSGALTSGVHTFPAVLQSS | AVLQSSGLYSLSSVVTVPSSSLGTKTY |
| V303H, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRV [symbol] SVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRV [symbol] SVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 752) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 751) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306Y | WNSGALTSGVHTFPAVLQSS | AVLQSSGLYSLSSVVTVPSSSLGTKTY |
| L306Y, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 754) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 753) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | S304D | WNSGALTSGVHTFPAVLQSS | AVLQSSGLYSLSSVVTVPSSSLGTKTY |
| S304D, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVV [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 756) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 755) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302G | WNSGALTSGVHTFPAVLQSS | AVLQSSGLYSLSSVVTVPSSSLGTKTY |
| V302G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 758) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 757) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V305G | WNSGALTSGVHTFPAVLQSS | AVLQSSGLYSLSSVVTVPSSSLGTKTY |
| V305G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVS [symbol] LTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVS [symbol] LTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 760) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 759) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | T307E | WNSGALTSGVHTFPAVLQSS | AVLQSSGLYSLSSVVTVPSSSLGTKTY |
| T307E, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYRVVSVL [symbol] PL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVL [symbol] PLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 762) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 761) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | T307D | WNSGALTSGVHTFPAVLQSS | AVLQSSGLYSLSSVVTVPSSSLGTKTY |
| T307D, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVL [symbol] PL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVL [symbol] PLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 764) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 763) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306P | WNSGALTSGVHTFPAVLQSS | AVLQSSGLYSLSSVVTVPSSSLGTKTY |
| L306P, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 766) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 765) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306K | WNSGALTSGVHTFPAVLQSS | AVLQSSGLYSLSSVVTVPSSSLGTKTY |
| L306K, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 768) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 767) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V305D | WNSGALTSGVHTFPAVLQSS | AVLQSSGLYSLSSVVTVPSSSLGTKTY |
| V305D, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVS [symbol] LTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVS [symbol] LTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 770) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 769) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306G | WNSGALTSGVHTFPAVLQSS | AVLQSSGLYSLSSVVTVPSSSLGTKTY |
| L306G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 772) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 771) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306R | WNSGALTSGVHTFPAVLQSS | AVLQSSGLYSLSSVVTVPSSSLGTKTY |
| L306R, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 774) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 773) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V305E | WNSGALTSGVHTFPAVLQSS | AVLQSSGLYSLSSVVTVPSSSLGTKTY |
| V305E, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVS [symbol] LTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVS [symbol] LTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 776) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 775) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306T | WNSGALTSGVHTFPAVLQSS | AVLQSSGLYSLSSVVTVPSSSLGTKTY |
| L306T, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 778) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 777) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306E | WNSGALTSGVHTFPAVLQSS | AVLQSSGLYSLSSVVTVPSSSLGTKTY |
| L306E, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 780) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 779) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306H | WNSGALTSGVHTFPAVLQSS | AVLQSSGLYSLSSVVTVPSSSLGTKTY |
| L306H, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 782) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 781) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306Q | WNSGALTSGVHTFPAVLQSS | AVLQSSGLYSLSSVVTVPSSSLGTKTY |
| L306Q, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 784) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 783) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309K | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309K, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 786) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 785) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303E | WNSGALTSGVHTFPAVLQSS | AVLQSSGLYSLSSVVTVPSSSLGTKTY |
| V303E, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRV [symbol] SVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRV [symbol] SVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 788) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 787) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306V | WNSGALTSGVHTFPAVLQSS | AVLQSSGLYSLSSVVTVPSSSLGTKTY |
| L306V, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | |
| | | LTKNQVSLTCLVKGFYPSDI | QEGNVFSCSVMHEALHWHYTQKSLSLS |
| | | AVEWESNGQPENNYKTTPPV | LG (SEQ ID NO: 790) |
| | | LDSDGSFFLYSKLTVDKSRW | |
| | | QQGNVFSCSVMHEALHWHYT | |
| | | QKSLSLSPG (SEQ ID NO: | |
| | | 789) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303K | WNSGALTSGVHTFPAVLQSS | AVLQSSGLYSLSSVVTVPSSSLGTKTY |
| V303K, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRV [symbol] SVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRV [symbol] SVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 792) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 791) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309Q | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| L309Q, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 794) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 793) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309T | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309T, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVL [symbol] PTHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 796) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 795) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | S304G | WNSGALTSGVHTFPAVLQSS | AVLQSSGLYSLSSVVTVPSSSLGTKTY |
| S304G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVV [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 798) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 797) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309S | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309S, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPSHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 800) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 799) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300P | WNSGALTSGVHTFPAVLQSS | AVLQSSGLYSLSSVVTVPSSSLGTKTY |
| Y300P, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQENST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 802) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 801) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300D | WNSGALTSGVHTFPAVLQSS | AVLQSSGLYSLSSVVTVPSSSLGTKTY |
| Y300D, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 804) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 803) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300G | WNSGALTSGVHTFPAVLQSS | AVLQSSGLYSLSSVVTVPSSSLGTKTY |
| Y300G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | |
| | | LTKNQVSLTCLVKGFYPSDI | QEGNVFSCSVMHEALHWHYTQKSLSLS |
| | | AVEWESNGQPENNYKTTPPV | LG (SEQ ID NO: 806) |
| | | LDSDGSFFLYSKLTVDKSRW | |
| | | QQGNVFSCSVMHEALHWHYT | |
| | | QKSLSLSPG (SEQ ID NO: | |
| | | 805) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302K | WNSGALTSGVHTFPAVLQSS | AVLQSSGLYSLSSVVTVPSSSLGTKTY |
| V302K, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 808) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 807) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302T | WNSGALTSGVHTFPAVLQSS | AVLQSSGLYSLSSVVTVPSSSLGTKTY |
| V302T, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 810) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 809) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | S304P | WNSGALTSGVHTFPAVLQSS | AVLQSSGLYSLSSVVTVPSSSLGTKTY |
| S304P, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVV [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 812) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 811) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | T307G | WNSGALTSGVHTFPAVLQSS | AVLQSSGLYSLSSVVTVPSSSLGTKTY |
| T307G, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVL [symbol] PL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVL [symbol] PLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 814) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 813) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V305P | WNSGALTSGVHTFPAVLQSS | AVLQSSGLYSLSSVVTVPSSSLGTKTY |
| V305P, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVS [symbol] LTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVS [symbol] LTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 816) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 815) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300Q | WNSGALTSGVHTFPAVLQSS | AVLQSSGLYSLSSVVTVPSSSLGTKTY |
| Y300Q, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 818) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 817) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300T | WNSGALTSGVHTFPAVLQSS | AVLQSSGLYSLSSVVTVPSSSLGTKTY |
| Y300T, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 820) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 819) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | R301Q | WNSGALTSGVHTFPAVLQSS | AVLQSSGLYSLSSVVTVPSSSLGTKTY |
| R301Q, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTY [symbol] VVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STY [symbol] VVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | |
| | | LTKNQVSLTCLVKGFYPSDI | QEGNVFSCSVMHEALHWHYTQKSLSLS |
| | | AVEWESNGQPENNYKTTPPV | LG (SEQ ID NO: 822) |
| | | LDSDGSFFLYSKLTVDKSRW | |
| | | QQGNVFSCSVMHEALHWHYT | |
| | | QKSLSLSPG (SEQ ID NO: | |
| | | 821) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302Q | WNSGALTSGVHTFPAVLQSS | AVLQSSGLYSLSSVVTVPSSSLGTKTY |
| V302Q, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 824) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 823) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | S304N | WNSGALTSGVHTFPAVLQSS | AVLQSSGLYSLSSVVTVPSSSLGTKTY |
| S304N, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYRVV [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 826) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 825) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306S | WNSGALTSGVHTFPAVLQSS | AVLQSSGLYSLSSVVTVPSSSLGTKTY |
| L306S, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVV [symbol] VSTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] SVSTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 828) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 827) | |
| M252Y, | YTEW- | ASTKGPSVFPLAPSSKSTSG | ASTKGPSVFPLAPCSRSTSESTAALGC |
| S254T, | V308H | GTAALGCLVKDYFPEPVTVS | LVKDYFPEPVTVSWNSGALTSGVHTFP |
| T256E, | | WNSGALTSGVHTFPAVLQSS | AVLQSSGLYSLSSVVTVPSSSLGTKTY |
| V308H, | | GLYSLSSVVTVPSSSLGTQT | TCNVDHKPSNTKVDKRVESKYGPPCPP |
| N434W | | YICNVNHKPSNTKVDKKVEP | CPAPEFLGGPSVFLFPPKPKDTLYITR |
| | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLT [symbol] L |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLT [symbol] LHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 830) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 829) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309P | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 832) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 831) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309V | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309V, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 834) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 833) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303S | WNSGALTSGVHTFPAVLQSS | AVLQSSGLYSLSSVVTVPSSSLGTKTY |
| V303S, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVS [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVS [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 836) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 835) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V305Q | WNSGALTSGVHTFPAVLQSS | AVLQSSGLYSLSSVVTVPSSSLGTKTY |
| V305Q, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVS [symbol] LTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVS [symbol] LTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | |
| | | LTKNQVSLTCLVKGFYPSDI | QEGNVFSCSVMHEALHWHYTQKSLSLS |
| | | AVEWESNGQPENNYKTTPPV | LG (SEQ ID NO: 838) |
| | | LDSDGSFFLYSKLTVDKSRW | |
| | | QQGNVFSCSVMHEALHWHYT | |
| | | QKSLSLSPG (SEQ ID NO: | |
| | | 837) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309H | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309H, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTPHHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 840) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 839) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302H | WNSGALTSGVHTFPAVLQSS | AVLQSSGLYSLSSVVTVPSSSLGTKTY |
| V302H, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| | | KSCDKTHTCPPCPAPELLGG | EPEVTCVVVDVSQEDPEVQFNWYVDGV |
| V308P, | | PSVFLFPPKPKDTLYITREP | EVHNAKTKPREEQFNSTYR [symbol] VSVLTPL |
| N434W | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 842) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 841) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302S | WNSGALTSGVHTFPAVLQSS | AVLQSSGLYSLSSVVTVPSSSLGTKTY |
| V302S, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 844) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 843) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300S | WNSGALTSGVHTFPAVLQSS | AVLQSSGLYSLSSVVTVPSSSLGTKTY |
| Y300S, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 847) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 846) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309R | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309R, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | |
| | | LTKNQVSLTCLVKGFYPSDI | QEGNVFSCSVMHEALHWHYTQKSLSLS |
| | | AVEWESNGQPENNYKTTPPV | LG (SEQ ID NO: 849) |
| | | LDSDGSFFLYSKLTVDKSRW | |
| | | QQGNVFSCSVMHEALHWHYT | |
| | | QKSLSLSPG (SEQ ID NO: | |
| | | 848) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300N | WNSGALTSGVHTFPAVLQSS | AVLQSSGLYSLSSVVTVPSSSLGTKTY |
| Y300N, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 851) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 850) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302R | WNSGALTSGVHTFPAVLQSS | AVLQSSGLYSLSSVVTVPSSSLGTKTY |
| V302R, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 853) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 852) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V303R | WNSGALTSGVHTFPAVLQSS | AVLQSSGLYSLSSVVTVPSSSLGTKTY |
| V303R, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRV [symbol] SVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRV [symbol] SVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 855) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 854) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L306A | WNSGALTSGVHTFPAVLQSS | AVLQSSGLYSLSSVVTVPSSSLGTKTY |
| L306A, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSV [symbol] TPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSV [symbol] TPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 857) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 856) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | L309A | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| L309A, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTP [symbol] |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTP [symbol] HQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 859) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 858) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | H310G | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| H310G, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTPL |
| | | EVTCVVVDVSHEDPEVKENW | [symbol] QDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTPL [symbol] QDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 861) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 860) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300H | WNSGALTSGVHTFPAVLQSS | AVLQSSGLYSLSSVVTVPSSSLGTKTY |
| Y300H, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 863) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 862) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | S304T | WNSGALTSGVHTFPAVLQSS | AVLQSSGLYSLSSVVTVPSSSLGTKTY |
| S304T, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVV [symbol] VLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVV [symbol] VLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | |
| | | LTKNQVSLTCLVKGFYPSDI | QEGNVFSCSVMHEALHWHYTQKSLSLS |
| | | AVEWESNGQPENNYKTTPPV | LG (SEQ ID NO: 865) |
| | | LDSDGSFFLYSKLTVDKSRW | |
| | | QQGNVFSCSVMHEALHWHYT | |
| | | QKSLSLSPG (SEQ ID NO: | |
| | | 864) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | H310D | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| H310D, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTPL |
| | | EVTCVVVDVSHEDPEVKENW | [symbol] QDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTPL [symbol] QDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 867) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 866) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | Y300K | WNSGALTSGVHTFPAVLQSS | AVLQSSGLYSLSSVVTVPSSSLGTKTY |
| Y300K, | | GLYSLSSVVTVPSSSLGTQT | TCNVDHKPSNTKVDKRVESKYGPPCPP |
| | | YICNVNHKPSNTKVDKKVEP | CPAPEFLGGPSVFLFPPKPKDTLYITR |
| V308P, | | KSCDKTHTCPPCPAPELLGG | EPEVTCVVVDVSQEDPEVQFNWYVDGV |
| N434W | | PSVFLFPPKPKDTLYITREP | EVHNAKTKPREEQFNST [symbol] RVVSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | ST [symbol] RVVSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 869) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 868) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | T307H | WNSGALTSGVHTFPAVLQSS | AVLQSSGLYSLSSVVTVPSSSLGTKTY |
| T307H, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVL [symbol] PL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVSVL [symbol] PLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 871) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 870) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | H310N | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| H310N, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQENSTYRVVSVLTPL |
| | | EVTCVVVDVSHEDPEVKENW | [symbol] QDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTPL [symbol] QDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 873) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 872) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V302A | WNSGALTSGVHTFPAVLQSS | AVLQSSGLYSLSSVVTVPSSSLGTKTY |
| V302A, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYR [symbol] VSVLTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYR [symbol] VSVLTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 875) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 874) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | V305K | WNSGALTSGVHTFPAVLQSS | AVLQSSGLYSLSSVVTVPSSSLGTKTY |
| V305K, | | GLYSLSSVVTVPSSSLGTQT | TCNVDHKPSNTKVDKRVESKYGPPCPP |
| V308P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVS [symbol] LTPL |
| | | EVTCVVVDVSHEDPEVKENW | HQDWLNGKEYKCKVSNKGLPSSIEKTI |
| | | YVDGVEVHNAKTKPREEQYN | SKAKGQPREPQVYTLPPSQEEMTKNQV |
| | | STYRVVS [symbol] LTPLHQDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 877) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 876) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | H310P | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| H310P, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTPL |
| | | EVTCVVVDVSHEDPEVKENW | [symbol] QDWLNGKEYKCKVSNKGLPSSIEKTI |
| | | YVDGVEVHNAKTKPREEQYN | SKAKGQPREPQVYTLPPSQEEMTKNQV |
| | | STYRVVSVLTPL [symbol] QDWLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 879) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 878) | |
| M252Y, | YTEP | ASTKGPSVFPLAPSSKSTSG | ASTKGPSVFPLAPCSRSTSESTAALGC |
| S254T, | W- | GTAALGCLVKDYFPEPVTVS | LVKDYFPEPVTVSWNSGALTSGVHTFP |
| T256E, | D312G | WNSGALTSGVHTFPAVLQSS | AVLQSSGLYSLSSVVTVPSSSLGTKTY |
| V308P, | | GLYSLSSVVTVPSSSLGTQT | TCNVDHKPSNTKVDKRVESKYGPPCPP |
| D312G, | | YICNVNHKPSNTKVDKKVEP | CPAPEFLGGPSVFLFPPKPKDTLYITR |
| N434W | | KSCDKTHTCPPCPAPELLGG | EPEVTCVVVDVSQEDPEVQFNWYVDGV |
| | | PSVFLFPPKPKDTLYITREP | EVHNAKTKPREEQFNSTYRVVSVLTPL |
| | | EVTCVVVDVSHEDPEVKENW | HQ [symbol] WLNGKEYKCKVSNKGLPSSIEKTI |
| | | YVDGVEVHNAKTKPREEQYN | SKAKGQPREPQVYTLPPSQEEMTKNQV |
| | | STYRVVSVLTPLHQ [symbol] WLNGK | SLTCLVKGFYPSDIAVEWESNGQPENN |
| | | EYKCKVSNKALPAPIEKTIS | YKTTPPVLDSDGSFFLYSRLTVDKSRW |
| | | KAKGQPREPQVYTLPPSRDE | QEGNVFSCSVMHEALHWHYTQKSLSLS |
| | | LTKNQVSLTCLVKGFYPSDI | LG (SEQ ID NO: 881) |
| | | AVEWESNGQPENNYKTTPPV | |
| | | LDSDGSFFLYSKLTVDKSRW | |
| | | QQGNVFSCSVMHEALHWHYT | |
| | | QKSLSLSPG (SEQ ID NO: | |
| | | 880) | |

### Example 16: Alternative Surface Plasmon Resonance Method

The affinity of selected Fc variants described in Table 15 for binding with human and human-mouse chimeric neonatal Fc receptor (FcRn) was investigated by surface plasmon resonance (SPR). Human IgG1 and human IgG4 antibodies were included as controls in this analysis. The experiments were conducted at 25° C. in HBS-EP buffer (0.01 M HEPES pH 7.4, 0.3 M NaCl, 3 mM EDTA, 0.1% BSA, 0.005% v/v Surfactant P20, pH 7.4). The pH was adjusted to pH 6.0 using 5 M HCl for acidic pH binding measurements. Test antibodies were captured to an immobilized Affini Pure anti-human IgG F(ab′)2 fragment to an approximate surface density of 60-90 RU. The binding kinetics (ka and kd) or steady-state binding affinity was determined by globally fitting the response data to a 1:1 interaction model. For kinetic evaluation, the affinity (KD) was calculated based on the ratio of the observed kinetics (kd/ka). For steady-state affinity, the KD values were calculated from a concentration response curve plotted using the steady state RU values (Req). Undetectable binding or binding that was too weak to enable accurate analysis was defined as not binding (DNB). In support of a relationship between FcRn binding and transcytosis, variant YTEPW-V302P, which lost detectable binding to the FcRn, was no longer internalized into hFcRn-positive cells.

TABLE 16
KD results by SPR for selected variants

KD (nM)

| Name | pH 6.0 | pH 7.4 |
|---|---|---|
| YTEPW-V302E | 2.3 | 143 |
| YTEPW-L309G | 2 | 118 |
| YTEPW-V303Q | 1.1 | 81 |
| YTEPW-L309E | 1.7 | 109 |
| YTEPW-L306Y | 1.1 | 70 |
| YTEPW-S304D | 1.5 | 82 |
| YTEPW-V302G | 88 | 137 |
| YTEPW-V305G | 6.9 | 197 |

### Example 17: Internalization Assays

The passage of an Fc-comprising molecule across the blood brain barrier is thought to occur through FcRn mediated transcytosis. Internalization into brain microvascular endothelial cells is a necessary initial step in this process.

The Incucyte® SX5 (Sartorius) instrument is used to demonstrate internalization of test antibodies in the immortalized FcRn-expressing human cell line HEK 293. HEK293 cells grown at 37° in humidified conditions, are plated in a 96 well plate at a density of 15,000 viable cells/well and incubated at 37° C. for 6 hours. Test antibodies are incubated with Incucyte® Fabfluor ph Antibody Labelling Reagent at a 1:1 molar concentration ratio for 15 minutes at 37° C., protected from light. The Fabfluor reagent is pH sensitive, emitting at a different wavelength when at pH conditions found in the endosome, and thus can detect the internalization of an antibody. 50 μl of Fabfluor-conjugated antibodies (1 μg/mL) are added to cells. Erbitux® (human IgG1) is used as positive control in this analysis. Images are recorded every 15 minutes for up to 6 six hours using a 10× magnification objective. Total antibody Integrated Intensity and mean area under the curve are determined for each treatment after phase confluence masking is applied.

In an alternative approach, a confocal microscope (ImageXpress® HT.ai Molecular Devices) was used to demonstrate internalization of test antibodies in JEG3 cells stably transfected with human FcRn. For the assay, test antibodies labelled with Alexa Fluor™ 488 were incubated with cells for 1, 2 and 4 hours. Cells were then washed with 0.2 molar Acetic Acid to remove staining at the cell membrane while allowing for the detection of internalized antibodies. Cells were then fixed with 4% paraformaldehyde solution for 20 minutes, washed twice with PBS and stored at 4° C. before image acquisition. Image analysis was done by quantifying total fluorescence intensity over total cell area analyzed in each region of interest. Antibodies were tested with 4 technical replicates per biological replicate and 3 biological replicates. Quantitation of internalization was performed using a custom python script and normalized fluorescence of each antibody compared to a human anti-Keyhole Limpet Haemocyanin IgG antibody.

TABLE 17
Internalization by fluorescence detection in JEG3-
hFcRN cells of selected variants at two hours.

| | Average fold change Alexa Fluor ™ 488 |
|---|---|
| Name | fluorescence compared to WT human IgG |
| YTEPW-L309G | 34.13 |
| YTEPW-V303Q | 21.44 |
| YTEPW-L309E | 13.68 |
| YTEPW-L306Y | 66.53 |
| YTEPW-S304D | 13.25 |

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A molecule which exhibits enhanced transport into the central nervous system, the molecule comprising a modified IgG Fc region which comprises the transport-enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to EU numbering, wherein the transport-enhancing amino acid substitutions enhance the transport of the molecule into the brain relative to a molecule comprising a non-modified IgG Fc region.

2. (canceled)

3. The molecule of claim 1, wherein said modified IgG Fc region further comprises one additional transport-enhancing amino acid substitution selected from the group consisting of S254T, T256D, T256E, T256H, T256L, T256N, T256P, T256Q, T256W, N434A, N434F, N434G, N434H, N434M, N434P, N434Q, N434R, N434S, and N434W, with amino acid residue numbering according to EU numbering.

4. (canceled)

5. The molecule of claim 1, wherein said modified IgG Fc region further comprises an additional two transport-enhancing amino substitutions selected from the group consisting of S254A/N434Y, S254F/N434Y, S254G/N434Y, S254H/N434Y, S254T/T256E, S254T/N434W, S254T/N434Y, S254T/N434F, S254T/N434H, T256A/N434F, T256A/N434S, T256A/N434W, T256A/N434Y, T256D/N434A, T256D/N434E, T256D/N434P, T256D/N434S, T256D/N434T, T256D/N434W, T256D/N434Y, T256E/N434A, T256E/N434F, T256E/N434G, T256E/N434H, T256E/N434P, T256E/N434Q, T256E/N434R, T256E/N434S, T256E/N434W, T256E/N434Y, T256F/N434F, T256F/N434R, T256F/N434S, T256F/N434W, T256F/N434Y, T256G/N434F, T256G/N434H, T256G/N434K, T256G/N434M, T256G/N434P, T256G/N434Q, T256G/N434R, T256G/N434S, T256G/N434W, T256G/N434Y, T256H/N434F, T256H/N434P, T256H/N434S, T256H/N434W, T256H/N434Y, T256I/N434I, T256I/N434T, T256I/N434V, T256I/N434W, T256I/N434Y, T256K/N434G, T256K/N434S, T256K/N434W, T256K/N434Y, T256L/N434F, T256L/N434I, T256L/N434K, T256L/N434W, T256L/N434Y, T256M/N434W, T256N/N434K, T256N/N434Y, T256P/N434A, T256P/N434F, T256P/N434G, T256P/N434H, T256P/N434I, T256P/N434K, T256P/N434M, T256P/N434W, T256P/N434Y, T256Q/N434L, T256Q/N434W, T256Q/N434Y, T256R/N434A, T256R/N434G, T256R/N434I, T256R/N434Q, T256R/N434S, T256R/N434V, T256R/N434W, T256R/N434Y, T256S/N434A, T256S/N434F, T256S/N434G, T256S/N434H, T256S/N434K, T256S/N434S, T256S/N434T, T256S/N434W, T256S/N434Y, T256V/N434F, T256V/N434G, T256V/N434I, T256V/N434M, T256V/N434R, T256V/N434T, T256V/N434W, T256V/N434Y, T256W/N434S, T256W/N434V, T256W/N434W, T256W/N434Y, T256Y/N434H, T256Y/N434S, T256Y/N434V, T256Y/N434W, and T256Y/N434Y, with amino acid residue numbering according to EU numbering.

6. (canceled)

7. The molecule of claim 5, wherein the additional two transport-enhancing amino acid substitutions are selected from the group selected from T256V/N434F; T256E/N434H; T256S/N434W; T256W/N434Y; T256E/N434F; T256R/N434Y; T256P/N434W; T256E/N434Y; T256F/N434F; T256W/N434W; T256F/N434Y; T256L/N434W; T256Q/N434W; T256E/N434W; T256A/N434W; T256E/N434P; T256V/N434W; T256I/N434Y; T256R/N434W; T256G/N434Y; T256L/N434Y; T256V/N434Y; T256Y/N434Y; T256N/N434Y; T256Q/N434Y; T256A/N434Y; T256P/N434Y; T256S/N434Y; T256D/N434W; and T256H/N434F, with amino acid residue numbering according to EU numbering.

8. The molecule of claim 1, wherein said modified IgG Fc region further comprises an additional three transport-enhancing amino substitutions selected from the group consisting of S254T/T256E/N434Y; S245T/T256E/N434F; and S254T/T256E/N434H, with amino acid residue numbering according to EU numbering.

9. The molecule of claim 1, wherein the transport-enhancing amino acid substitutions consist of M252Y, S254T, T256E, V308P, N434W.

10. The molecule of claim 1, wherein the modified IgG Fc is an IgG1 Fc.

11. The molecule of claim 10 further comprising one or more Fc modifications selected from the group consisting of L235A/G237A, N297A, A330S/P331S, L234A/L235A/P329G and K447Δ, with amino acid residue numbering according to EU numbering.

12. The molecule of claim 1, wherein the modified IgG Fc is an IgG2 Fc.

13. The molecule of claim 12, further comprising one or more Fc modifications selected from the group consisting of N297X, A330S/P331S, and K447Δ, with amino acid residue numbering according to EU numbering.

14. The molecule of claim 1, wherein the modified IgG Fc is an IgG4 Fc.

15. The molecule of claim 14, further comprising an additional alteration selected from S228P and/or one or more Fc modifications selected from the group consisting of N297A, P331S, and K447Δ, with amino acid residue numbering according to EU numbering.

16. The molecule of claim 1, wherein the amino acid sequence of the modified Fc region sequence comprises a sequence selected from SEQ ID NOs: 731-881.

17. The molecule of claim 1, wherein the molecule comprising the modified IgG Fc region comprises or consists of an antibody.

18. (canceled)

19. The molecule of claim 1, wherein the molecule binds to an extracellular antigen and/or a cell surface antigen in the CNS.

20. (canceled)

21. (canceled)

22. A molecule which exhibits enhanced transport into the brain, the molecule comprising a modified IgG Fc region, wherein said modified IgG Fc region comprises the transport enhancing amino acid substitution or substitutions selected from the group consisting of S254T/T256E/V308P/N434W; S254T/T256E/N434W; N286E/M428I/N434Y; N434W; T256E/N434W; T256E; S254T/T256E; and S254T/N434W and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G.

23. A fusion protein which exhibits enhanced blood-brain barrier transport, the fusion protein comprising a modified IgG Fc region, wherein said modified IgG Fc region comprises the blood-brain barrier transport enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to EU numbering, wherein the amino acid substitutions enhance the blood brain barrier transport of the molecule into the brain.

24-33. (canceled)

34. A method of enhancing delivery of a molecule comprising an IgG Fc region to the brain, the method comprising:
modifying the amino acid sequence of the IgG Fc region to comprise the transport-enhancing amino acid substitutions M252Y/V308P and one additional amino acid substitution selected from the group consisting of Y300Q, Y300T, Y300S, Y300K, Y300N, Y300P, Y300D, Y300G, Y300H R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering, wherein the amino acid substitutions enhance blood brain barrier transport and
administering the molecule to the brain.

35. A method of enhancing delivery of a molecule comprising an IgG Fc region to the brain, the method comprising:
modifying the amino acid sequence of the IgG Fc region to introduce amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering, wherein the amino acid substitutions enhance blood brain barrier transport.

36-46. (canceled)

47. A method of treating a disorder which is associated with the central nervous system, the method comprising:
administering a molecule comprising a modified IgG Fc region wherein said modified IgG Fc region comprises the transport-enhancing amino acid substitutions M252Y/V308P and one additional amino acid substitution selected from the group consisting of Y300Q, Y300T, Y300S, Y300K, Y300N, Y300P, Y300D, Y300G, Y300H, R301D, R301E, R301G, R301Q, V302A, V302E, V302G, V302K, V302T, V302H, V302S, V302R, V302Q, V303Q, v303G, V303H, V303E, V303K, V303S, V303R, S304E, S304D, S304G, S304P, S304N, S304T, V305G, V305K, V305D, V305E, V305P, V305Q, L306Y, L306P, L306K, L306G, L306R, L306T, L306E, L306H, L306Q, L306V, L306S, L306A, T307E, T307D, T307G, T307H, V308H, L309G, L309E, L309K, L309Q, L309T, L309S, L309P, L309V, L309H, L309R, L309A, H310G, H310D, H310N, H310P, and D312G, with amino acid residue numbering according to the EU numbering, and wherein the amino acid substitutions enhance blood brain barrier transport.

48-59. (canceled)


SEQUENCE LISTING (881 sequences)

SEQ ID NO: 1-3 - skipped (3 entries)

SEQ ID NO: 4 - protein, 107 aa, synthetic construct [sequence omitted]

SEQ ID NO: 5-34 - skipped (30 entries)

SEQ ID NO: 35 - protein, 106 aa, synthetic construct
  REGION 1..106: note = Human lambda light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36-528 - skipped (493 entries)

SEQ ID NO: 529 - protein, 217 aa, synthetic construct
  REGION 1..217: note = human IgG1 Fc fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 530-564 - skipped (35 entries)

SEQ ID NO: 565 - protein, 330 aa, synthetic construct [sequence omitted]

SEQ ID NO: 566-656 - skipped (91 entries)

SEQ ID NO: 657 - protein, 216 aa, synthetic construct
  REGION 1..216: note = human IgG2 Fc fragment
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 658 - protein, 326 aa, synthetic construct
  REGION 1..326: note = Human IgG2 CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 659 - protein, 217 aa, synthetic construct
  REGION 1..217: note = human IgG4 Fc fragment
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 660 - protein, 327 aa, synthetic construct
  REGION 1..327: note = Human IgG4 CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 661 - protein, 327 aa, synthetic construct
  REGION 1..327: note = Human IgG4 S228P CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 662 - protein, 217 aa, synthetic construct
  REGION 1..217: note = Human IgG1 L235E Fc fragment
APELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 663 - protein, 216 aa, synthetic construct
  REGION 1..216: note = Human IgG2 V235E Fc fragment
APPEAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 664 - protein, 217 aa, synthetic construct
  REGION 1..217: note = Human IgG4 L235E Fc fragment
APEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 665 - protein, 217 aa, synthetic construct
  REGION 1..217: note = Human IgG1 L235A/G237A Fc fragment
APELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 217 aa, synthetic construct
  REGION 1..217: note = Human IgG4 L235A/G237A Fc fragment
APEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 667 - protein, 216 aa, synthetic construct
  REGION 1..216: note = Human IgG2 V235A/G237A Fc fragment
APPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 668 - protein, 217 aa, synthetic construct
  REGION 1..217: note = Human IgG1 N297A Fc fragment
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 669 - protein, 216 aa, synthetic construct
  REGION 1..216: note = Human IgG2 N297A Fc fragment
APPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 670 - protein, 217 aa, synthetic construct
  REGION 1..217: note = Human IgG4 N297A Fc fragment
APEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 671 - protein, 217 aa, synthetic construct
  REGION 1..217: note = Human IgG1 A330S/P331S Fc fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 672 - protein, 216 aa, synthetic construct
  REGION 1..216: note = Human IgG2 A330S/P331S Fc fragment
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 673 - protein, 217 aa, synthetic construct
  REGION 1..217: note = Human IgG1 L235E/P329G Fc fragment
APELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 674 - protein, 217 aa, synthetic construct
  REGION 1..217: note = Human IgG4 L235E/P329G Fc fragment
APEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 675 - protein, 216 aa, synthetic construct
  REGION 1..216: note = Human IgG2 V235E/P329G Fc fragment
APPEAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLGAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 676 - protein, 216 aa, synthetic construct
  REGION 1..216: note = Human IgG1 K447delta Fc fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 677 - protein, 215 aa, synthetic construct
  REGION 1..215: note = Human IgG2 K447delta Fc fragment
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 678 - protein, 216 aa, synthetic construct
  REGION 1..216: note = Human IgG4 K447delta Fc fragment
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 679 - protein, 330 aa, synthetic construct
  REGION 1..330: note = Human IgG1 L235E CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 680 - protein, 330 aa, synthetic construct
  REGION 1..330: note = Human IgG1 L235A/G237A CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 681 - protein, 330 aa, synthetic construct
  REGION 1..330: note = Human IgG1 L235E/P329G CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 682 - protein, 330 aa, synthetic construct
  REGION 1..330: note = Human IgG1 N297A CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 683 - protein, 330 aa, synthetic construct
  REGION 1..330: note = Human IgG1 A330S/P331S CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 684 - protein, 329 aa, synthetic construct
  REGION 1..329: note = Human IgG1 K447delta CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 685 - protein, 326 aa, synthetic construct
  REGION 1..326: note = Human IgG2 V235E CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPEAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 686 - protein, 326 aa, synthetic construct
  REGION 1..326: note = Human IgG2 V235A/G237A CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 687 - protein, 326 aa, synthetic construct
  REGION 1..326: note = Human IgG2 V235E/P329G CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPEAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLGAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 688 - protein, 326 aa, synthetic construct
  REGION 1..326: note = Human IgG2 N297A CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 689 - protein, 326 aa, synthetic construct
  REGION 1..326: note = Human IgG2 A330S/P331S CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 690 - protein, 325 aa, synthetic construct
  REGION 1..325: note = Human IgG2 K447delta CH1-CH3
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 691 - protein, 327 aa, synthetic construct
  REGION 1..327: note = Human IgG4 L235E CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 692 - protein, 327 aa, synthetic construct
  REGION 1..327: note = Human IgG4 L235A/G237A CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 693 - protein, 327 aa, synthetic construct
  REGION 1..327: note = Human IgG4 N297A CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 694 - protein, 327 aa, synthetic construct
  REGION 1..327: note = Human IgG4 L235E/P329G CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 695 - protein, 326 aa, synthetic construct
  REGION 1..326: note = Human IgG4 K447delta CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 696-730 - skipped (35 entries)

SEQ ID NO: 731 - protein, 329 aa, synthetic construct
  REGION 1..329: note = YTEPW on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 732 - protein, 326 aa, synthetic construct
  REGION 1..326: note = YTEPW on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 733 - protein, 329 aa, synthetic construct
  REGION 1..329: note = YTEPW-V302E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYREVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 734 - protein, 326 aa, synthetic construct
  REGION 1..326: note = YTEPW-V302E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYREVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 735 - protein, 329 aa, synthetic construct
  REGION 1..329: note = YTEPW-L309G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPGHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 736 - protein, 326 aa, synthetic construct
  REGION 1..326: note = YTEPW-L309G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPGHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 737 - protein, 329 aa, synthetic construct
  REGION 1..329: note = YTEPW-V303Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVQSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 738 - protein, 326 aa, synthetic construct
  REGION 1..326: note = YTEPW-V303Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVQSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 739 - protein, 329 aa, synthetic construct
  REGION 1..329: note = YTEPW-V303G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVGSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 740 - protein, 326 aa, synthetic construct
  REGION 1..326: note = YTEPW-V303G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVGSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 741 - protein, 329 aa, synthetic construct
  REGION 1..329: note = YTEPW-L309E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPEHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 742 - protein, 326 aa, synthetic construct
  REGION 1..326: note = YTEPW-L309E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPEHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 743 - protein, 329 aa, synthetic construct
  REGION 1..329: note = YTEPW-R301D on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYDVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 744 - protein, 326 aa, synthetic construct
  REGION 1..326: note = YTEPW-R301D on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYDVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 745 - protein, 329 aa, synthetic construct
  REGION 1..329: note = YTEPW-S304E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVEVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 746 - protein, 326 aa, synthetic construct
  REGION 1..326: note = YTEPW-S304E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVEVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 747 - protein, 329 aa, synthetic construct
  REGION 1..329: note = YTEPW-R301E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYEVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 748 - protein, 326 aa, synthetic construct
  REGION 1..326: note = YTEPW-R301E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYEVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 749 - protein, 329 aa, synthetic construct
  REGION 1..329: note = YTEPW-R301G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYGVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 750 - protein, 326 aa, synthetic construct
  REGION 1..326: note = YTEPW-R301G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYGVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 751 - protein, 329 aa, synthetic construct
  REGION 1..329: note = YTEPW-V303H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVHSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 752 - protein, 326 aa, synthetic construct
  REGION 1..326: note = YTEPW-V303H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVHSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 753 - protein, 329 aa, synthetic construct
  REGION 1..329: note = YTEPW-L306Y on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVYTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 754 - protein, 326 aa, synthetic construct
  REGION 1..326: note = YTEPW-L306Y on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVYTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 755 - protein, 329 aa, synthetic construct
  REGION 1..329: note = YTEPW-S304D on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVDVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 756 - protein, 326 aa, synthetic construct
  REGION 1..326: note = YTEPW-S304D on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVDVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 757 - protein, 329 aa, synthetic construct
  REGION 1..329: note = YTEPW-V302G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRGVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 758 - protein, 326 aa, synthetic construct
  REGION 1..326: note = YTEPW-V302G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRGVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 759 - protein, 329 aa, synthetic construct
  REGION 1..329: note = YTEPW-V305G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSGLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 760 - protein, 326 aa, synthetic construct
  REGION 1..326: note = YTEPW-V305G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSGLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 761 - protein, 329 aa, synthetic construct
  REGION 1..329: note = YTEPW-T307E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLEPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 762 - protein, 326 aa, synthetic construct
  REGION 1..326: note = YTEPW-T307E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLEPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 763 - protein, 329 aa, synthetic construct
  REGION 1..329: note = YTEPW-T307D on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLDPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 764 - protein, 326 aa, synthetic construct
  REGION 1..326: note = YTEPW-T307D on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLDPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 765 - protein, 329 aa, synthetic construct
  REGION 1..329: note = YTEPW-L306P on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVPTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 766 - protein, 326 aa, synthetic construct
  REGION 1..326: note = YTEPW-L306P on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVPTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 767 - protein, 329 aa, synthetic construct
  REGION 1..329: note = YTEPW-L306K on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVKTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 768 - protein, 326 aa, synthetic construct
  REGION 1..326: note = YTEPW-L306K on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVKTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 769 - protein, 329 aa, synthetic construct
  REGION 1..329: note = YTEPW-V305D on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSDLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 770 - protein, 326 aa, synthetic construct
  REGION 1..326: note = YTEPW-V305D on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSDLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 771 - protein, 329 aa, synthetic construct
  REGION 1..329: note = YTEPW-L306G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVGTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 772 - protein, 326 aa, synthetic construct
  REGION 1..326: note = YTEPW-L306G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVGTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 773 - protein, 329 aa, synthetic construct
  REGION 1..329: note = YTEPW-L306R on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVRTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 774 - protein, 326 aa, synthetic construct
  REGION 1..326: note = YTEPW-L306R on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVRTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 775 - protein, 329 aa, synthetic construct
  REGION 1..329: note = YTEPW-V305E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSELTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 776 - protein, 326 aa, synthetic construct
  REGION 1..326: note = YTEPW-V305E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSELTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 777 - protein, 329 aa, synthetic construct
  REGION 1..329: note = YTEPW-L306T on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVTTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 778 - protein, 326 aa, synthetic construct
  REGION 1..326: note = YTEPW-L306T on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVTTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 779 - protein, 329 aa, synthetic construct
  REGION 1..329: note = YTEPW-L306E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVETPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 780 - protein, 326 aa, synthetic construct
  REGION 1..326: note = YTEPW-L306E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVETPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 781 - protein, 329 aa, synthetic construct
  REGION 1..329: note = YTEPW-L306H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVHTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 782 - protein, 326 aa, synthetic construct
  REGION 1..326: note = YTEPW-L306H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVHTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 783 - protein, 329 aa, synthetic construct
  REGION 1..329: note = YTEPW-L306Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVQTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 784 - protein, 326 aa, synthetic construct
  REGION 1..326: note = YTEPW-L306Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVQTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 785 - protein, 329 aa, synthetic construct
  REGION 1..329: note = YTEPW-L309K on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPKHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 786 - protein, 326 aa, synthetic construct
  REGION 1..326: note = YTEPW-L309K on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPKHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 787 - protein, 329 aa, synthetic construct
  REGION 1..329: note = YTEPW-V303E on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVESVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 788 - protein, 326 aa, synthetic construct
  REGION 1..326: note = YTEPW-V303E on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVESVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 789 - protein, 329 aa, synthetic construct
  REGION 1..329: note = YTEPW-L306V on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVVTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 790 - protein, 326 aa, synthetic construct
  REGION 1..326: note = YTEPW-L306V on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVVTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 791 - protein, 329 aa, synthetic construct
  REGION 1..329: note = YTEPW-V303K on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVKSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 792 - protein, 326 aa, synthetic construct
  REGION 1..326: note = YTEPW-V303K on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVKSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 793 - protein, 329 aa, synthetic construct
  REGION 1..329: note = YTEPW-L309Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPQHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 794 - protein, 326 aa, synthetic construct
  REGION 1..326: note = YTEPW-L309Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPQHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 795 - protein, 329 aa, synthetic construct
  REGION 1..329: note = YTEPW-L309T on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPTHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 796 - protein, 326 aa, synthetic construct
  REGION 1..326: note = YTEPW-L309T on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPTHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 797 - protein, 329 aa, synthetic construct
  REGION 1..329: note = YTEPW-S304G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVGVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 798 - protein, 326 aa, synthetic construct
  REGION 1..326: note = YTEPW-S304G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVGVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 799 - protein, 329 aa, synthetic construct
  REGION 1..329: note = YTEPW-L309S on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPSHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 800 - protein, 326 aa, synthetic construct
  REGION 1..326: note = YTEPW-L309S on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPSHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 801 - protein, 329 aa, synthetic construct
  REGION 1..329: note = YTEPW-Y300P on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTPRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 802 - protein, 326 aa, synthetic construct
  REGION 1..326: note = YTEPW-Y300P on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTPRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 803 - protein, 329 aa, synthetic construct
  REGION 1..329: note = YTEPW-Y300D on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTDRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 804 - protein, 326 aa, synthetic construct
  REGION 1..326: note = YTEPW-Y300D on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTDRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 805 - protein, 329 aa, synthetic construct
  REGION 1..329: note = YTEPW-Y300G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTGRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 806 - protein, 326 aa, synthetic construct
  REGION 1..326: note = YTEPW-Y300G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTGRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 807 - protein, 329 aa, synthetic construct
  REGION 1..329: note = YTEPW-V302K on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRKVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 808 - protein, 326 aa, synthetic construct
  REGION 1..326: note = YTEPW-V302K on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRKVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 809 - protein, 329 aa, synthetic construct
  REGION 1..329: note = YTEPW-V302T on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRTVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 810 - protein, 326 aa, synthetic construct
  REGION 1..326: note = YTEPW-V302T on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRTVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 811 - protein, 329 aa, synthetic construct
  REGION 1..329: note = YTEPW-S304P on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVPVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 812 - protein, 326 aa, synthetic construct
  REGION 1..326: note = YTEPW-S304P on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVPVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 813 - protein, 329 aa, synthetic construct
  REGION 1..329: note = YTEPW-T307G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLGPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 814 - protein, 326 aa, synthetic construct
  REGION 1..326: note = YTEPW-T307G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLGPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 815 - protein, 329 aa, synthetic construct
  REGION 1..329: note = YTEPW-V305P on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSPLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 816 - protein, 326 aa, synthetic construct
  REGION 1..326: note = YTEPW-V305P on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSPLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 817 - protein, 329 aa, synthetic construct
  REGION 1..329: note = YTEPW-Y300Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTQRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 818 - protein, 326 aa, synthetic construct
  REGION 1..326: note = YTEPW-Y300Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTQRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 819 - protein, 329 aa, synthetic construct
  REGION 1..329: note = YTEPW-Y300T on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTTRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 820 - protein, 326 aa, synthetic construct
  REGION 1..326: note = YTEPW-Y300T on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTTRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 821 - protein, 329 aa, synthetic construct
  REGION 1..329: note = YTEPW-R301Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYQVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 822 - protein, 326 aa, synthetic construct
  REGION 1..326: note = YTEPW-R301Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYQVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 823 - protein, 329 aa, synthetic construct
  REGION 1..329: note = YTEPW-V302Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRQVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 824 - protein, 326 aa, synthetic construct
  REGION 1..326: note = YTEPW-V302Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRQVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 825 - protein, 329 aa, synthetic construct
  REGION 1..329: note = YTEPW-S304N on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVNVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 826 - protein, 326 aa, synthetic construct
  REGION 1..326: note = YTEPW-S304N on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVNVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 827 - protein, 329 aa, synthetic construct
  REGION 1..329: note = YTEPW-L306S on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVSTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 828 - protein, 326 aa, synthetic construct
  REGION 1..326: note = YTEPW-L306S on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVSTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 829 - protein, 329 aa, synthetic construct
  REGION 1..329: note = YTEW-V308H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTHLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 830 - protein, 326 aa, synthetic construct
  REGION 1..326: note = YTEW-V308H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTHLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 831 - protein, 329 aa, synthetic construct
  REGION 1..329: note = YTEPW-L309P on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPPHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 832 - protein, 326 aa, synthetic construct
  REGION 1..326: note = YTEPW-L309P on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPPHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 833 - protein, 329 aa, synthetic construct
  REGION 1..329: note = YTEPW-L309V on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPVHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 834 - protein, 326 aa, synthetic construct
  REGION 1..326: note = YTEPW-L309V on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 835 - protein, 329 aa, synthetic construct
  REGION 1..329: note = YTEPW-V303S on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVSSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 836 - protein, 326 aa, synthetic construct
  REGION 1..326: note = YTEPW-V303S on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVSSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 837 - protein, 329 aa, synthetic construct
  REGION 1..329: note = YTEPW-V305Q on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSQLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 838 - protein, 326 aa, synthetic construct
  REGION 1..326: note = YTEPW-V305Q on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSQLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 839 - protein, 329 aa, synthetic construct
  REGION 1..329: note = YTEPW-L309H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPHHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 840 - protein, 326 aa, synthetic construct
  REGION 1..326: note = YTEPW-L309H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPHHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 841 - protein, 329 aa, synthetic construct
  REGION 1..329: note = YTEPW-V302H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRHVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 842 - protein, 326 aa, synthetic construct
  REGION 1..326: note = YTEPW-V302H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRHVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 843 - protein, 329 aa, synthetic construct
  REGION 1..329: note = YTEPW-V302S on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRSVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 844 - protein, 326 aa, synthetic construct
  REGION 1..326: note = YTEPW-V302S on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRSVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 845 - skipped

SEQ ID NO: 846 - protein, 329 aa, synthetic construct
  REGION 1..329: note = YTEPW-Y300S on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTSRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 847 - protein, 326 aa, synthetic construct
  REGION 1..326: note = YTEPW-Y300S on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTSRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 848 - protein, 329 aa, synthetic construct
  REGION 1..329: note = YTEPW-L309R on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPRHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 849 - protein, 326 aa, synthetic construct
  REGION 1..326: note = YTEPW-L309R on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPRHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 850 - protein, 329 aa, synthetic construct
  REGION 1..329: note = YTEPW-Y300N on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTNRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 851 - protein, 326 aa, synthetic construct
  REGION 1..326: note = YTEPW-Y300N on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTNRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 852 - protein, 329 aa, synthetic construct
  REGION 1..329: note = YTEPW-V302R on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRRVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 853 - protein, 326 aa, synthetic construct
  REGION 1..326: note = YTEPW-V302R on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRRVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 854 - protein, 329 aa, synthetic construct
  REGION 1..329: note = YTEPW-V303R on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVRSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 855 - protein, 326 aa, synthetic construct
  REGION 1..326: note = YTEPW-V303R on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVRSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 856 - protein, 329 aa, synthetic construct
  REGION 1..329: note = YTEPW-L306A on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVATPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 857 - protein, 326 aa, synthetic construct
  REGION 1..326: note = YTEPW-L306A on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVATPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 858 - protein, 329 aa, synthetic construct
  REGION 1..329: note = YTEPW-L309A on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPAHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 859 - protein, 326 aa, synthetic construct
  REGION 1..326: note = YTEPW-L309A on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPAHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 860 - protein, 329 aa, synthetic construct
  REGION 1..329: note = YTEPW-H310G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPLGQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 861 - protein, 326 aa, synthetic construct
  REGION 1..326: note = YTEPW-H310G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPLGQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 862 - protein, 329 aa, synthetic construct
  REGION 1..329: note = YTEPW-Y300H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTHRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 863 - protein, 326 aa, synthetic construct
  REGION 1..326: note = YTEPW-Y300H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTHRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 864 - protein, 329 aa, synthetic construct
  REGION 1..329: note = YTEPW-S304T on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVTVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 865 - protein, 326 aa, synthetic construct
  REGION 1..326: note = YTEPW-S304T on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVTVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 866 - protein, 329 aa, synthetic construct
  REGION 1..329: note = YTEPW-H310D on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPLDQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 867 - protein, 326 aa, synthetic construct
  REGION 1..326: note = YTEPW-H310D on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPLDQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 868 - protein, 329 aa, synthetic construct
  REGION 1..329: note = YTEPW-Y300K on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTKRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 869 - protein, 326 aa, synthetic construct
  REGION 1..326: note = YTEPW-Y300K on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTKRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 870 - protein, 329 aa, synthetic construct
  REGION 1..329: note = YTEPW-T307H on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLHPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 871 - protein, 326 aa, synthetic construct
  REGION 1..326: note = YTEPW-T307H on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLHPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 872 - protein, 329 aa, synthetic construct
  REGION 1..329: note = YTEPW-H310N on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPLNQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 873 - protein, 326 aa, synthetic construct
  REGION 1..326: note = YTEPW-H310N on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPLNQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 874 - protein, 329 aa, synthetic construct
  REGION 1..329: note = YTEPW-V302A on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRAVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 875 - protein, 326 aa, synthetic construct
  REGION 1..326: note = YTEPW-V302A on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRAVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 876 - protein, 329 aa, synthetic construct
  REGION 1..329: note = YTEPW-V305K on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSKLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 877 - protein, 326 aa, synthetic construct
  REGION 1..326: note = YTEPW-V305K on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSKLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 878 - protein, 329 aa, synthetic construct
  REGION 1..329: note = YTEPW-H310P on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPLPQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 879 - protein, 326 aa, synthetic construct
  REGION 1..326: note = YTEPW-H310P on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPLPQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG

SEQ ID NO: 880 - protein, 329 aa, synthetic construct
  REGION 1..329: note = YTEPW-D312G on IgG1 backbone
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTPLHQGWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 881 - protein, 326 aa, synthetic construct
  REGION 1..326: note = YTEPW-D312G on IgG4 backbone
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTPLHQGWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHWHYTQKSLSLSLG